(19) United States
(12) Patent Application Publication
(10) Pub. No.: US 2024/0226306 A1
(43) Pub. Date: Jul. 11, 2024

(54) TREM-2/DAP-12 INHIBITORS FOR TREATING LUNG DISEASE AND INJURY AND COMBINATIONS THEREOF

(71) Applicant: SIGNABLK, INC., Shrewsbury, MA (US)

(72) Inventor: Alexander Sigalov, Worcester, MA (US)

(21) Appl. No.: 18/560,300

(22) PCT Filed: May 5, 2022
(86) PCT No.: PCT/US2022/027836

§ 371 Date: Nov. 10, 2023

Related U.S. Application Data
(60) Provisional application No. 63/190,497, filed on May 19, 2021.

(51) Int. Cl. A61K 47/54; A61P 11/00; A61P 29/00; C07K 7/04; A61K 38/00

(52) U.S. Cl. CPC A61K 47/554; A61P 11/00; A61P 29/00; C07K 7/04; A61K 38/00

(57) ABSTRACT

The present invention is related to the field of pulmonary therapeutics. In particular, the compositions described herein are used in methods of treating lung disease and injury including but not limited to acute respiratory distress syndrome (ARDS), COVID infection, cytokine storms, sepsis and related conditions. These compositions include, but are not limited to, peptide variants and compositions that inhibit activity of a receptor complex formed by triggering receptors expressed on myeloid cells (TREM; i.e., TREM-1, TREM-2, TREM-3 or TREM-4) and DNAX activation protein of 12 kDa (DAP 12).

### CROSS-REFERENCE TO RELATED APPLICATIONS

This application claims priority to United States Provisional Applications No. 63/190,497 filed on May 19, 2021 which is incorporated herein by reference in their entireties and for all purposes.

### FIELD OF THE INVENTION

The present invention is related to the field of pulmonary therapeutics. In particular, the compositions described herein are used in methods of treating lung disease and injury including, but not limited, to lung injuries caused by ionizing radiation, chemicals, bacteria and viruses, acute lung injury (ALI), acute respiratory distress syndrome (ARDS), chronic lung injury, COVID infection, sepsis and related conditions. These compositions include, but are not limited to, peptide variants and compositions that inhibit activity of receptor complexes formed by triggering receptors expressed on myeloid cells (TREM; i.e., TREM-1, TREM-2, TREM-3 or TREM-4) and DNAX activation protein of 12 kDa (DAP12).

### BACKGROUND OF THE INVENTION

TREM receptors are a family of cell-surface molecules that control inflammation, bone homeostasis, neurological development and blood coagulation. TREM-1 and TREM-2, the best-characterized receptors so far, play divergent roles in multiple diseases and disorders. For downstream signal transduction, TREMs are coupled to the immunoreceptor tyrosine-based activation motif (ITAM)-containing adaptor, DAP12.

TREM-1 amplifies the inflammatory response (Colonna 2003) and is upregulated under inflammatory conditions including cancer (Wang et al. 2004, Mehta et al. 2013, Sigalov 2014b) and lung diseases (Gibot 2006a, Yuan et al. 2016, Sadikot et al. 2017). TREM-1/DAP-12 receptor complex activation enhances release of multiple cytokines including monocyte chemoattractant protein-1 (MCP-1), tumor necrosis factor-a (TNFa), interleukin-1a (IL-1a), IL-1b, IL-6 and colony stimulating factor 1 (referred to herein as CSF1; also referred to in the art as macrophage colony stimulating factor, M-CSF) (Schenk et al. 2007, Lagler et al. 2009, Sigalov 2014b).

TREM-2 is expressed in a subgroup of myeloid cells including dendritic cells, granulocytes, and tissue-specific macrophages like tumor-associated macrophages (TAMs), osteoclasts, Kuppfer cells and alveolar macrophages (Gratuze et al. 2018, Cheng et al. 2021, Qiu et al. 2021). TREM-2 is known to promote macrophage survival and lung disease after respiratory viral infection and play a role in the development of chronic lung disease (Wu et al. 2015).

There are currently no effective pharmacologic therapies for treatment or prevention of most lung-related disorders and injuries induced by bacteria, viruses, chemical agents, radiation including but not limited to ionizing radiation, and other agents. While inhibition of fibrin formation mitigates injury in some preclinical models of ARDS, anticoagulation therapies in humans do not attenuate ARDS and may even increase mortality. Protective lung ventilator strategies remain the mainstay of available treatment options. Due to the significant morbidity and mortality associated with lung-related diseases and disorders and the lack of effective treatment options, new therapeutic agents and new treatment methods for the treatment and prevention of these diseases and disorders.

There is an unmet need in the art which provide novel targeted therapeutic agents useful in the treatment of ARDS and other lung diseases and injuries and methods of treatment for lung diseases and injuries and conditions related thereto.

### SUMMARY OF THE INVENTION

The present invention is related to the field of pulmonary therapeutics. In particular, the compositions described herein are used in methods of treating lung disease and injury including but not limited to ARDS, COVID infection, acute radiation syndrome (ARS) and delayed effects of radiation exposure (DEARE), lung injuries caused by radiation, chemicals, cytokine storms, bacteria, viruses, sepsis and related conditions. These compositions include, but are not limited to, peptide variants and compositions that inhibit activity of receptor complexes including but not limited to the receptor complexes formed by a TREM receptor nd DAP12 on myeloid cells including but not limited to TREM-1/DAP12 and TREM-2/DAP12 receptor complexes. Other compositions of the present invention include, but are not limited to, combinatorial peptide variants and compositions that concurrently inhibit activity of two or more cell receptors of the multichain immune recognition receptor (MIRR) family. In one embodiment, said cell receptors include but are not limited to, TREM receptors, T cell receptor, natural killer cell receptors, glycoprotein VI receptor, B cell receptor, and others.

The compositions described herein can also be used in methods of treating other inflammation-associated diseases and conditions including but not limited to cancer, cardiovascular diseases, retinopathy, autoimmune diseases, spinal cord injuries, etc.

In one embodiment, the present invention contemplates a peptide comprising an amino acid sequence having the general formula of R1-AA1-AA2-A1-A2-B-C-D1-D2-E-EE1-EE2-R2, wherein: R1 is absent or is selected from the group consisting of N-terminal sugar conjugate and N-terminal lipid conjugate; AA1 is absent or is selected from the group consisting of Arg, Arg-Arg, Arg-Arg-Arg and Arg-Arg-Arg-Arg; AA2 is absent or is selected from the group consisting of Lys, Lys-Lys, Lys-Lys-Lys and Lys-Lys-Lys-Lys; A1 is an amino acid selected from the group consisting of Pro, Cys, Leu, Ala, Val, Ile, Met, Trp, Gly and Phe, a two amino acid peptide, a three amino acid peptide, a four amino acid peptide, a five amino acid peptide, a six amino acid peptide and a seven amino acid peptide, said peptide consisting of Pro, Cys, Leu, Ala, Val, Ile, Met, Trp, Gly and Phe in any combination; A2 is absent or is a positively charged amino acid selected from the group comprising Arg, Lys and His; B is selected from the group consisting of Pro, Cys, Leu, Ala, Val, Ile, Met, Trp, Gly and Phe, a two amino acid peptide and a three amino acid peptide, said peptide consisting of Pro, Cys, Leu, Ala, Val, Ile, Met, Trp, Gly and Phe in any combination; C is a positively charged amino acid selected from the group comprising Arg, Lys and His; D1 is selected from the group consisting of Pro, Cys, Leu, Ala, Val, Ile, Met, Trp, Gly and Phe, a two amino acid peptide and a three amino acid peptide, said peptide consisting of Pro, Cys, Leu, Ala, Val, Ile, Met, Trp, Gly and Phe in any combination; D2 is absent or is a positively charged amino acid selected from the group comprising Arg, Lys and His; E is an amino acid selected from the group consisting of Pro, Cys, Leu, Ala, Val, Ile, Met, Trp, Gly and Phe, a two amino acid peptide, a three amino acid peptide, a four amino acid peptide, a five amino acid peptide, a six amino acid peptide and a seven amino acid peptide, said peptide consisting of Pro, Cys, Leu, Ala, Val, Ile, Met, Trp, Gly and Phe in any combination; EE1 is absent or is selected from the group consisting of Arg, Arg-Arg, Arg-Arg-Arg and Arg-Arg-Arg-Arg; EE2 is absent or is selected from the group consisting of Lys, Lys-Lys, Lys-Lys-Lys and Lys-Lys-Lys-Lys; and R2 is absent or is C-terminal lipid conjugate. In one embodiment, the distance between A2 and C1 is one to three amino acid residues. In one embodiment, the distance between C1 and D2 is one to three amino acid residues. In one embodiment, the N-terminal sugar conjugate is 1-amino-glucose succinate. In one embodiment, the N-terminal lipid conjugate includes, but is not limited to 2-aminododecanoate and/or myristoylate conjugates. In one embodiment, the C-terminal lipid conjugate includes but is not limited to Gly-Tris-monopalmitate, Gly-Tris-dipalmitate and/or Gly-Tris-tripalmitate conjugates.

In one embodiment, the peptide is attached to a carrier molecule. In one embodiment, the peptide is conjugated at a free amine group with a polyalkylene glycol. In one embodiment, the polyalkylene glycol is polyethylene glycol. In one embodiment, the one or more amino acids is a D-amino acid. In one embodiment, the peptide is a cyclic peptide. In one embodiment, the peptide is a cyclic dimer peptide. In one embodiment, the peptide is a dimer peptide.

In one embodiment, the present invention contemplates a method, comprising: a) providing; i) a patient exhibiting at least one symptom of a lung disease; and ii) a pharmaceutically acceptable composition comprising a TREM-2 inhibitor; and b) administering said composition to said patient such that said at least one symptom is reduced. In one embodiment, the TREM-2 inhibitor comprises a peptide with an amino acid sequence having the general formula of R1-AA1-AA2-A1-A2-B-C-D1-D2-E-EE1-EE2-R2. In one embodiment, the pharmaceutically acceptable composition further comprises a lipopeptide/lipoprotein complex. In one embodiment, the lipopeptide/lipoprotein complex further comprises a complex of a peptide selected from the group consisting of IFLIKILAAPLGEEMRDRARAHVDALRTHLA and IFLIKILAAPYLDDFQKKWQEEMELYRQKVE. In one embodiment, the methionine residues of said IFLIKILAAPLGEEMRDRARAHVDALRTHLA and said IFLIKILAAPYLDDFQKKWQEEMELYRQKVE are sulfoxidized. In one embodiment, the lipopeptide/lipoprotein complex comprises a lipid selected from the group consisting of phospholipid, cholesterol. cholesteryl oleate and any combination thereof. In one embodiment, where said lipopeptide/lipoprotein complex comprises a lipid selected from the group consisting of 1-palmitoyl-2-oleoyl-sn-glycero-3-phosphocholine, 1,2-dipalmitoyl-sn-glycero-3-phosphocholine, egg yolk L-a-phosphatidylcholine, soy L-a-phosphatidylcholine and any combination thereof. In one embodiment, the at least one symptom is selected from the group consisting of shortness of breath, inflammation of lung parenchyma, infiltration of neutrophils into pulmonary airspaces, oxidative stress, disruption of the endothelial barriers, disruption of the epithelial barriers, pulmonary epithelial lining damage, lung fibrosis, progressive hypoxemia and dyspnea. In one embodiment, the lung disease is an acute respiratory distress syndrome. In one embodiment, the acute respiratory distress syndrome is induced by a medical condition selected from the group consisting of sepsis, bacterial pneumonia, viral pneumonia, inhalation of harmful substances, major injury, burns, blood transfusions, near drowning, aspiration of gastric contents, pancreatitis, intravenous drug use, abdominal trauma and chronic alcoholism. In one embodiment, the lung disease is a chemical injury. In one embodiment, the chemical injury is selected from the group comprising a mustard gas injury, a phosgene injury and a chlorine injury. In one embodiment, the lung disease is a radiation lung injury. In one embodiment, the radiation injury is an ionizing radiation injury.

In one embodiment, the present invention contemplates a method, comprising: a) providing; i) a patient exhibiting at least one symptom of a COVID 19 infection; and ii) a pharmaceutically acceptable composition comprising a TREM-2 inhibitor; and b) administering said composition to said patient such that said COVID 19 infection is reduced. In one embodiment, the TREM-2 inhibitor comprises a peptide with an amino acid sequence having the general formula of R1-AA1-AA2-A1-A2-B-C-D1-D2-E-EE1-EE2-R2. In one embodiment, the peptide is: IFLIKILAAPLGEEMRDRARAHVDALRTHLA or IFLIKILAAPYLDDFQKKWQEEMELYRQKVE. In one embodiment, the patient further exhibits a cytokine storm. In one embodiment, the administering further reduces said cytokine storm. In one embodiment, the pharmaceutically acceptable composition further comprises a lipopeptide/lipoprotein complex.

In one embodiment, the present invention contemplates a method, comprising: a) providing; i) a patient exhibiting at least one symptom of a lung injury and condition; and ii) a pharmaceutically acceptable composition comprising a TREM-1 and/or TREM-2 inhibitor peptide and/or combinatorial TREM-1 and TREM-2 concurrent inhibitor peptide; and b) administering the composition to the patient such that the at least one symptom is reduced. In one embodiment, said TREM-1 inhibitory peptide comprises an amino acid sequence GFLSKSLVF. In one embodiment, said TREM-2 inhibitor peptide comprises an amino acid sequence IFLIKILAA. In one embodiment, said combinatorial TREM-1 and TREM-2 concurrent inhibitor peptide comprises an amino acid sequence selected from the group consisting of GFLSKSLVFIFLIKILAA (GA-18), IFLIKILAAGFLSKSLVF, RGFFRGGIFLIKILAA, IFLIKILAARGFFRGG, LQEEDAGEYGCMIFLIKILAA, IFLIKILAALQEEDAGEYGCM, LQVTDSGLYRCVIYHPPIFLIKILAA, and IFLIKILAALQEEDAGEYGCM. In one embodiment, lung injury and condition are induced by radiation, chemicals, bacteria or viruses.

In one embodiment, said radiation is ionizing radiation. In one embodiment, said chemicals include but are not limited to sulfur mustard (SM), phosgene or chlorine. In one embodiment, said bacteria is Bordetella pertussis (B.pertussis). In one embodiment, said viruses are severe acute respiratory syndrome (SARS) coronaviruses 1 and 2 (SARS-CoV-1 and SARS-CoV-2, respectively). In one embodiment, lung injury and condition include, but are not limited to, ARDS. In one embodiment, the at least one symptom of ARDS is shortness of breath. In one embodiment, the at least one symptom of ARDS is selected from the group consisting of inflammation of lung parenchyma, infiltration of neutrophils into pulmonary airspaces, oxidative stress, disruption of the endothelial and epithelial barriers, pulmonary epithelial lining damage, lung fibrosis, progressive hypoxemia and dyspnea. In one embodiment, ARDS is induced by a medical condition selected from the group consisting of sepsis, bacterial pneumonia, viral pneumonia, inhalation of harmful substances, major injury, burns, blood transfusions, near drowning, aspiration of gastric contents, pancreatitis, intravenous drug use, abdominal trauma and chronic alcoholism. In one embodiment, the pharmaceutically acceptable composition further comprises a lipoprotein complex.

In one embodiment, the present invention contemplates a method, comprising a) providing; i) a patient exhibiting at least one symptom of a COVID 19 infection; and ii) a pharmaceutically acceptable composition comprising a TREM-1 and/or TREM-2 inhibitor peptide and/or combinatorial TREM-1 and TREM-2 concurrent inhibitor peptide; and b) administering the composition to the patient such that the COVID 19 infection is reduced. In one embodiment, said TREM-1 inhibitor peptide comprises an amino acid sequence GFLSKSLVF. In one embodiment, said TREM-2 inhibitor peptide comprises an amino acid sequence IFLIKILAA. In one embodiment, said combinatorial TREM-1 and TREM-2 concurrent inhibitor peptide comprises an amino acid sequence selected from the group consisting of GFLSKSLVFIFLIKILAA (GA-18), IFLIKILAAGFLSKSLVF, RGFFRGGIFLIKILAA, IFLIKILAARGFFRGG, LQEEDAGEYGCMIFLIKILAA, IFLIKILAALQEEDAGEYGCM, LQVTDSGLYRCVIYHPPIFLIKILAA, and IFLIKTLAALQEEDAGEYGCM. In one embodiment, the patient further exhibits a cytokine storm. In one embodiment, said administering further reduces the cytokine storm. In one embodiment, the pharmaceutically acceptable composition further comprises a lipoprotein/lipopeptide complex.

In one embodiment, the present invention contemplates a method, comprising a) providing; i) a patient exhibiting at least one symptom of a chemical lung injury; and ii) a pharmaceutically acceptable composition comprising a TREM-1 and/or TREM-2 inhibitor peptide and/or combinatorial TREM-1 and TREM-2 concurrent inhibitor peptide; and b) administering the composition to the patient such that the chemical lung injury is reduced. In one embodiment, said TREM-1 inhibitor peptide comprises an amino acid sequence GFLSKSLVF. In one embodiment, said TREM-2 inhibitor peptide comprises an amino acid sequence IFLIKILAA. In one embodiment, said combinatorial TREM-1 and TREM-2 concurrent inhibitor peptide comprises an amino acid sequence selected from the group consisting of GFLSKSLVFIFLIKILAA (GA-18), IFLIKILAAGFLSKSLVF, RGFFRGGIFLIKILAA, IFLIKILAARGFFRGG, LQEEDAGEYGCMIFLIKILAA, IFLIKILAALQEEDAGEYGCM, LQVTDSGLYRCVIYHPPIFLIKILAA, and IFLIKTLAALQEEDAGEYGCM. In one embodiment, the chemical injury is a SM gas injury. In one embodiment, the chemical injury is a phosgene injury. In one embodiment, the chemical injury is a chlorine injury. In one embodiment, the pharmaceutically acceptable composition further comprises a lipoprotein/lipopeptide complex.

In one embodiment, the present invention contemplates a method, comprising a) providing; i) a patient exhibiting at least one symptom of a radiation lung injury; and ii) a pharmaceutically acceptable composition comprising a TREM-1 and/or TREM-2 inhibitor peptide and/or combinatorial TREM-1 and TREM-2 inhibitor concurrent peptide; and b) administering the composition to the patient such that the radiation lung injury is reduced. In one embodiment, said TREM-1 inhibitor peptide comprises an amino acid sequence GFLSKSLVF. In one embodiment, said TREM-2 inhibitory peptide comprises an amino acid sequence IFLIKILAA. In one embodiment, said combinatorial TREM-1 and TREM-2 concurrent inhibitor peptide comprises an amino acid sequence selected from the group consisting of GFLSKSLVFIFLIKILAA (GA-18), IFLIKILAAGFLSKSLVF, RGFFRGGIFLIKILAA, IFLIKILAARGFFRGG, LQEEDAGEYGCMIFLIKILAA, IFLIKILAALQEEDAGEYGCM, LQVTDSGLYRCVIYHPPIFLIKILAA, and IFLIKILAALQEEDAGEYGCM. In one embodiment, said radiation injury is an ionizing radiation injury. In one embodiment, the pharmaceutically acceptable composition further comprises a lipoprotein/lipopeptide complex.

In one embodiment, the present invention contemplates a method, comprising a) providing; i) a patient exhibiting at least one symptom of bacterial infection-induced lung injury; and ii) a pharmaceutically acceptable composition comprising a TREM-1 and/or TREM-2 inhibitor peptide and/or combinatorial TREM-1 and TREM-2 concurrent inhibitor peptide; and b) administering the composition to the patient such that the bacterial infection-induced lung injury is reduced. In one embodiment, said TREM-1 inhibitor peptide comprises an amino acid sequence GFLSKSLVF. In one embodiment, said TREM-2 inhibitor peptide comprises an amino acid sequence IFLIKILAA. In one embodiment, said combinatorial TREM-1 and TREM-2 concurrent inhibitor peptide comprises an amino acid sequence selected from the group consisting of GFLSKSLVFIFLIKILAA (GA-18), IFLIKILAAGFLSKSLVF, RGFFRGGIFLIKILAA, IFLIKILAARGFFRGG, LQEEDAGEYGCMIFLIKILAA, IFLIKILAALQEEDAGEYGCM, LQVTDSGLYRCVIYHPPIFLIKILAA, and IFLIKILAALQEEDAGEYGCM. In one embodiment, said bacterial infection is B.pertussis infection. In one embodiment, the bacterial infection-induced lung injury is a whooping cough (pertussis). In one embodiment, the pharmaceutically acceptable composition further comprises a lipoprotein/lipopeptide complex.

In one embodiment, the present invention contemplates, a method for treating an inflammatory condition of the lungs, disease of the lungs or lung injury in a subject in need thereof, said method comprising administering to said patient an amount of at least one TREM-1 and/or TREM-2 inhibitor and/or combinatorial TREM-1 and TREM-2 concurrent inhibitor that is effective for inhibiting the TREM-1/DAP-12 or TREM-2/DAP12 signaling pathways or both simultaneously, respectively, and suppressing inflammatory response, or combinations thereof, and wherein the subject is selected from the group consisting of human and animal. In one embodiment, the method further comprises administering the amount of the TREM-1 and/or TREM-2 inhibitor and/or combinatorial TREM-1 and TREM-2 concurrent inhibitor together with a pharmaceutically acceptable excipient, carrier, diluents, or a combination thereof. In one embodiment, the inflammatory condition of the lungs, disease of the lungs or lung injury is selected from the group consisting of ARDS, including occurrences of ARDS caused by at least in part, by bacterial pneumonia, viral pneumonia including upper respiratory tract infections such as SARS (including but not limited to SARS CoV and SARS CoV-2) and MERS, sepsis, head injury, chest injury, burns, blood transfusions, near drowning, aspiration of gastric contents, pancreatitis, intravenous drug use, abdominal trauma, acute lung injury, pulmonary fibrosis (idiopathic), acute lung injury (ALI), ventilator-induced lung injury (VILI), bleomycin-induced pulmonary fibrosis, mechanical ventilator-induced lung injury, chronic obstructive pulmonary disease (COPD), chronic bronchitis, emphysema, bronchiolitis obliterans after lung transplantation and lung transplantation-induced acute graft dysfunction, including treatment, prevention or prevention of progression of primary graft failure, ischemia-reperfusion injury, reperfusion injury, reperfusion edema, allograft dysfunction, pulmonary reimplantation response, bronchiolitis obliterans after lung transplantation and/or primary graft dysfunction (PGD) after organ transplantation, in particular in lung transplantation, or a combination of any of the foregoing. In one embodiment, the inflammatory condition of the lungs, disease of the lungs or lung injury is coronavirus disease (COVID-19).

In one embodiment, the at least one said TREM-1 inhibitor comprises a variant TREM-1 inhibitor peptide sequence derived from transmembrane domain sequences of human or animal TREM-1 and/or its signaling subunit, DAP-12, thereof. In one embodiment, the at least one said TREM-2 inhibitor comprises a variant TREM-2 inhibitor peptide sequence derived from transmembrane domain sequence of human or animal TREM-2. In one embodiment, a combination of TREM-1 inhibitors is administered to the subject. In one embodiment, a combination of TREM-2 inhibitors is administered to the subject. In one embodiment, a combination of TREM-1 and TREM-2 inhibitors is administered to the subject. In one embodiment, combinatorial TREM-1 and TREM-2 concurrent inhibitor comprises a combinatorial variant TREM-1 and TREM-2 inhibitor peptide sequence derived from transmembrane domain sequences of TREM-1 and TREM-2, or combinations thereof. In one embodiment, said TREM-1 and TREM-2 are human or animal TREM-1 and TREM-2. In certain embodiments, combinatorial inhibitors that target two or more MIRRs simultaneously comprise combinatorial variant MIRR inhibitor peptide sequences derived from transmembrane domain sequences of MIRRs using the SCHOOL model (Sigalov 2010b, Sigalov 2010a). In certain embodiments, said combinatorial variant MIRR inhibitor peptide sequences can be used for treating and imaging any diseases and condition in which these MIRRs are involved. In certain embodiments, said MIRRs are human or animal MIRRs.

In one embodiment, the at least one said TREM-1 inhibitor comprises an isolated antibody of fragment thereof capable of binding to Peptidoglycan recognition protein 1 (PGLYRP1) and reducing PGLYRP1-mediated TREM-1 activity. In one embodiment, the at least one said TREM-1 inhibitor comprises an isolated antibody of fragment thereof capable of binding to TREM-1 and reducing TREM-1 activity. In one embodiment, the at least one said TREM-2 inhibitor comprises an isolated antibody of fragment thereof capable of binding to TREM-2 and reducing TREM-2 activity.

In one embodiment, the present invention contemplated a method of predicting the efficacy of TREM-1-targeted therapies in an individual in need thereof, by: (a) obtaining a biological sample from the individual; (b) determining the number of myeloid cells in the biological sample; (c) determining the expression levels of TREM-1 in the cells contained within the biological sample; (d) measuring the level of soluble form of the human TREM-1 receptor in the biological sample. In one embodiment, the step of measuring the level of the soluble form of the human TREM-1 receptor comprises the steps of: (a) contacting said biological sample with a compound capable of binding the soluble form of the human TREM-1 receptor; (b) detecting the level of the soluble form of the human TREM-1 receptor present in the sample by observing the level of binding between said compound and the soluble form of the human TREM-1 receptor. In one embodiment, the method further comprises the steps of measuring the level of the soluble form of the human TREM-1 receptor in a second or further sample from said subject, the first and second or further samples being obtained at different times; and comparing the levels in the samples to indicate the progression or remission of the proliferative disease.

In one embodiment, the present invention contemplated a method of predicting the efficacy of TREM-2-targeted therapies in an individual in need thereof, by: (a) obtaining a biological sample from the individual; (b) determining the number of myeloid cells in the biological sample; (c) determining the expression levels of TREM-2 in the cells contained within the biological sample; (d) measuring the level of soluble form of the human TREM-2 receptor in the biological sample. In one embodiment, the step of measuring the level of the soluble form of the human TREM-2 receptor comprises the steps of: (a) contacting said biological sample with a compound capable of binding the soluble form of the human TREM-2 receptor; (b) detecting the level of the soluble form of the human TREM-2 receptor present in the sample by observing the level of binding between said compound and the soluble form of the human TREM-2 receptor.

In one embodiment, the method further comprises the steps of measuring the level of the soluble form of the human TREM-2 receptor in a second or further sample from said subject, the first and second or further samples being obtained at different times; and comparing the levels in the samples to indicate the progression or remission of the proliferative disease.

In one embodiment, the sample is selected from the group consisting of whole blood, blood serum, blood, plasma, urine, bronchoalveolar lavage fluid (BALF) and synovial liquid. In one embodiment, the sample is from synovial fluid. In one embodiment, the sample is from blood serum or blood plasma. In one embodiment, the sample is a human sample. In one embodiment, the compound specifically binds the soluble form of the human TREM-1 receptor.

In one embodiment, the compound capable of binding the soluble form of the human TREM-1 receptor is an antibody raised against all or part of the TREM-1 receptor. In one embodiment, the level of soluble form of the human TREM-1 receptor is measured by an immunochemical technique. In one embodiment, the method further comprises measuring the level of TREM-1 ligand in one or more biological samples obtained from said subject. In one embodiment, the compound specifically binds the soluble form of the human TREM-2 receptor. In one embodiment, the compound capable of binding the soluble form of the human TREM-2 receptor is an antibody raised against all or part of the TREM-2 receptor. In one embodiment, the level of soluble form of the human TREM-2 receptor is measured by an immunochemical technique. In one embodiment, the method further comprises measuring the level of TREM-2 ligand in one or more biological samples obtained from said subject.

In one embodiment, the present invention contemplates a method of imaging a TREM-1-expressing cell-related condition, comprising a) providing; i) a patient having at least one symptom of a disease or condition in which TREM-1-expressing cells are involved or recruited, and ii) a labeled probe, wherein the probe has an affinity for TREM-1 and is labeled with an imaging probe; b) administering said composition to said patient in a detectably effective amount c) imaging at least a portion of the patient; and d) detecting the labeled probe, wherein the location of the labeled probe corresponds to at least one symptom of the TREM-1-expressing cell-related condition. In one embodiment, the imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), T1201, K42, In111, Fe59, Tc99m, Cr51, Ga67, Ga68, Cu64, Rb82, Mo99, Dy165, Fluorescein, Carboxyfluorescein, Calcein, F18, Xe133, I125, I131, I123, P32, C11, N13, O15, Br76, Kr81, Diatrizoate, Metrizoate, Isopaque, Ioxaglate, Iopamidol, Iohexol, Iodixanol.

In one embodiment, the present invention contemplates a method of imaging a TREM-2-expressing cell-related condition, comprising a) providing; i) a patient having at least one symptom of a disease or condition in which TREM-2-expressing cells are involved or recruited, and ii) a labeled probe, wherein the probe has an affinity for TREM-2 and is labeled with an imaging probe; b) administering said composition to said patient in a detectably effective amount c) imaging at least a portion of the patient; and d) detecting the labeled probe, wherein the location of the labeled probe corresponds to at least one symptom of the TREM-2-expressing cell-related condition. In one embodiment, the imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), T1201, K42, In111, Fe59, Tc99m, Cr51, Ga67, Ga68, Cu64, Rb82, Mo99, Dy165, Fluorescein, Carboxyfluorescein, Calcein, F18, Xe133, I125, I131, I123, P32, C11, N13, O15, Br76, Kr81, Diatrizoate, Metrizoate, Isopaque, Ioxaglate, Iopamidol, Iohexol, Iodixanol.

In one embodiment, the present invention contemplates a method of imaging TREM-1- and TREM-2-expressing cell-related condition, comprising a) providing; i) a patient having at least one symptom of a disease or condition in which TREM-1- and TREM-2-expressing cells are involved or recruited, and ii) a labeled probe, wherein the probe has an affinity for both TREM-1 and TREM-2 and is labeled with an imaging probe; b) administering said composition to said patient in a detectably effective amount c) imaging at least a portion of the patient; and d) detecting the labeled probe, wherein the location of the labeled probe corresponds to at least one symptom of the TREM-1- and TREM-2-expressing cell-related condition. In one embodiment, the imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), T1201, K42, In111, Fe59, Tc99m, Cr51, Ga67, Ga68, Cu64, Rb82, Mo99, Dy165, Fluorescein, Carboxyfluorescein, Calcein, F18, Xe133, 1125, 1131, 1123, P32, C11, N13, O15, Br76, Kr81, Diatrizoate, Metrizoate, Isopaque, Ioxaglate, Iopamidol, Iohexol, Iodixanol.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Exemplary techniques for chemical syntheses, chemical analyses, pharmaceutical preparation, formulation, and delivery, and treatment of patients are known in the art. As utilized in accordance with the present disclosure, the following terms, unless otherwise indicated, shall be understood to have the following meanings:

The terms “lung diseases”, “lung disorders” or “lung injury” referred to herein include, but are not limited to, acute respiratory distress syndrome (ARDS), including occurrences of ARDS caused by upper respiratory tract bacterial and viral infections such as Bordetella pertussis, Streptococcus pneumoniae, Haemophilus species, Staphylococcus aureus, Mycobacterium tuberculosis, severe acute respiratory syndrome coronaviruses (SARS-CoVs, including but not limited to SARS-CoV-1 and SARS-CoV-2) and Middle East respiratory syndrome (MERS), acute lung injury (ALI), chronic lung injury, pulmonary fibrosis (idiopathic), ventilator-induced lung injury (VILI), radiation-induced lung injury and delayed effects of radiation exposure (DEARE), lung injury caused by chemicals affecting the respiratory tract, bleomycin-induced pulmonary fibrosis, mechanical ventilator-induced lung injury, chronic obstructive pulmonary disease (COPD), chronic bronchitis, emphysema, bronchiolitis obliterans after lung transplantation and lung transplantation-induced acute graft dysfunction, including treatment, prevention or prevention of progression of primary graft failure, ischemia-reperfusion injury, reperfusion injury, reperfusion edema, allograft dysfunction, pulmonary reimplantation response, bronchiolitis obliterans after lung transplantation and/or primary graft dysfunction (PGD) after organ transplantation, in particular in lung transplantation. Secondary conditions of these lung disorders also involve cytokine storms and sepsis.

The terms “APOA1_HUMAN”, “Apolipoprotein A-I”, “Apolipoprotein A-1”, “APOA1”, “ApoA-I”, “Apo-AI”, “ApoA-1”, “apo-A1”, “apoA-1” and “Apo-A1” refer to the naturally occurring human protein listed in the UniProt Knowledgebase (UniProtKB, www.uniprot.org) under the name “APOA1_HUMAN”. The protein amino acid sequence can be found under the entry UniProt KB/Swiss-Prot P02647 (www.uniprot.org/uniprot/P02647). The terms “APOA2_HUMAN”, “Apolipoprotein A-II”, 20 Apolipoprotein A-2”, “APOA2”, “ApoA-II”, “Apo-AII”, “ApoA-2”, “apo-A2”, “apoA-2” and “Apo-A2” refer to the naturally occurring human protein listed in the UniProt Knowledgebase (UniProtKB, www.uniprot.org) under the name “APOA2_HUMAN”. The protein amino acid sequence can be found under the entry UniProt KB/Swiss-Prot P02652 (http://www.uniprot.org/uniprot/PO2652). The terms “TREM1_HUMAN”, “TREM-1 receptor”, “TREM-1 receptor subunit”, “TREM-1 subunit”, and “TREM-1 recognition subunit” refer to the naturally occurring human protein listed in the UniProt Knowledgebase (UniProtKB, www.uniprot.org) under the name “TREM1_HUMAN”. The protein amino acid sequence can be found under the entry UniProt KB/Swiss-Prot Q9NP99.

The term, “amino acid domain”, as used herein refers to is a contiguous polymer of at least two (2) amino acids joined by peptide bond(s). The domain may be joined to another amino acid or amino acid domain by one or more peptide bonds. An amino acid domain can constitute at least two amino acids at the N-terminus or C-terminus of a peptide or can constitute at least two amino acids in the middle of a peptide.

As used herein, a “peptide” and “polypeptide” comprises a string of at least two amino acids linked together by peptide bonds. A peptide generally represents a string of between approximately 2 and 200 amino acids, more typically between approximately 6 and 64 amino acids. Peptide may refer to an individual peptide or a collection of peptides. Peptides typically contain only natural amino acids, although non-natural amino acids (i.e., compositions that do not occur in nature but that can be incorporated into a polypeptide chain and/or amino acid analogs as are known in the art may alternatively be employed. In particular, D-amino acids may be used.

The term, “peptide sequence”, or “amino acid sequence”, as used herein refers to an order in which amino acid residues, connected by peptide bonds, lie in the chain in peptides. The sequence is generally reported from the N-terminal end containing free amino group to the C-terminal end containing free carboxyl group. A “peptide sequence” is often called “protein sequence” if it represents the primary structure of a protein.

As used herein, the term “aptamer” or “specifically binding oligonucleotide” refers to an oligonucleotide that is capable of forming a complex with an intended target substance.

The term “modified peptide”, as used herein refers to describe chemically or enzymatically, or chemically and enzymatically modified oligopeptides, oligopseudopeptides, polypeptides, and pseudopolypeptides (synthetic or otherwise derived), regardless of the nature of the chemical and/or enzymatic modification.

The term “pseudopeptide” refers to a peptide where one or more peptide bonds are replaced by non-amido bonds such as ester or one or more amino acids are replaced by amino acid analogs.

The term “peptides” refers not only to those comprised of all natural amino acids, but also to those which contain unnatural amino acids or other non-coded structural units including pseudopeptides. “Modified peptides” have utility in many biomedical applications because of their increased stability against in vivo degradation, superior pharmacokinetics, and altered immunogenicity compared to their native counterparts.

The term “modified peptides,” as used herein, also includes oxidized peptides.

The term “oxidized peptide” refers to a peptide in which at least one amino acid residue is oxidized.

The term “oxidation status” refers to a metric of the extent to which specific amino acid residues are replaced by corresponding oxidized amino acid residues in a peptide.

The term “extent of oxidation” refers to the degree to which potentially oxidizable amino acids in a peptide have undergone oxidation. For example, if the peptide contains a single tyrosine residue which is potentially oxidized to 3-chlorotyrosine, then an increase in mass of about thirty-four (34) Daltons (i.e., the approximate difference in mass between chlorine and hydrogen) indicates oxidation of tyrosine to 3-chlorotyrosine. Similarly, if the peptide contains a single methionine residue which is potentially oxidized to methionine sulfoxide, then an increase in mass of sixteen (16) Daltons (i.e., the difference in mass between methionine and methionine containing one extra oxygen) indicates oxidation of methionine to methionine sulfoxides. The oxidation status can be measured by metrics known to the arts of protein and peptide chemistry including, without limitation, assay of the number of oxidized residues, mass spectral peak intensity, mass spectral integrated area, and the like as disclosed in U.S. Pat. Nos. 8,114,613 and 8,338,110 (both herein incorporated by reference). In some embodiments of any of the aspects provided herein, oxidation status is reported as a percentage, wherein 0% refers to no oxidation and 100% refers to complete oxidation of potentially oxidizable amino acid residues within apo A-I or apo A-II peptide fragments.

The term “potentially subject to oxidation,” “potentially oxidizable amino acid residues”, and the like refer to an amino acid which can undergo oxidation, for example by nitration or chlorination.

A “biologically active peptide motif” is a peptide that induces a phenotypic response or change in an appropriate cell type when the cell is contacted with the peptide. The peptide may be present either in isolated form or as part of a larger polypeptide or other molecule. The ability of the peptide to elicit the response may be determined, for example, by comparing the relevant parameter in the absence of the peptide (e.g., by mutating or removing the peptide when normally present within a larger polypeptide). Phenotypic responses or changes include, but are not limited to, enhancement of cell spreading, attachment, adhesion, proliferation, secretion of an extracellular matrix (ECM) molecule, or expression of a phenotype characteristic of a particular differentiated cell type.

As used herein, a “minimal biologically active sequence” refers to the minimum length of a sequence of a peptide which has a specific biological function. For example, in one embodiment, the first and second amino acid domains of a TREM-1 or TREM-2 inhibitor peptides contains at least one minimal biologically active sequence. This minimal biologically active sequence is any length of sequence from a full length peptide sequence. Moreover, with the exception of the amino acids of the minimal biologically active sequence, the amino acids of any or both amino acid domain can be exchanged, added or removed according to the design of the molecule to adjust its overall hydrophilicity and/or net charge. In some embodiments, the minimal biologically active sequence refers to any one of the sequences provided herein and to the sequences disclosed in U.S. Pat. No. 8,513,185B2; U.S. Pat. Nos. 9,981,004; 8,513,185; US 2019/0117725; and US 2014/0154291; (all of which are incorporated herein by reference); and PCT/US2010/052117; PCT/US2019/046392; and WO 2020/036987.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “combinatorial peptide variant” or “combinatorial peptide sequence” or “combinatorial peptide” or “combinatorial concurrent inhibitor peptide sequence” as used herein refers to amino acid sequence that combines two or more “biologically active peptide motifs” or “minimal biologically active sequences”. Example is GFLSKSLVFIFLIKILAA (GA-18 peptide) that combines two biologically active peptide motifs (GFLSKSLVF and IFLIKILAA) to target the TREM-1/DAP12 and TREM-2/DAP12 receptor complexes simultaneously (concurrently).

The terms “lipopeptide complex” (or “lipoprotein complex”, LPC), “synthetic lipopeptide particle” (SLP), “lipopeptide nanoparticle” (LNP), or “recombinant high density lipoproteins” (rHDL) as used herein refer to complexes formed by peptide variants and compositions of the present invention and at least one lipid.

The term “encapsulation” as used herein refers to the enclosure of a molecule, such as trifunctional peptides and compositions of the present invention, inside the nanoparticle.

The term “incorporation” as used herein refers to imbibing or adsorbing the trifunctional peptides and compositions onto the nanoparticle.

The terms “reconstituted” and “recombinant” as used herein both refer to synthetic lipopeptide particles that represent both discoidal and spherical nanoparticles and mimic native high density lipoprotein (HDL) particles.

As used herein, “naturally occurring” means found in nature. A naturally occurring biomolecule is, in general, synthesized by an organism that is found in nature and is unmodified by the hand of man, or is a degradation product of such a molecule. A molecule that is synthesized by a process that involves the hand of man (e.g., through chemical synthesis not involving a living organism or through a process that involves a living organism that has been manipulated by the hand of man or is descended from such an organism) but that is identical to a molecule that is synthesized by an organism that is found in nature and is unmodified by the hand of man is also considered a naturally occurring molecule.

The term “site of interest” on a target as used herein is a site to which modified peptides and compositions of the present invention bind.

The term “target site”, as used herein, refers to sites/tissue areas of interest.

The terms “target cells” or “target tissues”, as used herein, refer to those cells or tissues, respectively that are intended to be targeted using the compositions of the present invention delivered in accord with the invention. Target cells or target tissues take up or link with the modified peptides and compositions of the invention. For example, the terms “target cells” or “target tissues” refer to those cells or tissues, respectively, that are intended to be treated using the compositions of the present invention delivered in accord with the invention. Target cells are cells in target tissue, and the target tissue includes, but is not limited to, vascular endothelial tissue, abnormal vascular walls of tumors, solid tumors, tumor-associated macrophages, and other tissues or cells related to cancer, inflammatory diseases, and the like. Further, target cells include virus-containing cells, and parasite containing cells. Also included among target cells are cells undergoing substantially more rapid division as compared to non-target cells. The term “target cells” also includes, but is not limited to, microorganisms such as bacteria, viruses, fungi, parasites, and infectious agents. Thus, the term “target cell” is not limited to living cells but also includes infectious organic particles such as viruses.

The term “target compositions” or “target biological components” include, but are not be limited to: toxins, peptides, polymers, and other compositions that may be selectively and specifically identified as an organic target that is intended to be visualized in imaging techniques using the compositions of the present invention.

The term “therapeutic agent” or “drug” as used herein refers to any compound or composition having preventive, therapeutic or diagnostic activity, primarily but not exclusively in the treatment of patients with myeloid cell-related diseases.

The term “immune cells” include neutrophils, eosinophils, basophils, mast cells, monocytes, macrophages, dendritic cells, natural killer cells, and lymphocytes (B cells and T cells).

The term “myeloid cells” include monocytes, macrophages, neutrophils, basophils, eosinophils, erythrocytes, and megakaryocytes to platelets.

The terms “macrophage-associated”, “macrophage-mediated”, and “macrophage-related diseases” include diseases associated with macrophages as disclosed in U.S. Pat. No. 8,916,167 (herein incorporated by reference).

The term “myeloid cell-mediated pathology” (or “myeloid cell-related pathologies”, or “myeloid cell-mediated disorder, or “myeloid cell-related disease”), as used herein, refers to any condition in which an inappropriate myeloid cell response is a component of the pathology. The term is intended to include both diseases directly mediated by myeloid cells, and also diseases in which an inappropriate myeloid cell response contributes to the production of abnormal antibodies, antibodies, as well as graft rejection.

The term “ligand-induced myeloid cell activation”, as used herein, refers to myeloid cell activation in response to the stimulation by the specific ligand.

The term “T cells” refers to a type of lymphocytes that can be distinguished from other lymphocytes by the presence of TCR on their cell surface.

The terms “T cell-associated”, “T cell-mediated”, and “T cell-related diseases” include diseases associated with T cells as disclosed in U.S. Pat. Nos. 10,538,558 and 10,138,278 (both of which are herein incorporated by reference).

The term “T cell-mediated pathology” (or “T cell-related pathologies”, or “T cell-mediated disorder, or “T cell-related disease”), as used herein, refers to any condition in which an inappropriate T cell response is a component of the pathology. The term is intended to include both diseases directly mediated by T cells, and also diseases in which an inappropriate T cell response contributes to the production of abnormal antibodies, antibodies, as well as graft rejection.

The term “stimulation”, as used herein, refers to a primary response induced by ligation of a cell surface moiety. For example, in the context of receptors, such stimulation entails the ligation of a receptor and a subsequent signal transduction event. With respect to stimulation of a myeloid cell, such stimulation refers to the ligation of a myeloid cell surface moiety that in one embodiment subsequently induces a signal transduction event, such as binding the cell surface receptor (e.g., MIRR family member). Further, the stimulation event may activate a cell and up-regulate or down-regulate expression or secretion of a molecule.

The term “ligand”, or “antigen”, as used herein, refers to a stimulating molecule that binds to a defined population of cells. The ligand may bind any cell surface moiety, such as a receptor, an antigenic determinant, or other binding site present on the target cell population. The ligand may be a protein, peptide, antibody and antibody fragments thereof, fusion proteins, synthetic molecule, an organic molecule (e.g., a small molecule), or the like. In the context of myeloid cell stimulation, the ligand (or antigen) binds the cell receptor (eg. MIRR family member) and this binding activates the cell.

The term “TREM receptor”, as used herein, refers to a member of TREM receptor family: TREM-1, TREM-2, TREM-3 and TREM-4.

The term “activation”, as used herein, refers to the state of a cell following sufficient cell surface moiety ligation to induce a noticeable biochemical or morphological change. In the context of myeloid cells, such activation, refers to the state of a myeloid cell that has been sufficiently stimulated to induce production of interleukin 8 (IL-8), IL-6, IL-1b, tumor necrosis factor alpha (TNF-alpha) and other cytokines and chemokines, differentiation of primary monocytes into immature dendritic cells, and enhancement of inflammatory responses to microbial products. Within the context of other cells (e.g., non-myeloid cells), this term infers either up or down regulation of a particular physicochemical process.

The term “inhibiting myeloid cell activation” (or “TREM-mediated cell activation”), as used herein, refers to the slowing of myeloid cell activation, as well as completely eliminating and/or preventing myeloid cell activation.

The term “inhibiting T cell activation” (or “TCR-mediated cell activation”), as used herein, refers to the slowing of T cell activation, as well as completely eliminating and/or preventing T cell activation.

The term “multichain immune recognition receptor” (or “MIRR”), as used herein, refers to those cell surface receptors in which extracellular binding and intracellular signaling domains are located on separate receptor subunits as described in (Sigalov 2010a). Examples of MIRRs include but are not limited to B cell receptor (BCR), C-type lectin receptor (CLR); dendritic cell immunoactivating receptor (DCAR), glycoprotein VI (GPVI), Ig-like transcript (ILT); killer cell Ig-like receptor (KIR), leukocyte Ig-like receptor (LTR), myeloid-associated Ig-like receptor (MAIR-II), myeloid DAP12-associating lectin 1 (MDL-1), novel immune-type receptor (NITR), natural killer cell receptor family (NKCRs: KIR2DS, NKG2D, NKp46, NKp44, NKp30, etc), signal regulatory protein (SIRP), T cell receptor (TCR), TREM receptor family.

The term “TCR”, as used herein, refers to the TCR-CD3 complex comprising two antigen-binding chains (TCR alpha, TCRa, and TCR beta, TCRb) non-covalently complexed to TCR zeta (TCRz), CD3 epsilon (CD3e), CD3 gamma (CD3g) and CD3 delta (CD3d) signaling chains (or subunits).

The term “treating a disease or condition” as used herein, refers to modulating immune cell activation. This includes, but is not limited to, decreasing cytokine production and differentiation of primary monocytes into immature dendritic cells and/or slowing myeloid cell activation, as well as completely eliminating and/or preventing myeloid cell activation. Myeloid cell-related diseases and/or conditions treatable by modulating myeloid cell activation include, but are not limited to, myeloid cell-related inflammatory conditions such as lung disease and injury, tissue/organ rejection. T cell-related diseases and/or conditions treatable by modulating T cell activation include, but are not limited to, T cell-related inflammatory conditions such as autoimmune diseases and disorders, tissue/organ rejection.

The term, “subject” or “patient”, as used herein, refers to any individual organism. For example, the organism may be a mammal such as a primate (i.e., for example, a human). Further, the organism may be a domesticated animal (i.e., for example, cats, dogs, etc.), livestock (i.e., for example, cattle, horses, pigs, sheep, goats, etc.), or a laboratory animal (i.e., for example, mouse, rabbit, rat, guinea pig, etc.).

The term “resistant,” when used in the context of resistance to a therapeutic agent, means a decreased response or lack of response to a standard dose of the therapeutic agent, relative to the subject's response to the standard dose of the therapeutic agent in the past, or relative to the expected response of a similar subject with a similar disorder to the standard dose of the therapeutic agent. Thus, in some embodiments, a subject may be resistant to therapeutic agent although the subject has not previously been given the therapeutic agent, or the subject may develop resistance to the therapeutic agent after having responded to the agent on one or more previous occasions.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “sample,” as used herein, refers to a composition that is obtained or derived from a subject that contains a cellular and/or other molecular entity that is to be characterized, quantitated, and/or identified, for example based on physical, biochemical, chemical and/or physiological characteristics.

The term “tissue sample” refers to a collection of similar cells obtained from a tissue of a subject. The source of the tissue sample may be solid tissue as from a fresh, frozen and/or preserved organ or tissue sample or biopsy or aspirate; blood or any blood constituents; bodily fluids such as bronchoalveolar lavage (BAL) (also known as bronchoalveolar washing) fluid (BALF), cerebral spinal fluid, amniotic fluid, peritoneal fluid, synovial fluid, or interstitial fluid; cells from any time in gestation or development of the subject. In some embodiments, a tissue sample is a synovial biopsy tissue sample and/or a synovial fluid sample. In some embodiments, a tissue sample is a synovial fluid sample. The tissue sample may also be primary or cultured cells or cell lines. Optionally, the tissue sample is obtained from a disease tissue/organ. The tissue sample may contain compounds that are not naturally intermixed with the tissue in nature such as preservatives, anticoagulants, buffers, fixatives, nutrients, antibiotics, or the like.

A “control sample” or “control tissue”, as used herein, refers to a sample, cell, or tissue obtained from a source known, or believed, not to be afflicted with the disease for which the subject is being treated.

The term “section”, as used herein in reference to a tissue sample means a part or piece of a tissue sample, such as a thin slice of tissue or cells cut from a solid tissue sample.

The term, “therapeutically effective amount”, “therapeutically effective dose” or “effective amount”, as used herein, refers to an amount needed to achieve a desired clinical result or results (e.g. inhibiting receptor-mediated cell activation) based upon trained medical observation and/or quantitative test results. The potency of any administered peptide or compound determines the “effective amount” which can vary for the various compositions that inhibit immune cell activation (i.e., for example, compositions inhibiting TREM ligand-induced myeloid cell activation). Additionally, the “effective amount” of a compound may vary depending on the desired result, for example, the level of myeloid cell activation inhibition desired. The “therapeutically effective amount” necessary for inhibiting differentiation of primary monocytes into immature dendritic cells may differ from the “therapeutically effective amount” necessary for preventing or inhibiting cytokine production.

The term, “agent”, as used herein, refers to any natural or synthetic compound (i.e., for example, a peptide, a peptide variant, or a small molecule).

The term, “composition”, as used herein, refers to any mixture of substances comprising a peptide and/or compound contemplated by the present invention. Such a composition may include the substances individually or in any combination.

The term “therapeutic drug”, as used herein, refers to any pharmacologically active substance capable of being administered which achieves a desired effect. Drugs or compositions can be synthetic or naturally occurring, non-peptide, proteins or peptides, oligonucleotides or nucleotides, polysaccharides or sugars. Drugs or compositions may have any of a variety of activities, which may be stimulatory or inhibitory, such as antibiotic activity, antiviral activity, antifungal activity, steroidal activity, cytotoxic, cytostatic, anti-proliferative, anti-inflammatory, analgesic or anesthetic activity, or can be useful as contrast or other diagnostic agents.

The term “effective dose” as used herein refers to the concentration of any compound or drug contemplated herein that results in a favorable clinical response. In solution, an effective dose may range between approximately 1 ng/ml and 100 mg/ml, preferably between 100 ng/ml and 10 mg/ml, but more preferably between 500 ng/ml and 1 mg/ml.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of the compound or composition of the invention that modulate MIRR including but not limited to the TREM/DAP-12 receptor complex signaling may vary according to factors such as the disease state, age, sex, and weight of the individual, and the ability of the compound or composition to elicit a desired response in the individual. A therapeutically effective amount encompasses an amount in which any toxic or detrimental effects of the compound or composition are outweighed by the therapeutically beneficial effects. In some embodiments, the expression “effective amount” refers to an amount of the compound or composition that is effective for treating cancer and pigmented villonodular synovitis (PVNS).

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “induction therapy” refers to the first treatment given for a disease that is often part of a standard set of treatments. When used by itself, induction therapy is the one accepted as the best treatment. If it doesn't cure the disease or it causes severe side effects, other treatment may be added or used instead. Also called first-line therapy, primary therapy, and primary treatment.

A “maintenance therapy” refers to a medical therapy that is designed to help a primary treatment succeed. For example, maintenance chemotherapy may be given to people who have a cancer in remission in an attempt to prevent a relapse. In other words, treatment that is given to help keep cancer from coming back after it has disappeared following the initial therapy. It may include treatment with drugs, vaccines, or antibodies that kill cancer cells, and it may be given for a long time. This form of treatment is also a common approach for the management of many incurable, chronic diseases such as periodontal disease, Crohn's disease or ulcerative colitis.

The term “in combination with”, as used herein refers to the administration of one or more therapeutic agents that includes simultaneous (concurrent) and consecutive (sequential) administration in any order.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The term “administered” or “administering” a drug or compound, as used herein, refers to any method of providing a drug or compound to a patient such that the drug or compound has its intended effect on the patient. For example, one method of administering is by an indirect mechanism using a medical device such as, but not limited to a catheter, syringe etc. A second exemplary method of administering is by a direct mechanism such as, local tissue administration (i.e., for example, extravascular placement), oral ingestion, transdermal patch, topical, inhalation, suppository etc.)

The term “anti-inflammatory drug” means any compound, composition, or drug useful for preventing or treating inflammatory disease.

The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a drug or compound to a patient during a medical procedure or therapy is contemplated herein as a medical device. A medical device may be “coated” when a medium comprising an anti-inflammatory drug (i.e., for example, the peptides and compositions of the present invention) becomes attached to the surface of the medical device. This attachment may be permanent or temporary. When temporary, the attachment may result in a controlled release of an inflammatory drug.

The term “direct medical implants” include, but are not limited to, urinary and intravascular catheters, dialysis catheters, wound drain tubes, skin sutures, vascular grafts and implantable meshes, intraocular devices, implantable drug delivery systems and heart valves, and the like.

The term “wound care devices” include, but are not limited to, general wound dressings, non-adherent dressings, burn dressings, biological graft materials, tape closures and dressings, surgical drapes, sponges and absorbable hemostats.

The term “surgical devices” include, but are not limited to, surgical instruments, endoscope systems (i.e., catheters, vascular catheters, surgical tools such as scalpels, retractors, and the like) and temporary drug delivery devices such as drug ports, injection needles etc. to administer the medium.

### BRIEF DESCRIPTION OF THE FIGURES

The following figures form part of the present specification and are included to further illustrate aspects of the present invention. The invention may be better understood by reference to the figures in combination with the detailed description of the specific embodiments presented herein.

FIG. 1 presents a schematic representation of one embodiment of the ligand-independent mechanism (the Signaling Chain HOmoOLigomerization, SCHOOL, mechanism) of inhibition of cell surface receptors. In one embodiment, the cell surface receptor comprises TREM-1. In one embodiment, the ligand(s) of TREM-1 is unknown. In one embodiment, the ligand-independent inhibitor of TREM-1 comprises TREM-1 inhibitor peptide sequence GFLSKSLVF (GF9). TREM-1-specific GF9-based SCHOOL inhibitors can advantageously reach their site of action in the cell membrane form both the outside and inside the cell. This allows their use in either free peptide form or formulated in delivery systems for targeted intracellular delivery.

FIG. 2 presents a schematic representation of one embodiment of the ligand-independent mechanism (the Signaling Chain HOmoOLigomerization, SCHOOL, mechanism) of inhibition of cell surface receptors. In one embodiment, the cell surface receptor comprises TREM-2. In one embodiment, the ligand-independent inhibitor of TREM-2 comprises TREM-2 inhibitor peptide sequence IFLIKILAA (IA9). TREM-2-specific IA9-based SCHOOL inhibitors can advantageously reach their site of action in the cell membrane form both the outside and inside the cell. This allows their use in either free peptide form or formulated in delivery systems for targeted intracellular delivery.

FIG. 3 illustrates one embodiment of normal interactions between TREM-2 and a DAP-12 subunit dimer to form a functional TREM-2/DAP-12 receptor complex.

FIG. 4 illustrates one embodiment of interactions between transmembrane domains of TREM-2 and DAP-12 disrupted by using TREM-2 inhibitor peptides of the present invention resulting in “pre-dissociated”, non-functional TREM-2/DAP-12 receptor complex.

FIG. 5 illustrates one embodiment of modulation of binding of the TREM-2 Core and/or Extended peptides of the present invention to the transmembrane domain of the DAP-12 subunit dimer.

FIG. 6 presents various embodiments of TREM-2 peptide inhibitor sequences based upon a general formula, wherein the general formula describes variants of the parent TREM-2 transmembrane sequence.

FIG. 7 presents a schematic representation of one embodiment of the ligand-independent mechanism (the Signaling Chain HOmoOLigomerization, SCHOOL, mechanism) of concurrent inhibition of cell surface receptors. In one embodiment, the cell surface receptors comprise TREM-1 and TREM-2. In one embodiment, the ligand-independent concurrent inhibitor of TREM-1 and TREM-2 comprises a combinatorial peptide sequence GFLSKSLVFIFLIKILAA (GA18) that combines sequences of TREM-1 and TREM-2 inhibitory peptide sequences GFLSKSLVF (GF9) and IFLIKILAA (IA9), respectively. In one embodiment, concurrent inhibitor GA18 can advantageously reach its site of action in the cell membrane form both the outside and inside the cell. This allows its use in either free peptide form or formulated in delivery systems for targeted intracellular delivery.

FIG. 8 presents a schematic representation of one embodiment of trifunctional TREM-2 inhibitor peptide variants and compositions of the present invention. In one embodiment, trifunctional TREM-2 inhibitor peptide variants and compositions of the invention be employed in either free form or incorporated into targeted lipopeptide complexes (LPC), which allows them to reach their site of action from either outside or inside the cell, respectively. In one embodiment targeted LPC are macrophage-targeted LPC. In one embodiment, trifunctional TREM-2 inhibitor peptide comprises an amphipathic peptide IA31. In one embodiment, IA31 is capable of formation of LPC upon interaction with lipids (IA31-LPC). In one embodiment, IA31 comprises two domains, wherein one domain comprises the 9 amino acids-long peptide sequence IA9 to inhibit TREM-2 and another domain comprises 22 amino acid residues-long peptide sequence of the apolipoprotein A-I helix 6 (PA22). In one embodiment, domain PA22 contains a sulfoxidized methionine residue to provide targeted delivery to cells (e.g., macrophages) via interaction with scavenger receptor (e.g., type A scavenger receptor, SR-A, expressed on macrophages) and a binding site for class B type I scavenger receptor (SR-BI) to provide hepatic clearance of IA31-LPC via interaction with SR-BI expressed on hepatocytes and/or targeted delivery to cancer cells via interaction with SR-BI expressed on cancer cells.

FIG. 9 presents the exemplary data of one embodiment showing that: 1) lipid and peptide components of IA31-LPC are both delivered to macrophages intracellularly and 2) sulfoxidation of the methionine residue of IA31 results in the significantly increased uptake (endocytosis) of IA31-LPC by macrophages. As described herein, IA31-LPC that contain rhodamine B (Rho B)-labeled lipid and DyLight 405-labeled IA31 (the latter with oxidized or unoxidized methionine residue) were incubated with macrophages for 4 hours. Then, cells were lysed and fluorescence intensities of Rho B and DyLight 405 were measured and normalized to the protein content. All results are expressed as the mean±SEM (n=4). ***, p<0.001 as compared with medium-treated macrophages.

FIG. 10 presents the exemplary data of one embodiment showing significant suppression of inflammatory response by human peripheral blood mononuclear cell (PBMC) stimulated with lipopolysaccharide (LPS) and treated with either free peptides GF9, IA9 and GA18 or with GA31-LPC and IA31-LPC. In one embodiment, GF9 and IA9 are formulated in a mixture of propylene glycol, ethanol and Tween-80 (GF9-P and IA9-P, respectively). In one embodiment, GF9 and GA18 are formulated in a pharmacologically acceptable excipient, sulfobutylether-beta-cyclodextrin (SBECD). In one embodiment, SBECD represents Dexolve, a generic form of Captisol (GF9-D and GA18-D). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment. GA31-LPC comprises a complex of GA31 with one lipid component. In one embodiment, this lipid component comprises POPC (GA31-LPC1). In one embodiment, GA31-LPC1 comprises GA31-LPC1 with decreased POPC-GA31 molar ration (GA31-LPC1-20). In one embodiment, IA31-LPC comprises a complex of IA31 with one lipid component. In one embodiment, this lipid component comprises POPC (IA31-LPC1). As described herein, cells were incubated in a 96-well plate at 37° C. with 5% C02 for one hour. Then, vehicle, dexamethasone (DEX, positive control) and TREM-1 and TREM-2 inhibitory formulations of GF9, IA9, GA18, GA31-LPC and IA31-LPC1 were added to the appropriate wells at the indicated final concentrations. After 1 hour, 10 uL of LPS were added. Cells were incubated at 37° C. with 5% C02 for 24 hours. 4 hours before 24-hour harvest 20 uL of Alamar Blue was added to all wells. Plates were incubated at 37° C. with 5% C02 until the 24-hour time point. At 24 hours post stimulation, plates were read for assessment of cell viability and cell culture supernatants were collected, centrifuge to remove cells and analyzed for IL-1b, IL-6, and TNFa. All results are expressed as the mean SEM (n=4). *, p<0.05; **, p<0.01 vs vehicle-treated cells.

FIG. 11 presents the exemplary data of one embodiment showing significant suppression of systemic inflammatory response in mice treated with either free peptides GF9, IA9 and GA18 or with GA31-LPC and IA31-LPC at the indicated doses 1 h before or 1 hour after lipopolysaccharide (LPS) challenge. In one embodiment, GF9 and IA9 are formulated in a mixture of propylene glycol, ethanol and Tween-80 (GF9-P and IA9-P, respectively). In one embodiment, GF9 and GA18 are formulated in a pharmacologically acceptable excipient, sulfobutylether-beta-cyclodextrin (SBECD). In one embodiment, SBECD represents Dexolve, a generic form of Captisol (GF9-D and GA18-D). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment. GA31-LPC comprises a complex of GA31 with one lipid component. In one embodiment, this lipid component comprises POPC (GA31-LPC1). In one embodiment, GA31-LPC1 comprises GA31-LPC1 with decreased POPC-GA31 molar ration (GA31-LPC1-20). In one embodiment, IA31-LPC comprises a complex of IA31 with one lipid component. In one embodiment, this lipid component comprises POPC (IA31-LPC1). Proinflammatory cytokines were analyzed in blood samples taken at 4 h post LPS challenge. All results are expressed as the mean±standard error of the mean (SEM) (n=3). *, p<0.05; **, p<0.01 vs vehicle-treated mice. In one embodiment, the route of administration is intraperitoneal (i.p.). In one embodiment, the route of administration is intravenous (i.v., not shown). In one embodiment, no differences were observed between i.p. and i.p. routes of administration.

FIG. 12 presents the exemplary data of one embodiment showing significant survival extension of mice treated with free peptide GF9 or GA31-LPC at the indicated doses 1 h before or 1 or 3 hours after lipopolysaccharide (LPS) challenge. In one embodiment, dexamethasone was used as a positive control. In one embodiment, GF9 is formulated in a pharmacologically acceptable excipient, sulfobutylether-beta-cyclodextrin (SBECD). In one embodiment, SBECD represents Dexolve, a generic form of Captisol (GF9-D). In one embodiment. GA31-LPC comprises a complex of GA31 with one lipid component. In one embodiment, this lipid component comprises POPC (GA31-LPC1). In one embodiment, a route of administration is intraperitoneal (i.p.). The Kaplan-Meier method was used for survival analysis. *, p<0.05; **, p<0.01; ***, p<0.001, all vs vehicle-treated mice.

FIG. 13 presents the exemplary data of one embodiment showing significant survival extension of mice treated with free peptides IA9 and GA18 or with IA31-LPC at the indicated doses 1 h before or 1 or 3 hours after lipopolysaccharide (LPS) challenge. In one embodiment, IA9 is formulated in a mixture of propylene glycol, ethanol and Tween-80 (IA9-P). In one embodiment, GA18 is formulated in a pharmacologically acceptable excipient, sulfobutylether-beta-cyclodextrin (SBECD). In one embodiment, SBECD represents Dexolve, a generic form of Captisol (GA18-D). In one embodiment, IA31-LPC comprises a complex of IA31 with one lipid component. In one embodiment, this lipid component comprises POPC (IA31-LPC1). In one embodiment, dexamethasone was used as a positive control. In one embodiment, this lipid component comprises POPC (IA31-LPC1). In one embodiment, a route of administration is intraperitoneal (i.p.). The Kaplan-Meier method was used for survival analysis. *, p<0.05; **, p<0.01; ***, p<0.001, all vs vehicle-treated mice.

FIG. 14 presents the exemplary data of one embodiment showing significant survival extension of mice treated with free peptide GF9 or GA31-LPC at the indicated doses 1 h before or 6 or 12 hours after cecal slurry (CS) challenge. In one embodiment, combination antibiotic ceftriaxone and metronidazole administered intraperitoneally (i.p.) was used as a positive control. In one embodiment, GF9 is formulated in a pharmacologically acceptable excipient, sulfobutylether-beta-cyclodextrin (SBECD). In one embodiment, SBECD represents Dexolve, a generic form of Captisol (GF9-D). In one embodiment. GA31-LPC comprises a complex of GA31 with one lipid component. In one embodiment, this lipid component comprises POPC (GA31-LPC1). In one embodiment, a route of administration is i.p. The Kaplan-Meier method was used for survival analysis. *, p<0.05; **, p<0.01; ***, p<0.001, all vs vehicle-treated mice.

FIG. 15 presents the exemplary data of one embodiment showing significant survival extension of mice treated with free peptides IA9 and GA18 or with IA31-LPC at the indicated doses 1 h before or 6 or 12 hours after cecal slurry (CS) challenge. In one embodiment, IA9 is formulated in a mixture of propylene glycol, ethanol and Tween-80 (IA9-P). In one embodiment, GA18 is formulated in a pharmacologically acceptable excipient, sulfobutylether-beta-cyclodextrin (SBECD). In one embodiment, SBECD represents Dexolve, a generic form of Captisol (GA18-D). In one embodiment, IA31-LPC comprises a complex of IA31 with one lipid component. In one embodiment, this lipid component comprises POPC (IA31-LPC1). In one embodiment, combination antibiotic ceftriaxone and metronidazole administered intraperitoneally (i.p.) was used as a positive control. In one embodiment, this lipid component comprises POPC (IA31-LPC1). In one embodiment, a route of administration is i.p. The Kaplan-Meier method was used for survival analysis. *, p<0.05; **, p<0.01; ***, p<0.001, all vs vehicle-treated mice.

FIG. 16 presents a schematic representation of one embodiment of the similarities of the pathogenesis of lung injuries induced by ionizing radiation, chemicals, bacteria or viruses. In one embodiment, chemical is sulfur mustard (SM). In one embodiment, bacterium is Bordetella pertussis. In one embodiment, virus is severe acute respiratory syndrome coronavirus 2 (SARS-CoV-2). While not being bound to any particular theory, it is believed that TREM-1 and TREM-2 mediate release of proinflammatory cytokines, drive pathological lung inflammation and correlate with poor clinical outcome. In one embodiment, proinflammatory cytokines are tumor necrosis factor alpha (TNFa), interleukin (IL)-6, IL-1b and monocyte chemoattractant protein 1 (MCP-1).

FIG. 17 presents the exemplary data of one embodiment showing that TREM-1 does not contribute to bacterial control in Bordetella pertussis (B. pertussis) infection. Bacterial burden was assessed at 4 and 7 dpi by plating the lungs of B. pertussis-challenged C57BL/6 mice receiving free peptide GF9 or GA31-LPC at the indicated doses on Bordet-Gengou agar plates.

In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment, each data point represents the average of three technical replicates obtained from one of four individual mice (n=4). Data are shown as means with standard deviations. In one embodiment, p values are determined by two-way ANOVA using Sidak's multiple-comparison test. In one embodiment, the test shows the lack of significant differences between groups.

FIG. 18 presents the exemplary data of one embodiment showing significant suppression of lung inflammatory response in mice infected by Bordetella pertussis (B. pertussis) and treated intraperitoneally with free peptide GF9 or GA31-LPC at the indicated doses starting Day 0. In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. As described herein, bacterial inocula prepared in sterile phosphate-buffered saline (PBS) following 48 hours incubation on Bordet-Gengou (BG) agar were used to infect mice intranasally (2×10\hat{ }6 colony-forming units, CFU, B. pertussis). At 4 days post infection (dpi), lung tissues were harvested and RNA was quantified. The hypoxanthine phosphoribosyltransferase (HPRT) gene was used as an internal housekeeping control gene, with all genes normalized to the HPRT gene. Cytokine and chemokine RNA expression was calculated as fold change compared with vehicle-inoculated control animals. In one embodiment, cytokines and chemokines are CCL2 (or monocyte chemoattractant protein-1, MCP-1), CXCL3 (C-X-C motif chemokine ligand 3) and TNFa. All results are expressed as the mean±SEM (n=4). *, p<0.05 vs vehicle-treated mice.

FIG. 19 presents the exemplary data of one embodiment showing significant suppression of inflammatory lung pathology in mice infected by Bordetella pertussis (B. pertussis) and treated intraperitoneally with free peptide GF9 or GA31-LPC at the indicated doses. In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment, the treatment started Day 0 (prevention). In one embodiment, the treatment started Day 3 (treatment). As described herein, bacterial inocula prepared in sterile phosphate-buffered saline (PBS) following 48 hours incubation on Bordet-Gengou (BG) agar were used to infect mice intranasally (2×10\hat{ }6 colony-forming units, CFU, B. pertussis). At 4 days post infection (dpi), lungs were perfused with PBS before removal into 10% (wt/vol) neutral buffered formalin. Hematoxylin-eosin staining was performed. A semi-quantitative scoring system based on the degree of infiltrate in the bronchovascular region and the degree of tissue consolidation observed was used.

FIG. 20 presents the exemplary data of one embodiment showing good tolerability of free peptide GF9 and GA31-LPC in sulfur mustard (SM)-challenged rats. In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment, 25 mg/kg GF9 and 13 mg/kg GA31-LPC were administered intraperitoneally 0.5 and 4 hrs post-challenge, and then every 24 hrs for 7 days starting Day 2. In one embodiment, SM exposures were conducted at a nominal concentration of 150 mg SM/m3 with varying durations to reach a targeted inhaled dose of 0.8 mg/kg. All results are expressed as the mean±standard deviation (n=13).

FIG. 21 presents the exemplary data of one embodiment showing increase of peripheral oxygen saturation at 1 and 7 days post-challenge in sulfur mustard (SM)-challenged rats treated with GF9 and GA31-LPC. In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment, 25 mg/kg GF9 and 13 mg/kg GA31-LPC were administered intraperitoneally 0.5 and 4 hrs post-challenge, and then every 24 hrs for 7 days starting Day 2. In one embodiment, SM exposures were conducted at a nominal concentration of 150 mg SM/m3 with varying durations to reach a targeted inhaled dose of 0.8 mg/kg. All results are expressed as the mean±standard deviation (n=13).

FIG. 22 presents the exemplary data of one embodiment showing significant survival extension of sulfur mustard (SM)-challenged rats treated with free peptide GF9 and GA31-LPC. In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment, 25 mg/kg GF9 and 13 mg/kg GA31-LPC were administered intraperitoneally 0.5 and 4 hrs post-challenge, and every 24 hrs for 7 days starting Day 2. In one embodiment, 31%, 54% and 77% survival were observed in vehicle-, GF9-, and GA31-LPC-treated groups, respectively. In one embodiment, vehicle is phosphate-buffered saline (PBS), pH 7.4. In one embodiment, survival at 28 days continued to be highest in GA31-LPC-treated group (54%) compared to vehicle—(31%) and GF9-treated (38%) groups. In one embodiment, SM exposures were conducted at a nominal concentration of 150 mg SM/m3 with varying durations to reach a targeted inhaled dose of 0.8 mg/kg. In one embodiment, a route of administration is intraperitoneal (i.p.). The Kaplan-Meier method was used for survival analysis. *, p<0.05 vs vehicle-treated rats.

FIG. 23 presents the exemplary data of one embodiment showing significant attenuation of lung inflammation in mice challenged with intratracheal bleomycin and treated daily intraperitoneally with free peptides GF9, IA9 and GA-18 or GA31-LPC and IA31-LPC at the indicated doses. In one embodiment, GF9 and IA9 are formulated in a mixture of propylene glycol, ethanol and Tween-80 (GF9-P and IA9-P, respectively). In one embodiment, GF9 and GA18 are formulated in a pharmacologically acceptable excipient, sulfobutylether-beta-cyclodextrin (SBECD). In one embodiment, SBECD represents Dexolve, a generic form of Captisol (GF9-D and GA18-D). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment. GA31-LPC comprises a complex of GA31 with one lipid component. In one embodiment, this lipid component comprises POPC (GA31-LPC1). In one embodiment, GA31-LPC1 comprises GA31-LPC1 with decreased POPC-GA31 molar ration (GA31-LPC1-20). In one embodiment, IA31-LPC comprises a complex of IA31 with one lipid component. In one embodiment, this lipid component comprises POPC (IA31-LPC1). In one embodiment, the treatment started Day 1 (prevention). In one embodiment, the treatment started Day 15 (treatment). In one embodiment, total cells and total protein of bronchoalveolar lavage fluid (BALF) were analyzed at Day 7. In one embodiment, total cells and total protein of bronchoalveolar lavage fluid (BALF) were analyzed at Day 21. In one embodiment, total cells and total protein of bronchoalveolar lavage fluid (BALF) were analyzed at Day 28. All results are expressed as the mean±SEM (n=8). **, p<0.01 vs vehicle-treated mice challenged with inhaled bleomycin.

FIG. 24 presents the exemplary data of one embodiment showing significant attenuation of lung damage and fibrosis in mice challenged with intratracheal bleomycin and treated daily intraperitoneally with free peptides GF9, IA9 and GA-18 or GA31-LPC and IA31-LPC at the indicated doses. In one embodiment, GF9 and IA9 are formulated in a mixture of propylene glycol, ethanol and Tween-80 (GF9-P and IA9-P, respectively). In one embodiment, GF9 and GA18 are formulated in a pharmacologically acceptable excipient, sulfobutylether-beta-cyclodextrin (SBECD). In one embodiment, SBECD represents Dexolve, a generic form of Captisol (GF9-D and GA18-D). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. In one embodiment. GA31-LPC comprises a complex of GA31 with one lipid component. In one embodiment, this lipid component comprises POPC (GA31-LPC1). In one embodiment, GA31-LPC1 comprises GA31-LPC1 with decreased POPC-GA31 molar ration (GA31-LPC1-20). In one embodiment, IA31-LPC comprises a complex of IA31 with one lipid component. In one embodiment, this lipid component comprises POPC (IA31-LPC1). In one embodiment, the treatment started Day 1 (prevention). In one embodiment, the treatment started Day 15 (treatment). In one embodiment, the severity of lung damage and fibrosis was analyzed at Day 7. In one embodiment, the severity of lung damage and fibrosis was analyzed at Day 21. In one embodiment, the severity of lung damage and fibrosis was analyzed at Day 28. All results are expressed as the mean±SEM (n=8). *, p<0.05 vs vehicle-treated mice challenged with inhaled bleomycin.

FIG. 25 presents the exemplary data of one embodiment showing significant suppression of arthritis in mice with collagen-induced arthritis (CIA) treated daily intraperitoneally with free peptides GF9 and IA9 or GA31-LPC and IA31-LPC at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment. GA31-LPC comprises a complex of GA31 with one lipid component. In one embodiment, this lipid component comprises POPC (GA31-LPC1). In one embodiment, IA31-LPC comprises a complex of IA31 with one lipid component. In one embodiment, this lipid component comprises POPC (IA31-LPC1). In one embodiment, the treatment started Therapy Day 1 (Study Day 28). In one embodiment, macroscopic signs of arthritis are scored as follows: a) each paw receives a score; b) 0=no visible effects of arthritis; c) 1=edema and/or erythema of 1 digit; d) 2=edema and/or erythema of 2 digits; e) 3=edema and/or erythema of more than 2 digits; f) 4=severe arthritis of entire paw and digits; g) the score for each paw of each animal is recorded; h) the arthritic index (AI) is the sum of the individual paw scores for each animal; i) the AI for each animal is recorded; j) the maximum AI for one animal is 16. All results are expressed as the mean±SEM (n=12). **, p<0.01; ***, p<0.001 vs vehicle-treated mice with CIA.

FIG. 26 presents the exemplary data of one embodiment showing significant suppression of arthritis in mice with collagen-induced arthritis (CIA) treated daily intraperitoneally with free peptides GF9, IA9 and GA18 at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment, GF9 and GA18 are formulated in a pharmacologically acceptable excipient, sulfobutylether-beta-cyclodextrin (SBECD). In one embodiment, SBECD represents Dexolve, a generic form of Captisol (GF9-D and GA18-D). In one embodiment, GF9-P and IA9-P are concurrently administered at the indicated doses (1:1 by dose). In one embodiment, the treatment started Therapy Day 1 (Study Day 28). In one embodiment, macroscopic signs of arthritis are scored as follows: a) each paw receives a score; b) 0=no visible effects of arthritis; c) 1=edema and/or erythema of 1 digit; d) 2=edema and/or erythema of 2 digits; e) 3=edema and/or erythema of more than 2 digits; f) 4=severe arthritis of entire paw and digits; g) the score for each paw of each animal is recorded; h) the arthritic index (AI) is the sum of the individual paw scores for each animal; i) the AI for each animal is recorded; j) the maximum AI for one animal is 16. In one embodiment, treatment with GA18-D significantly reduces arthritis compared with concurrent treatment with GF9-P and IA9-P (1:1 by dose). All results are expressed as the mean±SEM (n=12). **, p<0.01; ***, p<0.001 vs vehicle-treated mice with CIA. #, p<0.05 vs mice with CIA treated concurrently with GF9-P and IA9-P (1:1).

FIG. 27 presents the exemplary data of one embodiment showing good tolerability of GF9, IA9, GA31-LPC and IA31-LPC in mice with collagen-induced arthritis (CIA) treated daily intraperitoneally with free peptides GF9 and IA9 or GA31-LPC and IA31-LPC at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment. GA31-LPC comprises a complex of GA31 with one lipid component. In one embodiment, this lipid component comprises POPC (GA31-LPC1). In one embodiment, IA31-LPC comprises a complex of IA31 with one lipid component. In one embodiment, this lipid component comprises POPC (IA31-LPC1). In one embodiment, mouse body weight is measured every other day from Therapy Day 1 (study Day 28) to Therapy Day 14. In one embodiment, mean body weight changes are calculated as a percentage of the difference between beginning (Therapy Day 1) and final (Therapy Day 14) body weights. All results are expressed as mean±SEM (n=12).

FIG. 28 presents the exemplary data of one embodiment showing significant suppression of systemic inflammation in mice with collagen-induced arthritis (CIA) treated daily intraperitoneally with free peptides GF9 and IA9 or GA31-LPC and IA31-LPC at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment. GA31-LPC comprises a complex of GA31 with one lipid component. In one embodiment, this lipid component comprises POPC (GA31-LPC1). In one embodiment, IA31-LPC comprises a complex of IA31 with one lipid component. In one embodiment, this lipid component comprises POPC (IA31-LPC1). In one embodiment, the treatment started Therapy Day 1 (Study Day 28). In one embodiment, terminal plasma levels of proinflammatory cytokines are analyzed at Therapy Day 14. In one embodiment, proinflammatory cytokines are IL-1b, IL-6 and CSF-1 (macrophage-colony stimulating factor, MCSF or CSF-1). All results are expressed as the mean±SEM (n=12). *, p<0.05; **, p<0.01; ***, p<0.001; ****, p<0.0001 vs vehicle-treated mice with CIA.

FIG. 29 presents the exemplary data of one embodiment showing significant suppression of local inflammation in mice with collagen-induced arthritis (CIA) treated daily intraperitoneally with free peptides GF9 and IA9 or GA31-LPC and IA31-LPC at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment. GA31-LPC comprises a complex of GA31 with one lipid component. In one embodiment, this lipid component comprises POPC (GA31-LPC1). In one embodiment, IA31-LPC comprises a complex of IA31 with one lipid component. In one embodiment, this lipid component comprises POPC (IA31-LPC1). In one embodiment, the treatment started Therapy Day 1 (Study Day 28). In one embodiment, levels of proinflammatory cytokines in mouse joints are analyzed at Therapy Day 14. In one embodiment, joints are mouse knees. In one embodiment, proinflammatory cytokines are TNFa, IL-1b, IL-6 and CSF-1 (also referred to as macrophage-colony stimulating factor, MCSF). All results are expressed as the mean±SEM (n=12). *, p<0.05; **, p<0.01; ***, p<0.001; ****, p<0.0001 vs vehicle-treated mice with CIA.

FIG. 30 presents the exemplary data of one embodiment showing significant suppression of joint inflammation and damage in mice with collagen-induced arthritis (CIA) treated daily intraperitoneally with free peptides GF9 and IA9 or GA31-LPC and IA31-LPC at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment. GA31-LPC comprises a complex of GA31 with one lipid component. In one embodiment, this lipid component comprises POPC (GA31-LPC1). In one embodiment, IA31-LPC comprises a complex of IA31 with one lipid component. In one embodiment, this lipid component comprises POPC (IA31-LPC1). In one embodiment, the treatment started Therapy Day 1 (Study Day 28). In one embodiment, joint inflammation and damage are analyzed at Therapy Day 14. In one embodiment, six joints from each animal are processed for histopathological evaluation. In one embodiment, the joints are assessed using 0-5 scale for inflammation, pannus formation, cartilage damage, bone resorption and periosteal new bone formation (Panel A). In one embodiment, a summed histopathology score (sum of five parameters, 0-25 scale) is also determined (Panel B). All results are expressed as the mean SEM (n=12). ****, p<0.0001 vs vehicle-treated mice with CIA.

FIG. 31 presents the exemplary data of one embodiment showing significant reduction of cartilage destruction and immune cell infiltration in mice with collagen-induced arthritis (CIA) treated daily intraperitoneally (i.p.) with free peptides GF9 and IA9 or GA31-LPC and IA31-LPC at the indicated doses. In one embodiment, prednisolone is used as positive control. In one embodiment, GF9 and IA9 are formulated in a mixture of propylene glycol, ethanol and Tween-80 (GF9-P and IA9-P, respectively). In one embodiment. GA31-LPC comprises a complex of GA31 with one lipid component. In one embodiment, this lipid component comprises POPC (GA31-LPC1). In one embodiment, IA31-LPC comprises a complex of IA31 with one lipid component. In one embodiment, this lipid component comprises POPC (IA31-LPC1). In one embodiment, the treatment started Therapy Day 1 (Study Day 28). In one embodiment, cartilage destruction is analyzed and scored at Therapy Day 14 by using joint sections stained for type IV collagen (Panel A). Exemplary images of the joint sections stained for type IV collagen are presented in Panel B. In one embodiment, synovial lining of joints of mice with CIA treated daily i.p. with free peptides GF9 and IA9 or GA31-LPC and IA31-LPC at the indicated doses is stained for immune cells and number of the cells is counted (Panel C). In one embodiment, immune cells are CD68-, F4/80-, TREM-2- and TREM-1-positive cells (Panel C). All results are expressed as the mean±SEM (n=12). ****, p<0.0001 vs vehicle-treated mice with CIA.

FIG. 32 presents the exemplary data of one embodiment showing significant tumor growth inhibition and tumor shrinkage and significantly increased number of tumor-free survivors (TFS) and complete regression (CR) in human pancreatic tumor PANC-1 xenograft-carrying nude mice treated intraperitoneally (i.p.) with 25 mg/kg free peptide GF9 (but not 13 mg/kg GA31-LPC) in combination with chemo (80 mg/kg Gemcitabine i.p. Q3Dx4 and 30 mg/kg Abraxane intravenously, i.v., Q3Dx4) starting Day 1 and then continuing as post-chemo maintenance therapy (Panel A). In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. As described herein, female athymic nude mice (Crl:NU(NCr)-Foxn1^{nu}) inoculated subcutaneously in the right flank with 0.1 mL of a 50% DMEM/50% Phenol Red-free Matrigel mixture containing a suspension of 5×10\hat{ }6 cells/mouse of PANC-1 tumor cells. Tumor volumes were calculated utilizing the formula: Tumor volume (mm³)=(a×b\hat{ }2/2) where ‘b’ is the smallest diameter and ‘a’ is the largest diameter. Twenty-six days following inoculation, mice with tumor volumes of 71-159 mm\hat{ }3 were randomized into groups of ten mice, each with a group mean tumor volume of 107-108 mm\hat{ }3 by random equilibration. Tumor volumes and body weights were recorded when the mice were randomized and two times weekly thereafter. The relative tumor volume (RTV) was calculated using the following formula: RTV=(tumor volume on measured day)/(tumor volume on day 0). Dosing was performed as described herein. Gross observations were recorded daily. In one embodiment, individual mice responses were also identified as either TFS, CR, or partial regression (PR) throughout the study (Panel B) based on the criteria: CR=tumor volume of less than 13.5 mm\hat{ }3 for three consecutive measurements; TFS=any animal with a CR response at the end of the study was additionally classified as a tumor-free survivor; PR=50% or less of Day 1 tumor volume for three consecutive measurements with at least one of the measurements equal to or greater than 13.5 mm\hat{ }3. All results are expressed as the mean±SEM (n=10). *, p<0.05 vs mice treated with chemo alone.

FIG. 33 presents the exemplary data of one embodiment showing significant tumor growth inhibition and tumor shrinkage and significantly increased number of tumor-free survivors (TFS) and complete regression (CR) in human pancreatic tumor PANC-1 xenograft-carrying nude mice treated intraperitoneally (i.p.) with 13 mg/kg GA31-LPC (but not 25 mg/kg free peptide GF9) starting Day 13 as post-chemo maintenance therapy (chemo: 80 mg/kg Gemcitabine i.p. Q3Dx4 and 30 mg/kg Abraxane intravenously, i.v., Q3Dx4) (Panel A). In one embodiment, GF9 is formulated in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. As described herein, female athymic nude mice (Crl:NU(NCr)-Foxn1^{nu}) inoculated subcutaneously in the right flank with 0.1 mL of a 50% DMEM/50% Phenol Red-free Matrigel mixture containing a suspension of 5×10\hat{ }6 cells/mouse of PANC-1 tumor cells. Tumor volumes were calculated utilizing the formula: Tumor volume (mm³)=(a×b\hat{ }2/2) where ‘b’ is the smallest diameter and ‘a’ is the largest diameter. Twenty-six days following inoculation, mice with tumor volumes of 71-159 mm\hat{ }3 were randomized into groups of ten mice, each with a group mean tumor volume of 107-108 mm\hat{ }3 by random equilibration. Tumor volumes and body weights were recorded when the mice were randomized and two times weekly thereafter. The relative tumor volume (RTV) was calculated using the following formula: RTV=(tumor volume on measured day)/(tumor volume on day 0). Dosing was performed as described herein. Gross observations were recorded daily. In one embodiment, individual mice responses were also identified as either TFS, CR, or partial regression (PR) throughout the study (Panel B) based on the criteria: CR=tumor volume of less than 13.5 mm\hat{ }3 for three consecutive measurements; TFS=any animal with a CR response at the end of the study was additionally classified as a tumor-free survivor; PR=50% or less of Day 1 tumor volume for three consecutive measurements with at least one of the measurements equal to or greater than 13.5 mm\hat{ }3. All results are expressed as the mean±SEM (n=10). ***, p<0.001 vs mice treated with chemotherapy alone.

### DETAILED DESCRIPTION OF THE INVENTION

The present invention is related to the field of pulmonary therapeutics. In particular, the compositions described herein are used in methods of treating lung disease and injury including, but not limited, to lung injuries caused by ionizing radiation, chemicals, bacteria and viruses, acute lung injury (ALI), acute respiratory distress syndrome (ARDS), chronic lung injury, COVID infection, sepsis and related conditions. These compositions include, but are not limited to, peptide variants and compositions that inhibit activity of receptor complexes formed by triggering receptors expressed on myeloid cells (TREM; i.e., TREM-1, TREM-2, TREM-3 or TREM-4) and DNAX activation protein of 12 kDa (DAP12).

In particular, the compositions described herein are used in methods of treating inflammation-associated diseases and conditions. In one embodiment, said inflammation-associated diseases and conditions are selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, allergic diseases, acute radiation syndrome (ARS), empyema, acute mesenteric ischemia, hemorrhagic shock, multiple sclerosis, autoimmune diseases (e.g., rheumatoid arthritis, psoriatic arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory lung diseases (e.g., interstitial pneumonitis and asthma), retinopathy (e.g., retinopathy of prematurity and diabetic retinopathy), neurodegenrative diseases (e.g., Alzheimer's, Parkinson's and Huntington's diseases), gastroenterological diseases and conditions (e.g. inflammatory bowel disease, Crohn's disease, celiac disease), Guillain-Barre syndrome, Hashimoto's disease, pernicious anemia, primary biliary cirrhosis, chronic active hepatitis, alcohol-induced liver disease, nonalcoholic fatty liver disease and non-alcoholic steatohepatitis, skin problems (e.g. atopic dermatitis, psoriasis, pemphigus vulgaris), cardiovascular problems (e.g. autoimmune pericarditis), allergic diathesis (e.g. delayed type hypersensitivity), contact dermatitis, herpes simplex/zoster, respiratory conditions (e.g. allergic alveolitis), inflammatory conditions (e.g. myositis), ankylosing spondylitis, tissue/organ transplant (e.g., heart/lung transplants) rejection reactions, brain and spinal cord injuries, and other diseases and conditions where inflammation is involved.

In one embodiment, the peptide variants and compositions of this disclosure include, but are not limited to, peptide variants and compositions that inhibit activity of a receptor complex formed by a TREM receptor (including but not limited to TREM-1 and TREM-2) and DAP12. These compositions further include, but are not limited to, combinatorial peptide variants and compositions that inhibit activity of two or more receptors expressed on the same or different cells involved in the pathogenesis of ARDS, COVID infection, cytokine storms, sepsis and related conditions and other inflammation-associated diseases and conditions. In one embodiment, said inflammation-associated disease is rheumatoid arthritis (RA) where the combinatorial peptide variants of the invention can therapeutically target TCR and a particular TREM receptor simultaneously (all beneficial for the treatment of RA). In one embodiment, said combinatorial peptide variants of the invention can therapeutically target TCR and different TREM receptors (e.g., TREM-1 and TREM-2) simultaneously (all beneficial for the treatment of RA). Examples include, but are not limited to, the combinatorial peptide GFLSKSLVFIFLIKILAA (GA-18) (SEQ. ID NO: 49) that is designed to inhibit TREM-1 and TREM-2 simultaneously, the combinatorial peptide MWKTPTLKYFGFLSKSLVF (MF-19) (SEQ. ID NO: 56) that is designed to inhibit TCR and TREM-1 simultaneously, the combinatorial peptide MWKTPTLKYFIFLIKILAA (MA-19) (SEQ. ID NO: 61) that is designed to inhibit TCR and TREM-2 simultaneously, and the combinatorial peptide MWKTPTLKYFGFLSKSLVFIFLIKILAA (MA-28) (SEQ. ID NO: 63) that is designed to inhibit TCR, TREM-1 and TREM-2 simultaneously.

In one embodiment, the invention as disclosed herein provides for methods of treating lung disease and injury including, but not limited to, ARDS and related conditions using inhibitors of the TREM-1 and/or TREM-2 pathway or the combinatorial peptide variants of the invention. In one embodiment, the method further provides for treating other inflammation-associated diseases and conditions including, but not limited to, cancer, retinopathy, autoimmune, cardiovascular, sepsis, and related conditions using inhibitors of the TREM-1 and/or TREM-2 pathway or the combinatorial peptide variants of the invention. These inhibitors include. but are not limited to, peptide variants and compositions that modulate the TREM-1- and/or TREM-2-mediated immunological responses beneficial for the treatment of lung disease and injury and other inflammatory diseases and disorders. In one embodiment, the present invention also provides methods for predicting the efficacy of TREM-1- and/or TREM-2-targeted therapies in lung disease and conditions by analyzing biological samples for the presence of myeloid cells and for the TREM-1 and/or TREM-2 expression levels.

As described herein, it is surprisingly found that the presently disclosed peptide variants and compositions are capable of inhibiting the TREM-1 and/or TREM-2 signaling pathways and can be synthesized and used for targeted treatment of lung disease, lung injury and related conditions. In one embodiment, combinatorial TREM-1 and TREM-2 inhibitor peptide variants and compositions, as well as trifunctional TREM-1 and/or TREM-2 inhibitory peptide variants and compositions are demonstrated to solve numerous problems which otherwise are associated with high dosages of therapeutic agents (TAs) and imaging probes required and the lack of control and reproducibility of formulations, especially in large-scale production.

In one embodiment, the present invention relates to a targeted treatment, prevention and/or detection of lung disease and injury including, but not limiting to, ARDS, including occurrences of ARDS caused by upper respiratory tract infections such as SARS (including but not limited to SARS-CoV-1 and SARS-CoV-2) and MERS, acute lung injury, pulmonary fibrosis (idiopathic), bleomycin-induced pulmonary fibrosis, mechanical ventilator induced lung injury, COPD, chronic bronchitis, emphysema, bronchiolitis obliterans after lung transplantation and lung transplantation-induced acute graft dysfunction, including treatment, prevention or prevention of progression of primary graft failure, ischemia-reperfusion injury, reperfusion injury, reperfusion edema, allograft dysfunction, pulmonary reimplantation response, bronchiolitis obliterans after lung transplantation and/or primary graft dysfunction (PGD) after organ transplantation, in particular in lung transplantation.

These compositions further include, but are not limited to, combinatorial inhibitor peptide variants and compositions designed by using a signaling chain homooligomerization (SCHOOL) model (Sigalov 2010b, Sigalov 2010a) to target two or more MIRR receptors concurrently. Although it is not necessary to understand the mechanism of an invention, it is believed that the use of these combinatorial peptides and compositions is advantageous as compared to the use of combinations of separate peptides and compositions that target single cell receptor. In one embodiment, combinatorial peptide variants and compositions are designed to target TREM-1 and TREM-2 concurrently. In certain embodiments, the peptide variants and compositions of the invention are formulated in lipopeptide complexes (LPC) for their targeted delivery.

### I. TREM-1, TREM-2 and Combinatorial Inhibitor Peptide Improvements Over Conventional Therapeutic Peptides

It has been reported that the main limitations generally attributed to therapeutic peptides are: a short half-life because of their rapid degradation by proteolytic enzymes of the digestive system and blood plasma; rapid removal from the circulation by the liver (hepatic clearance) and kidneys (renal clearance); poor ability to cross physiological barriers because of their general hydrophilicity; high conformational flexibility, resulting sometimes in a lack of selectivity involving interactions with different receptors/targets (poor specific biodistribution), causing activation of several targets and leading to side effects; eventual risk of immunogenic effects; and high synthetic and production costs (the production cost of a 5000 Da molecular mass peptide exceeds the production cost of a 500 Da molecular mass small molecule by more than 10-fold but clearly not 100-fold) (Vlieghe et al. 2010).

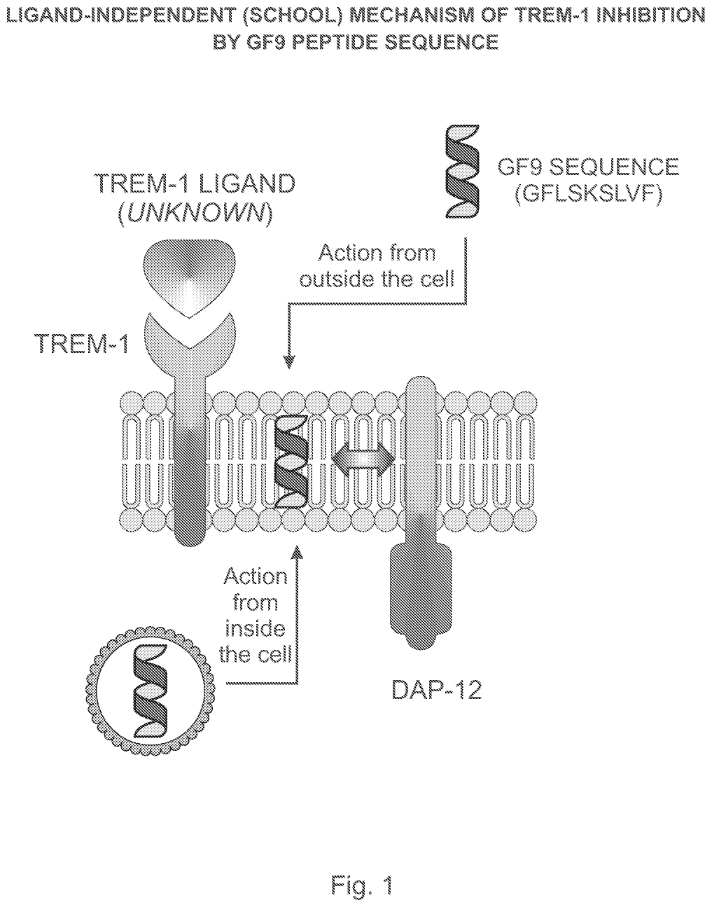

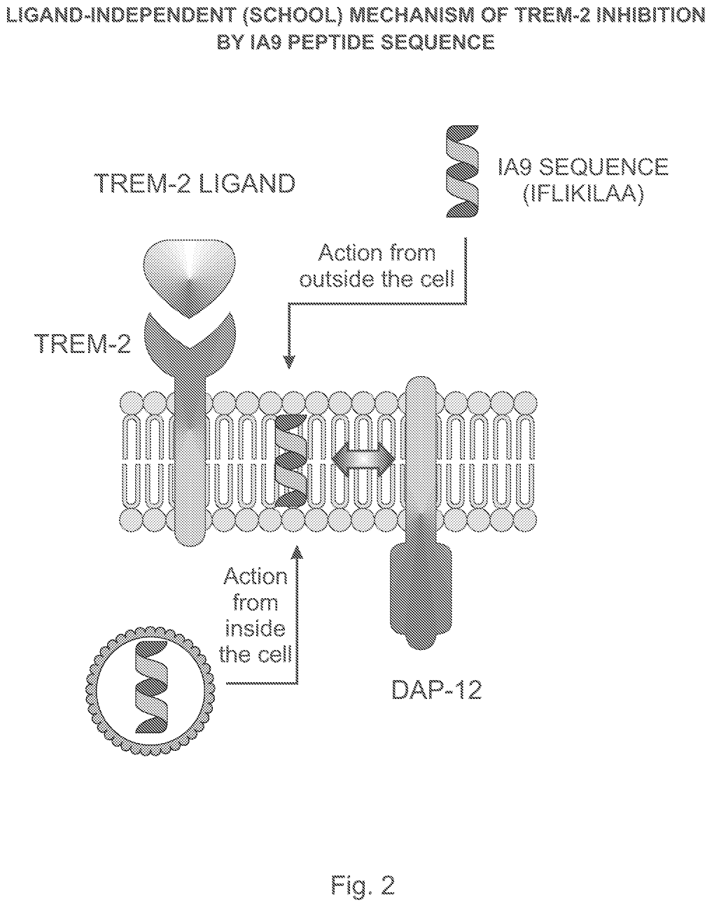

Although it is not necessary to understand the mechanism of an invention, it is believed that peptide inhibitors and compositions as described herein have a site of action in the cell membrane and advantageously employ ligand-independent mechanisms of receptor inhibitory action (see, for example, FIGS. 1, 2). It is further believed that, due to their intramembrane site of action, these peptide inhibitors and compositions can reach this site from both outside and inside the cell (see FIGS. 1, 2).

In one embodiment, a TREM-1 inhibitor peptide sequence GFLSKSLVF (GF9) delivered in a free peptide form represents “pan” TREM-1 inhibitor that inhibits TREM-1 on all TREM-1-expressing cells, while GF9 peptide sequence delivered in a form of lipopeptide complexes (LPC) is targeted TREM-1 inhibitor that inhibits TREM-1 preferentially on those cells into which it is delivered by using LPC.

In one embodiment, a TREM-2 inhibitor peptide sequence IFLIKILAA (IA9) delivered in a free peptide form represents “pan” TREM-2 inhibitor that inhibits TREM-2 on all TREM-2-expressing cells, while IA9 peptide sequence delivered in a form of lipopeptide complexes (LPC) of the invention is targeted TREM-2 inhibitor that inhibits TREM-2 preferentially on those cells into which it is delivered by using LPCs.

In one embodiment, a combinatorial TREM-1 and TREM-2 inhibitor peptide sequence GFLSKSLVFIFLIKILAA (GA18) delivered in a free peptide form represents “pan” TREM-1 and TREM-2 inhibitor that inhibits TREM-1 and TREM-2 concurrently on all TREM-1 and/or TREM-2-expressing cells, while peptide sequence GA18 delivered in a form of lipopeptide complexes (LPC) of the invention is targeted TREM-1 and TREM-2 concurrent inhibitor that inhibits TREM-1 and/or TREM-2 preferentially on those cells into which it is delivered by using LPCs.

### II. TREM-1 and TREM-2: Assembly, Signaling and Inhibitor Peptides

### A. TREM-1

TREM-1 is believed to be expressed on a majority of innate immune cells and, to a lesser extent, on parenchymal cells. TREM-1 has been reported to amplify inflammation and be upregulated in inflammatory conditions. (Bouchon et al. 2001, Gibot 2006b, Palazzo et al. 2012, Pelham et al. 2014, Tammaro et al. 2017, Sigalov 2020, Ford et al. 2021, de Oliveira et al. 2022) and disclosed in US 2020/0254058; U.S. Pat. Nos. 8,513,185; 9,981,004; 10,603,357; US 2019/0117725; and US 2021/0322508 (all of which are herein incorporated by reference). TREM-1 mediates release of MCP-1, TNFa, IL-1b, IL-6 and CSF-1.

TREM-1 blockade has been reported as an approach to sepsis and other inflammatory disorders. See, for example, (Schenk et al. 2007, Dower et al. 2008, Pelham et al. 2014, Qian et al. 2014, Sigalov 2014b, Shen et al. 2017b, Rojas et al. 2018, Tornai et al. 2019, Sigalov 2020, Gallop et al. 2021).

TREM-1 assembly, signaling and ligand-independent inhibition using peptide variants and compositions designed based on the SCHOOL model of MIRR signaling are described in detail (Sigalov 2004, Sigalov 2006, Sigalov 2010b, Sigalov 2010a, Sigalov 2020) and disclosed in U.S. Pat. Nos. 8,513,185; 9,981,004; US 2019/0117725; and US 2021/0322508 (all of which are herein incorporated by reference). Nature of specific TREM-1 cognate ligand(s) is currently not well understood (Tammaro et al. 2017) which makes advantageous use of the ligand-independent TREM-1 inhibitor peptide variants and compositions described herein and those described in (Sigalov 2020) and disclosed in U.S. Pat. No. 11,097,020; US 2021/0322508; US 2022/0047512; and US 20110256224 (all of which are herein incorporated by reference); and WO 2020/036987; as compared to other, ligand-dependent TREM-1 inhibitors such as peptides RGFFRGG (M3), LQEEDAGEYGCM (LR12) and LQVTDSGLYRCVIYHPP (LP17) or anti-TREM-1 blocking antibody. See, for example, (Gibot et al. 2006b, Gibot et al. 2007, Gibot et al. 2009, Zhou et al. 2013, Brynjolfsson et al. 2016, Joffre et al. 2016, Cuvier et al. 2018, Denning et al. 2020a, Denning et al. 2020b, Denning et al. 2020c, Siskind et al. 2022). These ligand-dependent TREM-1 inhibitors all attempt to block binding of currently uncertain ligands of TREM-1 and have a risk of failure in clinics.

Targeted delivery of the peptide variants and compositions of this disclosure by using HDL-mimicking LPC (or SLP, or LNP) has been also described in (Sigalov 2014b, Shen and Sigalov 2017b, Shen et al. 2017a, Rojas et al. 2018, Mark et al. 2019, Tornai et al. 2019, Sigalov 2020) and disclosed in U.S. Pat. No. 11,097,020; US 2021/0322508; US 2022/0047512; and US 20110256224 (all of which are herein incorporated by reference); and WO 2020/036987. See FIG. 1.

### B. TREM-2

TREM-2 receptor is believed to be a member of the MIRR family and expressed on the surface of myeloid cells as well as on the microglia of the central nervous system and has been shown to play both immune and non-immune functions (Deczkowska et al. 2020). The ligands of TREM-2 encompass a wide array of anionic molecules, free and bound to the plasma membrane, including bacterial products, DNA, lipoproteins, and phospholipids.

TREM-2 has been reported to be mainly expressed in myeloid cells which are the major components of the tumor microenvironment. Elevated TREM-2 expression correlate with tumor progression and poor patient survival in gastric cancer, glioma, and hepatocellular carcinoma. TREM-2 is expressed in tumor macrophages in over 200 human cancer cases and inversely correlates with prolonged survival for two types of cancer: colorectal carcinoma (CRC) and triple-negative breast cancer (TNBC) (Molgora et al. 2020). In cancer mouse models, administration of an Fc-mutated anti-TREM2 monoclonal antibody (mAb) to tumor-bearing mice blunts tumor growth, strongly enhances the efficacy of anti-programmed cell death protein 1 (PD-1) immunotherapy and protects sarcoma-modeling mice (Molgora et al. 2020). Although it is not necessary to understand the mechanism of an invention, it is beleved that one modus operandi in cancer is to block TREM-2 signaling or deplete TREM-2+ myeloid cells from the tumor microenvironment, allowing reactivation of the T cell-mediated anti-tumor immune response (Deczkowska et al. 2020).

While a role of TREM-2 and therapeutic effect of its blockade in cancer are relatively well understood (Molgora et al. 2020, Binnewies et al. 2021, Cheng et al. 2021, Qiu et al. 2021), the data on its role in sepsis, lung inflammation, autoimmune and other inflammation-associated diseases and conditions are either not available or controversial (Chen et al. 2013, Gawish et al. 2015, Weehuizen et al. 2016, Sun et al. 2019, Wang et al. 2019, Zhu et al. 2019).

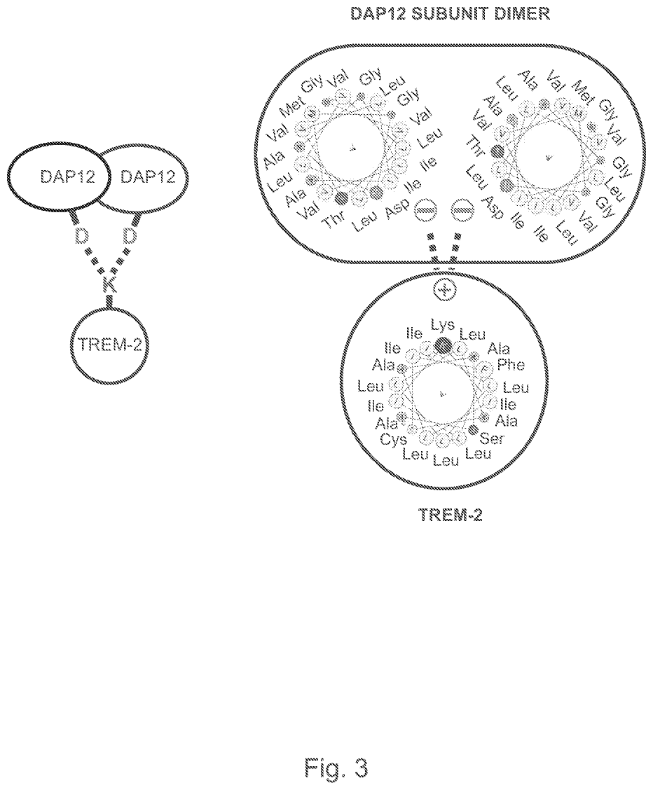

TREM-2 signals through DAP-12 signaling subunit noncovalently associated with TREM-2 subunit in the membrane. Normal transmembrane interactions between the TREM-2 and the DAP-12 dimer forming a functional TREM-2/DAP-12 receptor complex comprise a positively charged lysine amino acid within the TREM-2 transmembrane portion and negatively charged aspartic acid pairs in a DAP-12 dimer, thereby allowing subunit association. See FIG. 3.

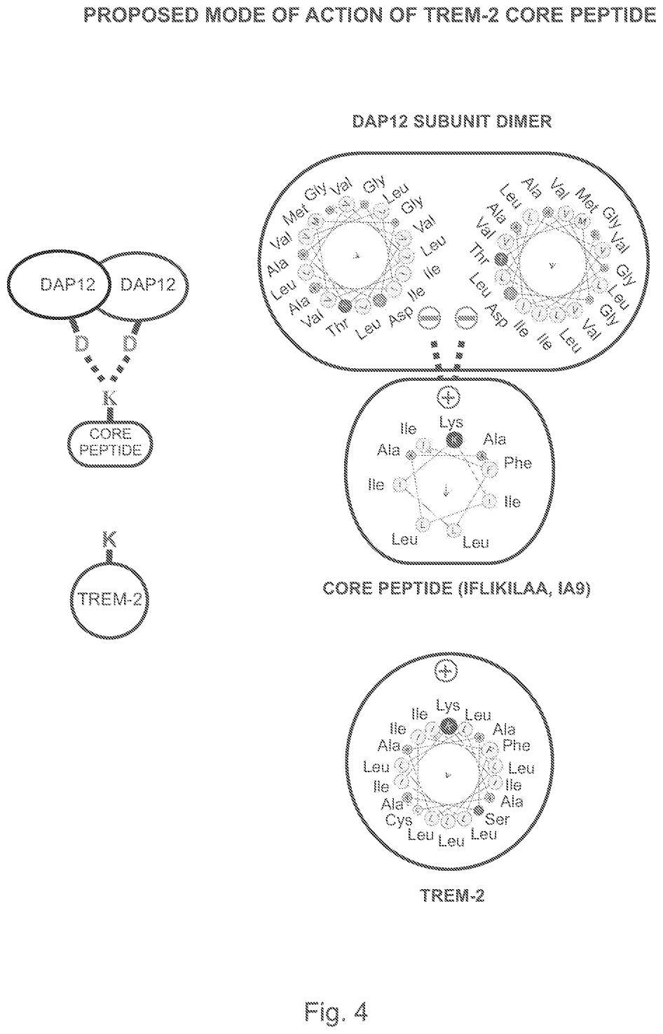

Although it is not necessary to understand the mechanism of an invention, it is believed that interactions between a lysine residue of a TREM-2 core or extended peptide inhibitor and an aspartic acid residue of a DAP-12 dimer disrupt the interactions between TREM-1 and DAP-12 in the cell membrane, thereby “disconnecting” TREM-2 and resulting in a “pre-dissociated” non-functioning receptor. See FIGS. 2, 4.

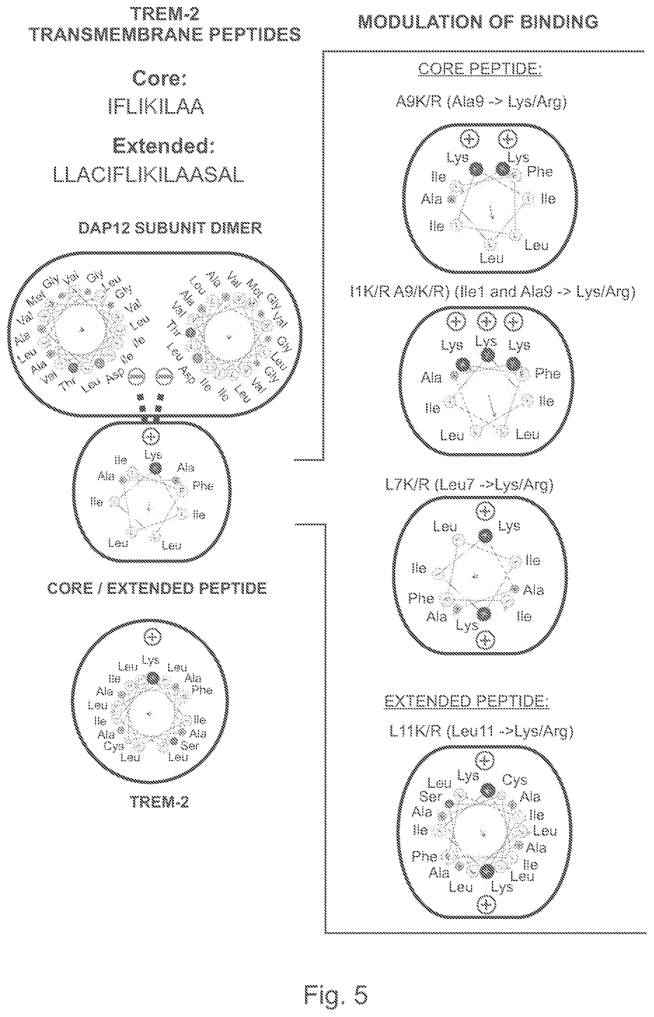

A TREM-2 inhibitor core peptide comprises, consists essentially of, or consists of a peptide having the sequence IFLIKILAA (SEQ. ID NO: 2). See FIGS. 4, 5.

A TREM-2 inhibitor extended peptide comprises, consists essentially of, or consists of a peptide having the sequence LLACIFLIKILAASAL. See FIG. 5.

In one embodiment, the present invention contemplates a series of peptides that are inhibitors of a TREM receptor (i.e., for example, a TREM-2/DAP-12 complex) Although it is not necessary to understand the mechanism of an invention, it is believed that this inhibition is mediated by disrupting the intramembrane interactions between the recognition, TREM-1, and signaling, DAP-12, subunits. In other embodiments, these peptide inhibitors treat and/or prevent diseases and/or conditions comprising activation of TREM-expressing cells. In one embodiment, the peptide inhibitors modulate TREM-1-mediated cell activation. In one embodiment, the peptide inhibitors modulate TREM-2-mediated cell activation. In another embodiment, the present invention contemplates a drug delivery system comprising inhibitor peptide variants and compositions as described herein (e.g., as disclosed in U.S. Pat. Nos. 8,513,185; 9,981,004; US 20190117725; US 2022/0047512; and US 2021/0322508 and incorporated herein by reference in their entireties); and WO 2020/036987. This drug delivery composition can also comprise nanoparticulate LPC (or synthetic lipopeptide particles, SLP) wherein said complexes comprise at least one modified apolipoprotein or its fragment and at least one lipid. Although it is not necessary to understand the mechanism of an invention, it is believed that the peptide inhibitor drug delivery system functions by penetrating the cell membrane.

Although it is not necessary to understand the mechanism of an invention, it is believed that a hydrophobic/polar/charged amino acid sequence patterning, rather than sequence similarity, within the transmembrane TREM-2 domain plays a dominant role in the development of effective peptide-based inhibitors of TREM-2-mediated cell activation. Sequence-based rational design can be used as a tool in order to increase the effectiveness of the peptides to inhibit the function of the TREM-2/DAP-12 receptor complex. For example, a conservative amino acid substitution of lysine for arginine or insertion of at least one supplemental positively charged amino acid residue (i.e., for example, arginine and/or lysine) may be made in certain locations on a-helixes of TREM-2 core or extended peptides. Although it is not necessary to understand the mechanism of an invention, it is believed that these changes should result in increased binding activity to the transmembrane domain of the DAP-12 signaling subunit dimer, thus enhancing the effectiveness of the peptides to inhibit the function of an TREM-2/DAP-12 receptor complex. See FIG. 5.

In some embodiments, as contemplated by the present invention, optimal peptide inhibitors and peptide inhibitor analogues are designed using hydrophobic/polar/charged sequence pattern criteria and associated evaluation techniques. These peptide inhibitors may then be synthesized and tested in cell function inhibition assays and in animal studies.

Substituted amino acid residues in the TREM-2 inhibitor peptide variants of the invention can be unrelated to the amino acid residue being replaced (e.g., unrelated in terms of hydrophobicity/hydrophilicity, size, charge, polarity, etc.), or the substituted amino acid residues can constitute similar, conservative, or highly conservative amino acid substitutions. As disclosed in U.S. Pat. No. 10,016,489 (herein incorporated by reference) and used herein, “similar”, “conservative”, and “highly conservative” amino acid substitutions are defined as shown in the TABLE 1, below. The determination of whether an amino acid residue substitution is similar, conservative, or highly conservative is based exclusively on the side chain of the amino acid residue and not the peptide backbone, which may be modified to increase peptide stability, as discussed below.

TABLE 1
Amino Acid Substitutions

| | | Conservative | Highly Conservative |
|---|---|---|---|
| Amino | Similar Amino | Amino Acid | Amino Acid |
| Acid | Acid | Substitution | Substitution |
| Ala (A) | S, G, T, V, C, P, Q | S, G, T | S |
| Arg (R) | K, H | K, H | K, H |
| Asn (N) | Q | Q | Q |
| Asp (D) | E | E | E |
| Cys (C) | A, S, T, V, I | A | n/a |
| Gln (Q) | N | N | N |
| Glu (E) | D | D | D |
| Gly (G) | A, S, N | A | n/a |
| His (H) | R, K | R, K | R, K |
| Ile (I) | V, L, M, F, T, C | V, L, M, F | V, L, M |
| Leu (L) | M, I, V, F, T, A | M, I, V, F | M, I |
| Lys (K) | R, H | R, H | R, H |
| Met (M) | L, I, V, F | L, I, V | L, I |
| Phe (F) | W, L, M, I, V | W, L | n/a |
| Pro (P) | A, S, T | A | n/a |
| Ser (S) | T, A, N, G, Q | T, A, N | T, A |
| Thr (T) | S, A, V, N, M | S, A, V, N | S |
| Trp (W) | F, L, V | F | n/a |
| Tyr (Y) | F, W, H, L, I | F, W | F |
| Val (V) | I, L, M, T, A | I, L, M | I |

In some embodiments, the peptide inhibitors comprise D-stereoisomeric amino acids, thereby allowing the formulation of immunotherapeutic peptides with increased resistance to protease degradation. In one embodiment, the D-amino acid peptide inhibitors are used for the clinical treatment in myeloid cell-mediated disorders. Although it is not necessary to understand the mechanism of an invention, it is believed that these peptide inhibitors prevent activation of TREM-2-expressing myeloid cells.

In some embodiments, the present invention contemplate peptide inhibitors that are protease resistant. In one embodiment, such protease-resistant peptide inhibitors are peptides comprising protecting groups. For example, a peptide may be protected from exoproteinase degradation by N-terminal acetylation (“Ac”) and/or C-terminal amidation.

In some embodiments, the peptide inhibitors further comprise conjugated lipids and/or sugars. In other embodiments, the peptide inhibitors further comprise hydrophobic amino acid motifs, wherein said motifs are believed to increase the membrane penetrating ability of peptides and proteins. Although it is not necessary to understand the mechanism of an invention, it is believed that either lipid/sugar conjugation and/or hydrophobic amino acid motifs increase the efficacy of TREM-2 inhibition using either TREM-2 Core Peptides and/or Extended Peptides.

In some embodiments, the peptides and compounds as contemplated by the present invention may be used for production of peptide/compound-containing implants or implantable devices.

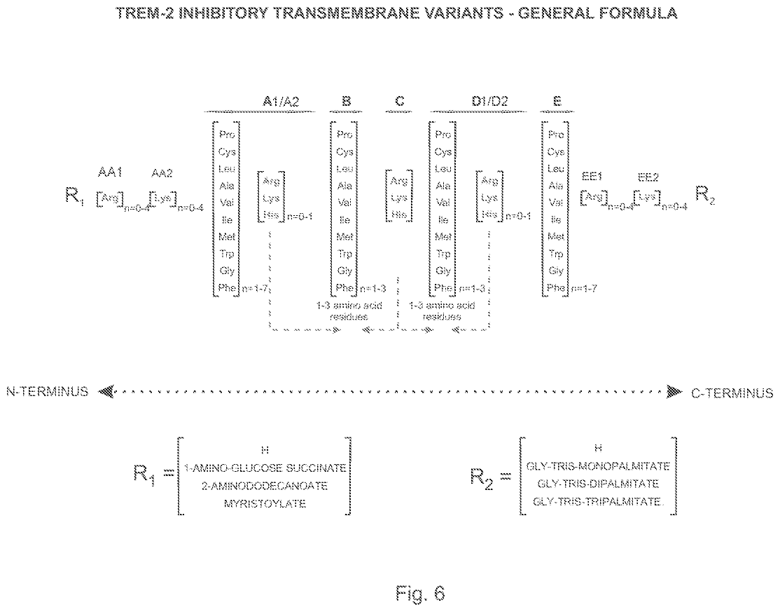

In one embodiment, the present invention contemplates a TREM-2 inhibitor peptide having the general formula R₁-A-B-C-D-E-R₂ (See FIG. 6) or a disulfide-bridged, linear dimer thereof, or a cyclic dimer thereof, wherein:
A is a peptide consisting of 1 to 7 hydrophobic uncharged D- or L-amino acids, or a peptide consisting of 1 to 6 hydrophobic uncharged D- or L-amino acids surrounding a positively charged D- or L-amino acid which is spaced 1 to 3 amino acids from C;
B is a peptide consisting of 1 to 3 hydrophobic uncharged D- or L-amino acids, including D- or L-cysteine or a D- or L-cysteine homologue;
C is a positively charged D- or L-amino acid;
D is a peptide consisting of 1 to 3 hydrophobic uncharged D- or L-amino acids, or a peptide consisting of 1 to 3 hydrophobic uncharged D- or L-amino acids surrounding a positively charged D- or L-amino acid which is spaced 1 to 3 amino acids from C;
E is a peptide consisting of 1 to 7 hydrophobic uncharged D- or L-amino acids;
R₁ is absent (i.e., for example, —H) or 1-amino-glucose succinate, 2-aminododecanoate, or myristoylate; and
R₂ is absent (i.e., for example, —H) or Gly-Tris-monopalmitate, -dipalmitate and -tripalmitate.

In some embodiments, peptide derivatives are created wherein:
A is selected from the group comprising Leu, Ala, Cys, Ile, Pro, Lys, Arg, and Phe;
B is selected from the group comprising Leu, Ala, Cys, Ile, Pro, and Phe;
C is selected from the group comprising Arg, Lys, or His;
D is selected from the group comprising Leu, Ala, Cys, Ile, Pro, Lys, Arg, and Phe;
E is selected from Leu, Ala, Cys, Ile, Pro, and Phe.

In one embodiment, the present invention contemplates a TREM-2 inhibitor peptide having the general formula R₁-[Arg and/or Lys]ₙ₌₀₋₄-A-B-C-D-E-[Arg and/or Lys]ₙ₌₀₋₄-R₂ or a disulfide-bridged, linear dimer thereof, or a cyclic dimer thereof, wherein:
A may be 1-7 amino acids selected from the group including, but not limited to, Pro, Cys, Leu, Ala, Val, Ile, Met, Trp, Gly, Phe, Lys, or Arg;
B may be 1-3 amino acids selected from the group including, but not limited to, Pro, Cys, Leu, Ala, Val, Ile, Met, Trp, Gly, and Phe;
C may be selected from the group including, but not limited to, Arg, Lys, and His;
D may be 1-3 amino acids selected from the group including, but not limited to, Pro, Cys, Leu, Ala, Val, Ile, Met, Trp, Gly, Phe, Lys, or Arg;
E may be 1-7 amino acids selected from the group including, but not limited to, Pro, Cys, Leu, Ala, Val, Ile, Met, Trp, Gly, and Phe.
R₁ and R₂ may be either i) absent; ii) a conjugated lipid selected from the group including, but not limited to, Gly-Tris-monopalmitate, -dipalmitate and -tripalmitate; or iii) a conjugated sugar selected from the group including, but not limited to, 1-amino-glucose succinate, 2-aminododecanoate, or myristoylate. See, FIG. 6.

In one embodiment, the present invention contemplates a TREM-2 inhibitor peptide comprising an amino acid sequence having the general formula of R1-EE1-EE2-AA1-AA2-A1-A2-B-C-D1-D2-E-EE1-EE2-R2, wherein:
R1 is absent or is selected from the group consisting of N-terminal sugar conjugate and N-terminal lipid conjugate;
AA1 is absent or is selected from the group consisting of Arg, Arg-Arg, Arg-Arg-Arg and Arg-Arg-Arg-Arg;
AA2 is absent or is selected from the group consisting of Lys, Lys-Lys, Lys-Lys-Lys and Lys-Lys-Lys-Lys;
A1 is an amino acid selected from the group consisting of Pro, Cys, Leu, Ala, Val, Ile, Met, Trp, Gly and Phe, a two amino acid peptide, a three amino acid peptide, a four amino acid peptide, a five amino acid peptide, a six amino acid peptide and a seven amino acid peptide, said peptide consisting of Pro, Cys, Leu, Ala, Val, Ile, Met, Trp, Gly and Phe in any combination;
A2 is absent or is a positively charged amino acid selected from the group comprising Arg, Lys and His;
B is selected from the group consisting of Pro, Cys, Leu, Ala, Val, Ile, Met, Trp, Gly and Phe, a two amino acid peptide and a three amino acid peptide, said peptide consisting of Pro, Cys, Leu, Ala, Val, Ile, Met, Trp, Gly and Phe in any combination;
C is a positively charged amino acid selected from the group comprising Arg, Lys and His;
D1 is selected from the group consisting of Pro, Cys, Leu, Ala, Val, Ile, Met, Trp, Gly and Phe, a two amino acid peptide and a three amino acid peptide, said peptide consisting of Pro, Cys, Leu, Ala, Val, Ile, Met, Trp, Gly and Phe in any combination;
D2 is absent or is a positively charged amino acid selected from the group comprising Arg, Lys and His;
E is an amino acid selected from the group consisting of Pro, Cys, Leu, Ala, Val, Ile, Met, Trp, Gly and Phe, a two amino acid peptide, a three amino acid peptide, a four amino acid peptide, a five amino acid peptide, a six amino acid peptide and a seven amino acid peptide, said peptide consisting of Pro, Cys, Leu, Ala, Val, Ile, Met, Trp, Gly and Phe in any combination;
EE1 is absent or is selected from the group consisting of Arg, Arg-Arg, Arg-Arg-Arg and Arg-Arg-Arg-Arg;
EE2 is absent or is selected from the group consisting of Lys, Lys-Lys, Lys-Lys-Lys and Lys-Lys-Lys-Lys; and
R2 is absent or is C-terminal lipid conjugate. See, FIG. 6.

As referred to herein, hydrophobic amino acids include, but are not limited to, Ala, Val, Leu, Ile, Pro, Phe, Trp, and Met; positively charged amino acids include, but are not limited to, Lys, Arg and His; and negatively charged amino acids include, but are not limited to, Asp and Glu.

The general formula above represents one embodiment of a TREM-2 transmembrane segment comprising at least one conserved domain that contains highly homologous sequences between species. In one embodiment, a TREM-2 transmembrane segment comprises IA9 (Ile-Phe-Leu-Ile-Lys-Ile-Leu-Ala-Ala; human amino acid residues 182-190; Accession No. Q9NZC2) along with various lipid and/or sugar derivatives that may, or may not, have a disulfide bridged dimer. In another embodiment, a TREM-2 transmembrane segment comprises KKLLLACIFLIKILAASALWAKR, wherein said sequence meets the criteria for the above outlined general formula.

In one embodiment, the present invention contemplates a method of rational designing of the peptides and lipid- and/or sugar-conjugated peptides consisting of L- or D-stereoisomeric amino acids in order to increase effectiveness of the peptides in inhibiting the function of a TREM-2/DAP-12 receptor complex. In one embodiment, the method comprises substituting at least one amino acid of a TREM-2 transmembrane core or extended peptide (i.e., for example, and arginine or a lysine into at least one alpha-helix of the Core Peptide and/or Extended Peptide), thereby increasing binding to the transmembrane domain of DAP-12 chain. See FIG. 5.

In another embodiment, the method comprises conjugating at least one lipid and/or at least one sugar to the C- and/or N-termini of the peptide, thereby increasing binding to the transmembrane domain of the DAP-12 chain and/or improving the penetration of the peptide variant into the cell membrane. In one embodiment, the lipid- and/or sugar-conjugated peptide variants comprise D-amino acids, thereby increasing resistance to protease degradation. In one embodiment, a protease resistant peptide variant is useful clinically for inhibiting TREM-mediated cell activation in myeloid cell-mediated disorders.

In some embodiments, conjugated peptide variants are synthesized using the standard procedures as described, for example, in (Ali et al. 2005, Amon et al. 2006) and disclosed in U.S. Pat. No. 7,192,928; US 20120077732; US 20100267651; and US 20050070478 (all of which are herein incorporated by reference).

In one embodiment, the rational design method comprises inserting at least one polyarginine and/or polylysine sequence into a TREM-2 transmembrane sequence, thereby increasing binding to a transmembrane domain of an DAP-12 chain and/or improving the penetration of the peptide variant into the cell membrane. Other modifications of the peptides contemplated herein include, but are not limited to, modifications to side chains, incorporation of unnatural amino acids and/or their derivatives during peptide synthesis and the use of crosslinkers and other methods which impose conformational constraints on the peptides. It may also be possible to add various groups to the peptide of the present invention to confer advantages such as increased potency or extended half life in vivo without substantially decreasing the biological activity of the peptide. It is intended that such modifications to the peptide of the present invention which do not result in a decrease in biological activity are within the scope of the present invention.

Any combination of the above embodiments may be used together in order to increase effectiveness of the peptide variants to inhibit the function of a TREM-2/DAP-12 receptor complex. The most effective inhibitory peptides and derivatives thereof may be identified by typical screening assay procedures for evaluation of inhibition of TREM-mediated cell activation and function (Klesney-Tait et al. 2006, Ford et al. 2009).

A list of the sequences of the peptides and peptide analogues shown below includes, but is not limited to, peptide-based inhibitors proved and predicted to be effective in inhibiting the TREM-2/DAP-12 signaling. See TABLE 2.

TABLE 2
Exemplary Peptide-Based TREM-2/DAP-12
Receptor Complex Inhibitor Sequences

| | | Sequence | |
|---|---|---|---|
| SEQ. | | (the “core” sequence of the | |
| ID | | peptide of the invention | |
| NO | R₁ª | is underlined) | R₂^{b} |
| 1 | — | ILLLLACIFLIKILAASALWA (parent) | − |
| | | (TREM-2 TM peptide) | |
| 2 | — | IFLIKILAA | − |
| | | (TREM-2 TM core peptide) | |
| 3 | — | ILLLLACIFLIKILAASALWA | + |
| 4 | LA | ILLLLACIFLIKILAASALWA | − |
| 5 | Myr | GSLLLLACIFLIKILAASALWA | − |
| 6 | LA | ILLLLACIFLIKILAASALWA | + |
| 7 | — | KKLLLACIFLIKILAASALWAKR | − |
| 8 | — | KKLLLACIFLIKILAASALWAKR | + |
| 9 | — | (ILLLLACIFLIKILAASALWA)₂^{c} | − |
| 10 | — | ILLLLACIFLIKIKAASALWA | − |
| 11 | — | (ILLLLAC*IFLIKILAASALWA)₂^{d} | − |
| 12 | — | ILLLLACIFLIKILARSALWA | − |
| 13 | — | ILLLLACIFLIKILARSALWA | + |
| 14 | LA | ILLLLACIFLIKILARSALWA | − |
| 15 | Myr | GSLLLACIFLIKILARSALWA | − |
| 16 | — | KKLLLACIFLIKILARSALWAKR | − |
| 17 | LA | KKLLLACIFLIKILARSALWAKR | − |
| 18 | — | KKLLLACIFLIKILARSALWAKR | + |
| 19 | — | (ILLLLACIFLIKILARSALWA)₂ | − |
| 20 | — | ILLLLAGIFLIKILARSALWA | − |
| 21 | — | (ILLLLAC*IFLIKILARSALWA)₂ | − |
| 22 | — | ILLLLACRFLIKILARSALWA | − |
| 23 | LA | ILLLLACRFLIKILARSALWA | − |
| 24 | — | ILLLLACRFLIKILARSALWA | + |
| 25 | — | KKLLLACRFLIKILARSALWAKR | − |
| 26 | LA | KKLLLACRFLIKILARSALWAKR | − |
| 27 | — | KKILLLLACRFLIKILARSALWAKR | + |
| 28 | — | (ILLLLACRFLIKILARSALWA)₂ | − |
| 29 | — | ILLLLAGRFLIKILARSALWA | − |
| 30 | — | (ILLLLAC*RFLIKILARSALWA)₂ | − |
| 31 | — | IFLIKILAA | + |
| 32 | LA | IFLIKILAA | − |
| 33 | LA | IFLIKILAA | + |
| 34 | — | (IFLIKILAA)₂ | − |
| 35 | — | ACIFLIKILAA | − |
| 36 | — | (AC*IFLIKILAA)₂ | − |
| 37 | — | IFLIKILAR | − |
| 38 | LA | IFLIKILAR | − |
| 39 | — | IFLIKILAR | + |
| 40 | — | (IFLIKILAR)₂ | − |
| 41 | — | ACIFLIKILAR | − |
| 42 | — | (AC*IFLIKILAR)₂ | − |
| 43 | — | KFLIKILAR | − |
| 44 | LA | KFLIKILAR | − |
| 45 | — | KFLIKILAR | + |
| 46 | — | (KFLIKILAR)₂ | − |
| 47 | — | ACKFLIKILAR | − |
| 48 | — | (AC*KFLIKILAR)₂ | − |

^{a}N-terminal group: LA, lipoamino acid, 2-aminododecanoate; Myr, myristoylate.
^{b}C-terminal group: Gly-Tris-tripalmitate.
^{c}Cyclic peptide.
^{d}Disulfide-linked dimer (or disulfide-linked cyclic dimer).
*Cys involved in disulfide bond formation.
TM = transmembrane

### C. Combinatorial TREM-1 and TREM-2 Inhibitor Peptides

In one embodiment, combinatorial receptor inhibitor peptide variants and compositions can be designed to target two or more MIRR receptors concurrently. In one embodiment, said MIRRs are TREM-1 and TREM-2. In one embodiment, said TREM-1 and TREM-2 are expressed on the same cell. In one embodiment, said TREM-1 and TREM-2 are expressed on different cells. In one embodiment, TREM-1 inhibitor domain of the combinatorial TREM-1 and TREM-2 inhibitor peptide variants and compositions comprises peptide variants and compositions in detail (Joffre et al. 2016, Denning et al. 2020a, Denning et al. 2020b, Denning et al. 2020c, Francois et al. 2020, Sigalov 2020, Shen et al. 2021, Wang et al. 2021a, Wang et al. 2021b, Siskind et al. 2022) and disclosed in U.S. Pat. Nos. 8,513,185; 9,981,004; 9,255,136; 9,657,081; 9,815,883; 10,765,679; 11,052,090 US 20190117725; and US 20210322508 (all of which are herein incorporated by reference).

In one embodiment, a TREM-2 inhibitor domain of said combinatorial TREM-1 and TREM-2 inhibitor peptide variants and compositions comprises a plurality of TREM-2 inhibitor peptide variants and compositions of the present invention.

In one embodiment, a TREM-1 inhibitor domain is located at the N-terminus of said combinatorial TREM-1 and TREM-2 inhibitor peptide variants and compositions. In one embodiment, a TREM-1 inhibitor domain is located at the C-terminus of said TREM-1 and TREM-2 combinatorial inhibitor peptide variants and compositions.

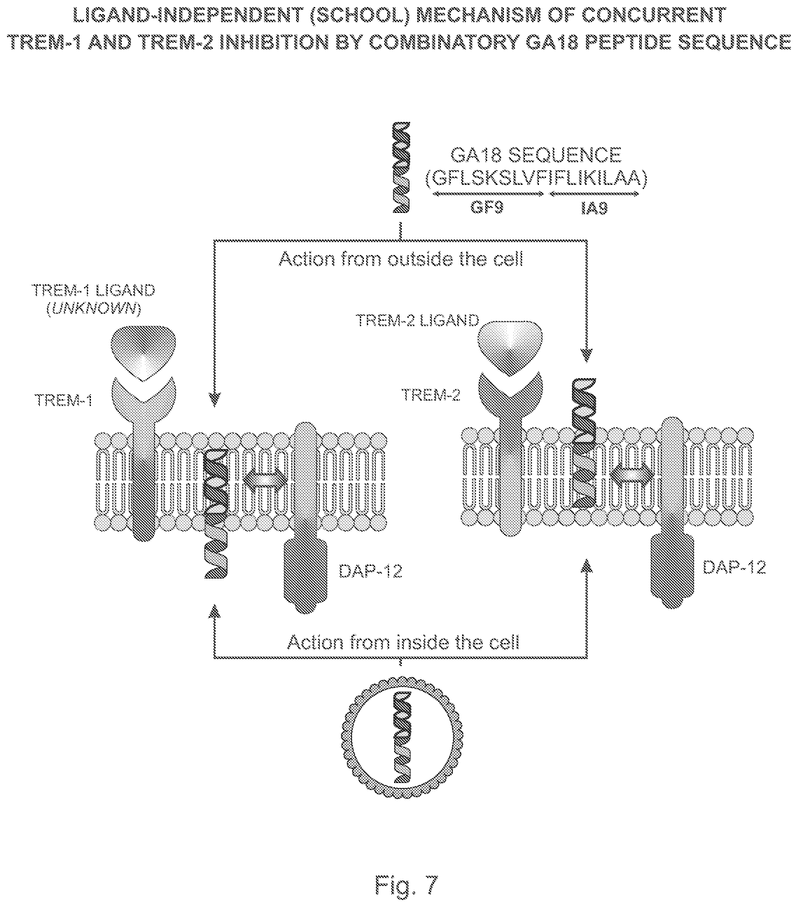

In one embodiment, said combinatorial TREM-1 and TREM-2 concurrent inhibitor peptide sequence comprises the amino acid sequence of GFLSKSLVFIFLIKILAA (GA-18) that comprises a 9 amino acid-long TREM-1 inhibitor sequence GF9 as the N-terminus and a 9 amino acid-long TREM-2 inhibitor sequence IA9 as the C-terminus. In one embodiment, the combinatorial TREM-1 and TREM-2 concurrent inhibitor peptide sequence is administered in a form of free peptide. In one embodiment, the combinatorial TREM-1 and TREM-2 concurrent inhibitor peptide sequence is administered in a form of targeted LPC. Although it is not necessary to understand the mechanism of an invention, it is believed that when delivered in a free peptide form, combinatorial TREM-1 and TREM-2 concurrent inhibitor peptide sequence inhibits TREM-1 and TREM-2 on all TREM-1- and/or TREM-2-expressing cells (“pan” TREM-1 and TREM-2 inhibitor), while when delivered by targeted LPC, combinatorial TREM-1 and TREM-2 concurrent inhibitor peptide sequence inhibits TREM-1 and TREM-2 preferentially on those TREM-1- and/or TREM-2-expressing cells, into which it is delivered (targeted TREM-1 and TREM-2 inhibitor). See FIG. 7.

In certain embodiments, the preferred combinatorial TREM-1 and TREM-2 concurrent inhibitor peptide variants and compositions of the invention include but are not limited to TREM-1 inhibitory peptide sequences such as GFLSKSLVF (GF9), RGFFRGG (M3), LQEEDAGEYGCM (LR12) and LQVTDSGLYRCVIYHPP (LP17) described in (Gibot et al. 2006b, Gibot et al. 2007, Gibot et al. 2009, Zhou et al. 2013, Joffre et al. 2016, Cuvier et al. 2018, Denning et al. 2020a, Denning et al. 2020b, Denning et al. 2020c, Sigalov 2020, Siskind et al. 2022) and disclosed in U.S. Pat. Nos. 8,013,116; 9,273,111; 9,657,081; 9,815,883; 10,603,357; 9,255,136; 8,513,185; 9,981,004; US 2019/0117725; and US 2021/0322508 (all of which are herein incorporated by reference). See TABLE 3.

### D. Other Combinatorial Cell Receptor Inhibitor Peptides

Also contemplated as embodiments of the inventive methods are peptide variants and compositions thereof that are designed as combinatorial inhibitor peptide variants and compositions to inhibit two or more cell surface receptors. In one embodiment, these peptide variants and compositions employ a ligand-independent (SCHOOL) mechanism of inhibition of receptor signaling (Sigalov 2006, Sigalov 2020). In one embodiment, said cell surface receptors include, but are not limited to, BCR, CLR, DCAR, GPVI, ILT family members; KIR, LIR family of receptors, MAIR-II, MDL-1, NITR, NKCRs (KIR2DS, NKG2D, NKp46, NKp44, NKp30, etc), SIRP family of receptors, TCR, TREM family of receptors (TREM-1, TREM-2, TREM3, TREM-4). In one embodiment, combinatorial inhibitor peptide variants and compositions concurrently inhibit two or more receptors expressed on the surface of the same cell (e.g., TREM-1 and TREM-2 on cells that express both these receptors) or on the surface of different cells (e.g., TCR and TREM-1).

In one embodiment, targeted receptors are identified by using combinatorial inhibitor peptide variants and compositions as described herein that are based on a role and involvement of cells expressing these receptors in the pathogenesis of a particular disease or condition. For example, for rheumatoid arthritis (RA) in which TCR-, TREM-1- and/or TREM-2-expressing cells are all involved in the pathogenesis. In one embodiment, a combinatorial TREM-1 and TREM-2 inhibitor peptide sequence GFLSKSLVFIFLIKILAA (GA18; SEQ. ID NO: 49) can be used to target TREM-1 and TREM-2 simultaneously in treating RA. In one embodiment, a combinatorial TCR and TREM-1 inhibitor peptide sequence GFRILLLKVGFLSKSLVF (SEQ. ID NO: 54) can be used to target TCR and TREM-1 simultaneously in treating RA. In one embodiment, a combinatorial TCR and TREM-1 inhibitor peptide sequence MWKTPTLKYFGFLSKSLVF (SEQ. ID NO: 56) can be used to target TCR and TREM-1 simultaneously in treating RA. In one embodiment, a combinatorial TCR and TREM-2 inhibitor peptide sequence GFRILLLKVIFLIKILAA (SEQ. ID NO: 58) can be used to target TCR and TREM-2 simultaneously in treating RA. In one embodiment, a combinatorial TCR and TREM-2 inhibitor peptide sequence MWKTPTLKYFIFLIKILAA (SEQ. ID NO: 61) can be used to target TCR and TREM-2 simultaneously in treating RA. In one embodiment, a combinatorial TCR, TREM-1 and TREM-2 inhibitor peptide sequence GFRILLLKVGFLSKSLVFIFLIKILAA (SEQ. ID NO: 60) can be used to target TCR, TREM-1 and TREM-2 simultaneously in treating RA. In one embodiment, a combinatorial TCR, TREM-1 and TREM-2 inhibitor peptide sequence MWKTPTLKYFGFLSKSLVFIFLIKILAA (SEQ. ID NO: 63) can be used to target TCR, TREM-1 and TREM-2 simultaneously in treating RA. In one embodiment, other TCR inhibitor peptide sequences can be used in TCR combinatorial inhibitor peptide sequences leading to selective inhibition of TCR signaling mediated by different signaling subunits of TCR-CD3 receptor complex (e.g., TCRz, CD3e, CD3g, and/or CD3d signaling subunits) as described in (Collier et al. 2006, Sigalov 2010a, Sigalov 2020). See TABLE 3. In one embodiment, a TCR inhibitor peptide sequence is located at the N-terminus of the combinatorial inhibitor peptide. In one embodiment, a TCR inhibitor peptide sequence is located at the C-terminus of the combinatorial inhibitor peptide.

In one embodiment, the present invention contemplates a cancer disease in which TREM-1 and/or TREM-2-expressing cells are involved in the pathogenesis of the cancer disease. In one embodiment, a combinatorial TREM-1 and TREM-2 inhibitor peptide sequence GFLSKSLVFIFLIKILAA (GA18; SEQ. ID NO: 49) can be used to target TREM-1 and TREM-2 simultaneously in treating the cancer disease.

In one embodiment, is the present invention contemplates ARDS in which TCR-, TREM-1- and TREM-2-expressing cells are involved in the pathogenesis of ARDS. In one embodiment, a combinatorial TREM-1 and TREM-2 inhibitor peptide sequence GFLSKSLVFIFLIKILAA (GA18; SEQ. ID NO: 49) can be used to target TREM-1 and TREM-2 simultaneously in treating ARDS. In one embodiment, a combinatorial TCR, TREM-1 and TREM-2 inhibitor peptide sequence GFRILLLKVGFLSKSLVFIFLIKILAA (SEQ. ID NO: 60) can be used to target TCR, TREM-1 and TREM-2 simultaneously in treating ARDS.

In certain embodiments, a combinatorial TREM-1 inhibitor peptide includes, but is not limited to, GFLSKSLVF (GF9), RGFFRGG (M3), LQEEDAGEYGCM (LR12) and LQVTDSGLYRCVIYHPP (LP17) as described in (Gibot et al. 2006b, Gibot et al. 2007, Gibot et al. 2009, Zhou et al. 2013, Joffre et al. 2016, Cuvier et al. 2018, Denning et al. 2020a, Denning et al. 2020b, Denning et al. 2020c, Sigalov 2020, Siskind et al. 2022) and disclosed in U.S. Pat. Nos. 8,013,116; 9,273,111; 9,657,081; 9,815,883; 10,603,357; 9,255,136 (all of which are herein incorporated by reference).

In one embodiment, examples of combinatorial concurrent inhibitor peptide variants that inhibit two or more cell surface receptors on the same or different cells simultaneously are shown in TABLE 3.

TABLE 3
Exemplary Combinatorial Cell Receptor
Inhibitor Peptides and Compositions*

| SEQ ID | | Recep- | Recep- | Recep- |
|---|---|---|---|---|
| NO | Amino Acid Sequence | tor 1 | tor 2 | tor 3 |
| 49 | GFLSKSLVFIFLIKILAA | TREM-1 | TREM-2 | — |
| 50 | IFLIKILAAGFLSKSLVF | TREM-2 | TREM-1 | — |
| 51 | RGFFRGGIFLIKILAA | TREM-1 | TREM-2 | — |
| 52 | LQEEDAGEYGCMIFLIKILAA | TREM-1 | TREM-2 | — |
| 53 | LQVTDSGLYRCVIYHPPIFLI | TREM-1 | TREM-2 | — |
| | KILAA | | | |
| 54 | GFRILLLKVGFLSKSLVF | TCRa | TREM-1 | — |
| 55 | GFLSKSLVFGFRILLLKV | TREM-1 | TCRa | — |
| 56 | MWKTPTLKYFGFLSKSLVF | TCRa | TREM-1 | — |
| 57 | GFLSKSLVFMWKTPTLKYF | TREM-1 | TCRa | — |
| 58 | GFRILLLKVIFLIKILAA | TCRa | TREM-2 | — |
| 59 | IFLIKILAAGFLSKSLVF | TREM-2 | TCRa | — |
| 60 | GFRILLLKVGFLSKSLVFIFL | TCRa | TREM-1 | TREM-2 |
| | IKILAA | | | |
| 61 | MWKTPTLKYFIFLIKILAA | TCRa | TREM-2 | — |
| 62 | IFLIKILAAMWKTPTLKYF | TREM-2 | TCRa | — |
| 63 | MWKTPTLKYFGFLSKSLVFIF | TCRa | TREM-1 | TREM-2 |
| | LIKILAA | | | |
| 64 | LGKATLYAVGFLSKSLVF | TCRb | TREM-1 | — |
| 65 | LGKATLYAVIFLIKILAA | TCRb | TREM-2 | — |
| 66 | CYLLDGILFGFLSKSLVF | TCRz | TREM-1 | — |
| 67 | CYLLDGILFIFLIKILAA | TCRz | TREM-2 | — |
| 68 | IVIVDICITGFLSKSLVF | CD3e | TREM-1 | — |
| 69 | IVIVDICITIFLIKILAA | CD3e | TREM-2 | — |
| 70 | FLFAEIVSIGFLSKSLVF | CD3g | TREM-1 | — |
| 71 | FLFAEIVSIIFLIKILAA | CD3g | TREM-2 | — |
| 72 | IIVTDVIATGFLSKSLVF | CD3d | TREM-1 | — |
| 73 | IIVTDVIATIFLIKILAA | CD3d | TREM-2 | — |
| 74 | LLRMGLAFLVGFLSKSLVF | NKp46 | TREM-1 | — |
| 75 | LLRMGLAFLVIFLIKILAA | NKp46 | TREM-2 | — |
| 76 | GLLVAKSLVLSAGFLSKSLVF | NKp44 | TREM-1 | — |
| 77 | GLLVAKSLVLSAIFLIKILAA | NKp44 | TREM-2 | — |
| 78 | GTVLLLRAGFYAGFLSKSLVF | NKp30 | TREM-1 | — |
| 79 | GTVLLLRAGFYAIFLIKILAA | NKp30 | TREM-2 | — |
| 80 | AMGIRFIIMVAGFLSKSLVF | NKG2D | TREM-1 | — |
| 81 | AMGIRFIIMVAIFLIKILAA | NKG2D | TREM-2 | — |
| 82 | MATVLKTIVLIGFLSKSLVF | NKG2C | TREM-1 | — |
| 83 | MATVLKTIVLIIFLIKILAA | NKG2C | TREM-2 | — |
| 84 | VLIGTSVVKIPFTILLGFLSK | KIR2DS | TREM-1 | — |
| | SLVF | | | |
| 85 | VLIGTSVVKIPFTILLIFLIK | KIR2DS | TREM-2 | — |
| | ILAA | | | |
| 86 | GNLVRICLGAVGFLSKSLVF | GPVI | TREM-1 | — |
| 87 | GNLVRICLGAVIFLIKILAA | GPVI | TREM-2 | — |
| 88 | GFLSKSLVFIFLIKILAK | TREM-1 | TREM-2 | — |
| 89 | IFLIKILAKGFLSKSLVF | TREM-2 | TREM-1 | — |

*For TCR, specific inhibitor peptides can be designed to silence complete or partial TCR signaling provided by TCR zeta (TCRz)-, CD3 epsilon (CD3e)-, CD3 delta (CD3d)-, and CD3 gamma (CD3g) TCR signaling chains. TCRa and TCRb, TCR ligand-recognition chains alpha and beta, respectively.

TREM-1 inhibitor peptides, TREM-2 inhibitor peptides, combinatorial concurrent inhibitor peptides and other inhibitor peptides and compositions of the present invention can be made synthetically and may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH2(CH2)nCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Amino acid analogues may be generated by substitutional mutagenesis and retain the biological activity of the original peptides. Examples of substitutions identified as “conservative substitutions” are shown in TABLES 1 and 4. If such substitutions result in a change not desired, then other type of substitutions, denominated “exemplary substitutions” in TABLE 4, or as further described herein in reference to amino acid classes, are introduced and the products screened for their capability of executing three functions.

TABLE 4
Amino Acid Substitutions

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

### E. Trifunctional Peptides

In one embodiment, preferred inhibitor peptides and compositions further comprise at least one modified or unmodified amphipathic apo A-I and/or A-II peptide fragment capable upon interaction with lipid and/or lipid mixtures, to form LPC—particles that mimic human lipoproteins and can be spherical or discoidal as described in (Sigalov 2014b, Sigalov 2014a, Shen et al. 2016, Shen and Sigalov 2017b, Shen and Sigalov 2017a, Rojas et al. 2018, Tornai et al. 2019, Gallop et al. 2021) and disclosed in U.S. Pat. No. 11,097,020, US 20190117725; and US 2021/0322508 (all of which are herein incorporated by reference); and WO 2020/036987. The inclusion of an amphipathic apo A-I sequences in the peptides and compositions as described herein further aids the ability to provide targeted delivery to the cells of interest. In one embodiment, the cells of interest are macrophages. Although it is not necessary to understand the mechanism of an invention, it is believed that LPC sequences further aid to extend a half life of said peptides and compositions in circulation, and to cross the blood-brain barrier (BBB), blood-retinal barrier (BRB) and/or blood-tumor barrier (BTB).

In some embodiments, the present invention relates to amphipathic trifunctional peptides including at least two amino acid domains, wherein upon interaction with lipids, these peptides self-assemble into an LPC. In certain embodiments, said LPC can be prepared as described in (Sigalov 2014b, Sigalov 2014a, Shen and Sigalov 2016, Shen and Sigalov 2017b, Shen and Sigalov 2017a, Rojas et al. 2018, Tornai et al. 2019, Gallop et al. 2021) and disclosed in U.S. Pat. No. 11,097,020, US 2019/0117725; and US 20210322508 (all of which are herein incorporated by reference); and WO 2020/036987. In one embodiment, one amino acid domain mediates formation of naturally long half-life LPC and targets said LPC to the cells of interest, whereas the other amino acid domain inhibits the cell surface receptor expressed on the cells of interest.

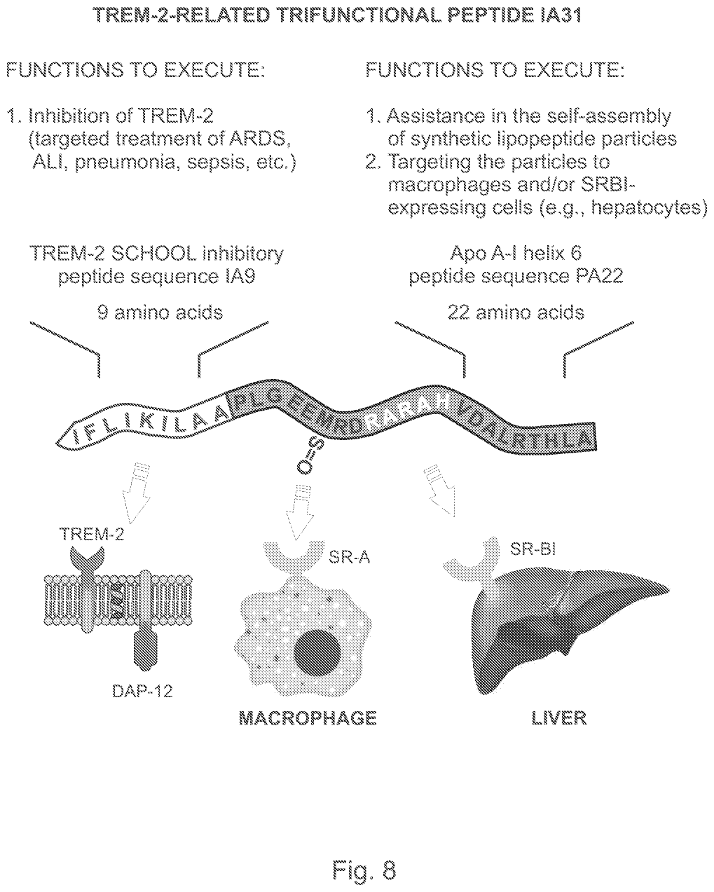

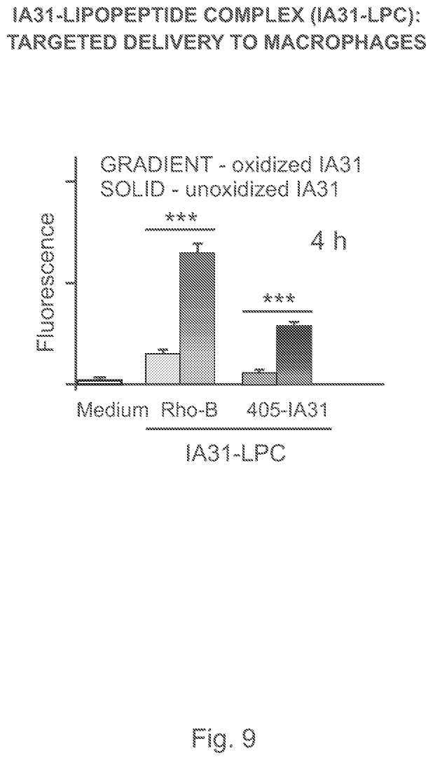

In certain embodiments, one domain of a trifunctional peptide comprises a TREM-2 inhibitor peptide sequence whereas the other domain comprises an unmodified or a modified apolipoprotein A-I helix 6 peptide sequence. Although it is not necessary to understand the mechanism of an invention, it is believed that in one embodiment, a TREM-2 inhibitor peptide sequence corresponding to a portion of a TREM-2 transmembrane domain sequence affects the TREM-2/DAP-12 receptor complex assembly (see FIG. 2) by inhibiting the TREM-2 signaling pathway and functions to treat and/or prevent a TREM-2-related disease or condition. For example, a 22 amino acids-long apolipoprotein A-I helix 6 peptide sequence with a sulfoxidized methionine residue functions to assist in the self-assembly of LPC upon binding to lipid or lipid mixtures and to target the particles to TREM-1-expressing cells (e.g., macrophages, microglia) and/or scavenger receptor BI (SRBI)-expressing cells (e.g., hepatocytes, cancer cells). In one embodiment, said TREM-2 inhibitor therapeutic peptide sequence comprises IFLIKILAA (SEQ. ID NO: 2). In one embodiment, said a 22 amino acids-long apolipoprotein A-I helix 6 peptide sequence with sulfoxidized methionine residue comprises PLGEEM(O)RDRARAHVDALRTHLA. In one embodiment, a trifunctional peptide comprises IFLIKILAAPLGEEM(O)RDRARAHVDALRTHLA (IA31) (SEQ. ID NO: 92). See FIG. 8. In one embodiment, an unmodified or a modified IA31 is complexed into LPC (IA31-LPC). In one embodiment, a modified IA31 targets IA31-LPC to macrophages. In one embodiment, said modified IA31 comprises an IA31 peptide with a sulfoxidized methionine residue. See FIGS. 8, 9.

In certain embodiments, one domain of a trifunctional peptide comprises a TREM-1 inhibitor peptide sequence whereas the other domain comprises an unmodified or a modified apolipoprotein A-I helix 6 peptide sequence. Although it is not necessary to understand the mechanism of an invention, it is believed that a TREM-1 inhibitor peptide sequence corresponding to a portion of a TREM-1 transmembrane domain sequence affects the TREM-1/DAP-12 receptor complex assembly (see FIG. 1) by inhibiting the TREM-1 signaling pathway and functions to treat and/or prevent a TREM-1-related disease or condition. For example, a 22 amino acids-long apolipoprotein A-I helix 6 peptide sequence with a sulfoxidized methionine residue functions to assist in the self-assembly of an LPC upon binding to lipid or lipid mixtures and to target the particles to TREM-1-expressing cells (eg, macrophages, neutrophils) and/or scavenger receptor BI (SRBI)-expressing cells (e.g., hepatocytes, cancer cells). In one embodiment, a TREM-1 inhibitor peptide sequence comprises GFLSKSLVF (GF9). In certain embodiments, TREM-1 inhibitory peptide sequence includes, but is not limited to, RGFFRGG (M3), LQEEDAGEYGCM (LR12) and LQVTDSGLYRCVIYHPP (LP17). In one embodiment, a 22 amino acids-long apolipoprotein A-I helix 6 peptide sequence with a sulfoxidized methionine residue comprises PLGEEM(O)RDRARAHVDALRTHLA. In one embodiment, a trifunctional peptide comprises GFLSKSLVFPLGEEM(O)RDRARAHVDALRTHLA (GA31). In one embodiment, an unmodified or a modified GA31 is complexed into LPC (GA31-LPC). In one embodiment, modified GA31 targets GA31-LPC to macrophages. In one embodiment, said modified GA31 comprises GA31 with sulfoxidized methionine residue.

In certain embodiments, one domain of a trifunctional peptide comprises a TREM-2 inhibitor peptide sequence whereas the other domain comprises an unmodified or a modified apolipoprotein A-I helix 4 peptide sequence. Although it is not necessary to understand the mechanism of an invention, it is believed that a TREM-2 inhibitor peptide sequence corresponding to a portion of a TREM-2 transmembrane domain sequence affects the TREM-2/DAP-12 receptor complex assembly (see FIG. 2) by inhibiting the TREM-2 signaling pathway and functions to treat and/or prevent a TREM-2-related disease or condition. For example, a 22 amino acids-long apolipoprotein A-I helix 4 peptide sequence with a sulfoxidized methionine residue functions to assist in the self-assembly of an LPC upon binding to lipid or lipid mixtures and to target the particles to TREM-1-expressing cells (eg, macrophages, microglia) and/or scavenger receptor BI (SRBI)-expressing cells (e.g., hepatocytes, cancer cells). In one embodiment, said TREM-2 inhibitory therapeutic peptide sequence comprises IFLIKILAA (SEQ. ID NO: 2). In one embodiment, said a 22 amino acids-long apolipoprotein A-I helix 4 peptide sequence with a sulfoxidized methionine residue comprises PYLDDFQKKWQEEM(O)ELRQKVE. In one embodiment, a trifunctional peptide comprises IFLIKILAAPYLDDFQKKWQEEM(O)ELRQKVE (IE31) (SEQ. ID NO: 93). In one embodiment, an unmodified or a modified IE31 is complexed into an LPC (IE31-LPC). In one embodiment, modified IE31 targets IE31-LPC to macrophages. In one embodiment, said modified IE31 comprises IE31 with sulfoxidized methionine residue.

In one embodiment, a TREM-1 inhibitor peptide sequence and 22 amino acids-long apolipoprotein A-I helix 4 peptide sequence with a sulfoxidized methionine residue can be used to assist in the self-assembly of LPC upon binding to lipid or lipid mixtures and to target the particles to TREM-1-expressing cells (eg, macrophages, neutrophils) and/or scavenger receptor BI (SRBI)-expressing cells (e.g., hepatocytes, cancer cells). In one embodiment, a TREM-1 inhibitor peptide sequence comprises GFLSKSLVF (GF9). In certain embodiments, TREM-1 inhibitory peptide sequence includes, but is not limited to, RGFFRGG (M3), LQEEDAGEYGCM (LR12) and LQVTDSGLYRCVIYHPP (LP17). In one embodiment, a 22 amino acids-long apolipoprotein A-I helix 4 peptide sequence with a sulfoxidized methionine residue comprises PYLDDFQKKWQEEM(O)ELRQKVE. In one embodiment, a trifunctional peptide comprises GFLSKSLVFPYLDDFQKKWQEEM(O)ELRQKVE. In one embodiment, an unmodified or a modified GE31 is complexed into an LPC (GE31-LPC). In one embodiment, a modified GE31 targets GE31-LPC to macrophages. In one embodiment, a modified GE31 comprises GE31 with sulfoxidized methionine residue.

In certain embodiments, one domain of a trifunctional peptide comprises a combinatorial TREM-1 and TREM-2 concurrent inhibitor peptide sequence whereas the other domain comprises an unmodified or a modified apolipoprotein A-I helix 6 peptide sequence. Although it is not necessary to understand the mechanism of an invention, it is believed that in one embodiment, a combinatorial TREM-1 and TREM-2 concurrent inhibitor peptide sequence comprising portions of TREM-1 and TREM-2 transmembrane domain sequences affects the TREM-1/DAP-12 and TREM-2/DAP-12 receptor complex assemblies (see FIGS. 1, 2) by inhibiting the TREM-1 and TREM-2 signaling pathways simultaneously and functions to treat and/or prevent a TREM-1 and/or TREM-2-related disease or condition. For example, a 22 amino acids-long apolipoprotein A-I helix 6 peptide sequence with a sulfoxidized methionine residue functions to assist in the self-assembly of LPC upon binding to lipid or lipid mixtures and to target the particles to TREM-1 and/or TREM-2-expressing cells (eg, macrophages, microglia, neutrophils) and/or scavenger receptor BI (SRBI)-expressing cells (e.g., hepatocytes, cancer cells). In one embodiment, a combinatorial TREM-1 and TREM-2 concurrent inhibitor peptide sequence comprises GFLSKSLVFIFLIKILAA (SEQ. ID NO: 49). In one embodiment, a 22 amino acids-long apolipoprotein A-I helix 6 peptide sequence with a sulfoxidized methionine residue comprises PLGEEM(O)RDRARAHVDALRTHLA. In one embodiment, a trifunctional peptide comprises GFLSKSLVFIFLIKILAAPLGEEM(O)RDRARAHVDALRTHLA (SEQ. ID NO: 98). In one embodiment, an unmodified or a modified GA40 is complexed into an LPC (GA40-LPC). In one embodiment, a modified GA40 targets GA40-LPC to macrophages. In one embodiment, a modified GA40 comprises GA40 with a sulfoxidized methionine residue.

In certain embodiments, one domain of a trifunctional peptide comprises a combinatorial concurrent inhibitor peptide sequence and/or combinations thereof, whereas the other domain comprises at least one modified or unmodified amphipathic apo A-I and/or A-II peptide fragment or combinations thereof. In one embodiment, exemplary combinatorial concurrent inhibitor peptide sequences are listed in TABLE 3. In one embodiment, a modified apo A-I peptide fragment comprises a 22 amino acids-long apolipoprotein A-I helix 6 peptide sequence with sulfoxidized methionine residue.

Exemplary embodiments of trifunctional peptide variants and compositions of the present invention are indicated in TABLE 5.

TABLE 5
Exemplary Trifunctional
Peptides and Compositions

| | SEQ |
|---|---|
| | ID |
| Amino Acid Sequence | NO: |
| IFLIKILAA, TREM-2 inhibitor | |
| IFLIKILAAPLGEEMRDRARAHVDALRTHLA | 90 |
| IFLIKILAAPYLDDFQKKWQEEMELYRQKVE | 91 |
| IFLIKILAAPLGEEM(O)RDRARAHVDALRTHLA | 92 |
| IFLIKILAAPYLDDFQKKWQEEM(O)ELRQKVE | 93 |
| IFLIKILAAPLGEEMRDRARAHVDALRTHLARGD | 94 |
| IFLIKILAAPYLDDFQKKWQEEMELYRQKVERGD | 95 |
| [64Cu]IFLIKILAAPLGEEM(O)RDRARAHVDALRTHLA | 96 |
| [64Cu]IFLIKILAAPYLDDFQKKWQEEM(O)ELRQKVE | 97 |
| GFLSKSLVFIFLIKILAA, TREM-1 and TREM-2 | |
| concurrent inhibitor | |
| GFLSKSLVFIFLIKILAAPLGEEMRDRARAHVDALRTHLA | 98 |
| GFLSKSLVFIFLIKILAAPYLDDFQKKWQEEMELYRQKVE | 99 |
| GFLSKSLVFIFLIKILAAPLGEEM(O)RDRARAHVDALRTHLA | 100 |
| GFLSKSLVFIFLIKILAAPYLDDFQKKWQEEM(O)ELRQKVE | 101 |
| GFLSKSLVFIFLIKILAAPLGEEMRDRARAHVDALRTHLARGD | 102 |
| GFLSKSLVFIFLIKILAAPYLDDFQKKWQEEMELYRQKVERGD | 103 |
| [64Cu]GFLSKSLVFIFLIKILAAPLGEEM(O)RDRARAHVDAL | 104 |
| RTHLA | |
| [64Cu]GFLSKSLVFIFLIKILAAPYLDDFQKKWQEEM(O)ELR | 105 |
| QKVE | |
| MWKTPTLKYFGFLSKSLVF, TCR and TREM-1 | |
| concurrent inhibitor | |
| MWKTPTLKYFGFLSKSLVFPLGEEMRDRARAHVDALRTHLA | 106 |
| MWKTPTLKYFGFLSKSLVFPYLDDFQKKWQEEMELYRQKVE | 107 |
| MWKTPTLKYFGFLSKSLVFPLGEEM(O)RDRARAHVDALRTHLA | 108 |
| MWKTPTLKYFGFLSKSLVFPYLDDFQKKWQEEM(O)ELRQKVE | 109 |
| MWKTPTLKYFGFLSKSLVFPLGEEMRDRARAHVDALRTHLARGD | 110 |
| MWKTPTLKYFGFLSKSLVFPYLDDFQKKWQEEMELYRQKVERGD | 111 |
| [64Cu]MWKTPTLKYFGFLSKSLVFPLGEEM(O)RDRARAHVDA | 112 |
| LRTHLA | |
| [64Cu]MWKTPTLKYFGFLSKSLVFPYLDDFQKKWQEEM(O)EL | 113 |
| RQKVE | |
| MWKTPTLKYFIFLIKILAA, TCR and TREM-2 | |
| concurrent inhibitor | |
| MWKTPTLKYFIFLIKILAAPLGEEMRDRARAHVDALRTHLA | 114 |
| MWKTPTLKYFIFLIKILAAPYLDDFQKKWQEEMELYRQKVE | 115 |
| MWKTPTLKYFIFLIKILAAPLGEEM(O)RDRARAHVDALRTHLA | 116 |
| MWKTPTLKYFIFLIKILAAPYLDDFQKKWQEEM(O)ELRQKVE | 117 |
| MWKTPTLKYFIFLIKILAAPLGEEMRDRARAHVDALRTHLARGD | 118 |
| MWKTPTLKYFIFLIKILAAPYLDDFQKKWQEEMELYRQKVERGD | 119 |
| [64Cu]MWKTPTLKYFIFLIKILAAPLGEEM(O)RDRARAHVDA | 120 |
| LRTHLA | |
| [64Cu]MWKTPTLKYFIFLIKILAAPYLDDFQKKWQEEM(O)EL | 121 |
| RQKVE | |

### III. Therapeutic Applications of Peptide Variants and Compositions of the Invention

In certain embodiments, preferred peptide variants and compositions as described herein can be used in combination with other TREM-1 inhibitory peptide sequences such as RGFFRGG (M3), LQEEDAGEYGCM (LR12) and LQVTDSGLYRCVIYHPP (LP17) as described in (Gibot et al. 2006b, Gibot et al. 2007, Gibot et al. 2009, Zhou et al. 2013, Joffre et al. 2016, Cuvier et al. 2018, Denning et al. 2020a, Denning et al. 2020b, Denning et al. 2020c, Siskind et al. 2022) and disclosed in U.S. Pat. Nos. 8,013,116; 9,273,111; 9,657,081; 9,815,883; 10,603,357; 9,255,136 (all of which are herein incorporated by reference).

In certain embodiments, preferred peptide variants and compositions as described herein can be used in combination with anti-TREM-1 and/or anti-TREM-2 antibodies as described in (Brynjolfsson et al. 2016, Molgora et al. 2020, Binnewies et al. 2021) and disclosed in U.S. Pat. Nos. 11,186,636; 11,155,618; 11,124,567; and 10,508,148 (all of which are herein incorporated by reference); and WO 2017/0152102.

### 1. Sepsis, Cytokine Storm, ARDS and Other Lung Injuries and Diseases

### a) Sepsis and Cytokine Storm

Sepsis is a life-threatening condition that occurs when the body's response to an infection damages its own tissues. No sepsis drugs are available and over thirty (30) drugs failed in late-stage clinical trials (Marshall 2014), including: i) TNFa and IL-1 blockers (Remick 2003); ii) human activated protein C (Annane et al. 2013); and iii) TLR4 antagonist (Opal et al. 2013). This highlights an urgent need for new therapies.

Sepsis is associated with an overwhelming production of proinflammatory cytokines (the so-called “cytokine storm”) produced mainly by macrophages (Cavaillon et al. 2003, Riedemann et al. 2003, Peck et al. 2009). In sepsis, TREM-1 expression is increased on monocytes and macrophages (Gibot 2005, Ferat-Osorio et al. 2008, van Bremen et al. 2013). TREM-1 activation induces MCSF (or CSF-1) (Dower et al. 2008). Activation, growth and differentiation of macrophages are regulated by CSF-1 which is overproduced in septic patients (Francois et al. 1997). In septic patients, high TNFa, IL-1, and IL-6 have been correlated with a poor clinical outcome (Gogos et al. 2000, Oberholzer et al. 2005).

Blockade of TREM-1 lowers TNFa, Il-1b, CSF-1, and IL-6 and promotes survival in septic mice as described herein (see also, (Bouchon et al. 2001, Gibot et al. 2007, Sigalov 2014b) and in mice with Pseudomonas aeruginosa-induced peritonitis but has no effect on in vitro macrophage phagocytosis (Wang et al. 2012). In pigs and primates, administration of LR12, an antagonistic peptide that likely blocks binding of an unknown ligand to TREM-1, mitigates endotoxin-associated clinical and biological alterations, with no obvious side effects (Derive et al. 2013, Derive et al. 2014).

In experimental sepsis, data on the effect of blockade or deficiency of TREM-2 are controversial. Some studies indicate that blockade of TREM-2 results in markedly increased mortality (Chen et al. 2013, Gawish et al. 2015). In contrast, other studies suggest that TREM-2 deficiency restricts the inflammatory response, thereby decreasing organ damage and mortality (Weehuizen et al. 2016).

In one embodiment, the present invention contemplates peptide variants and compositions and methods to provide an effective and well-tolerated therapy for treating sepsis and cytokine storm aimed to reduce the mortality rate and improve outcomes.

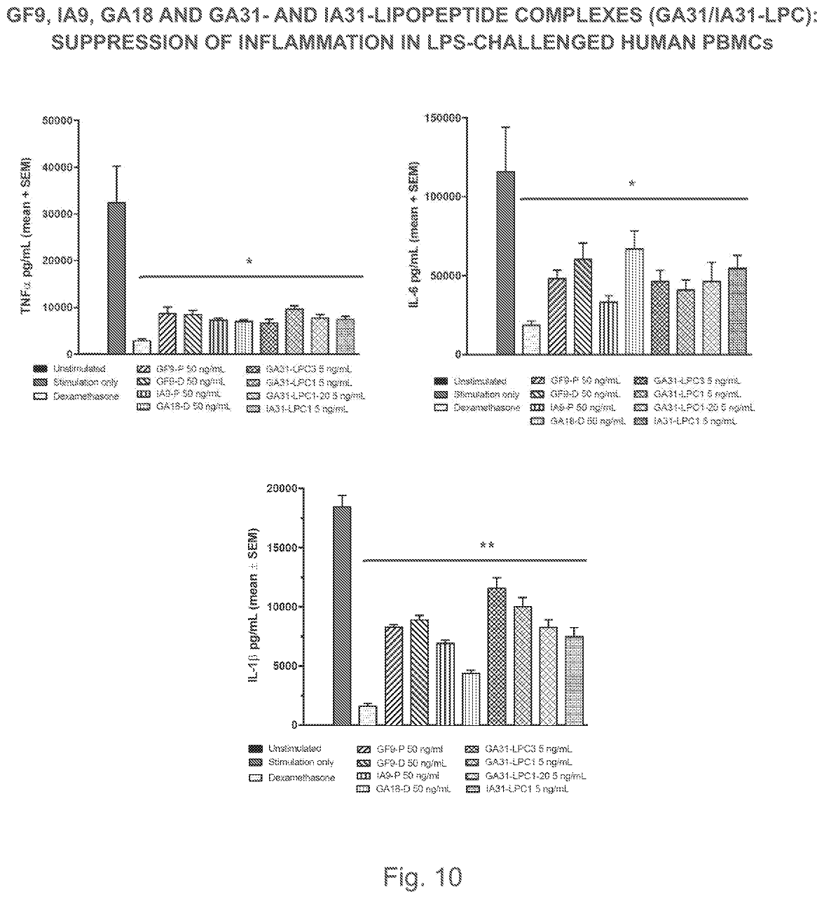

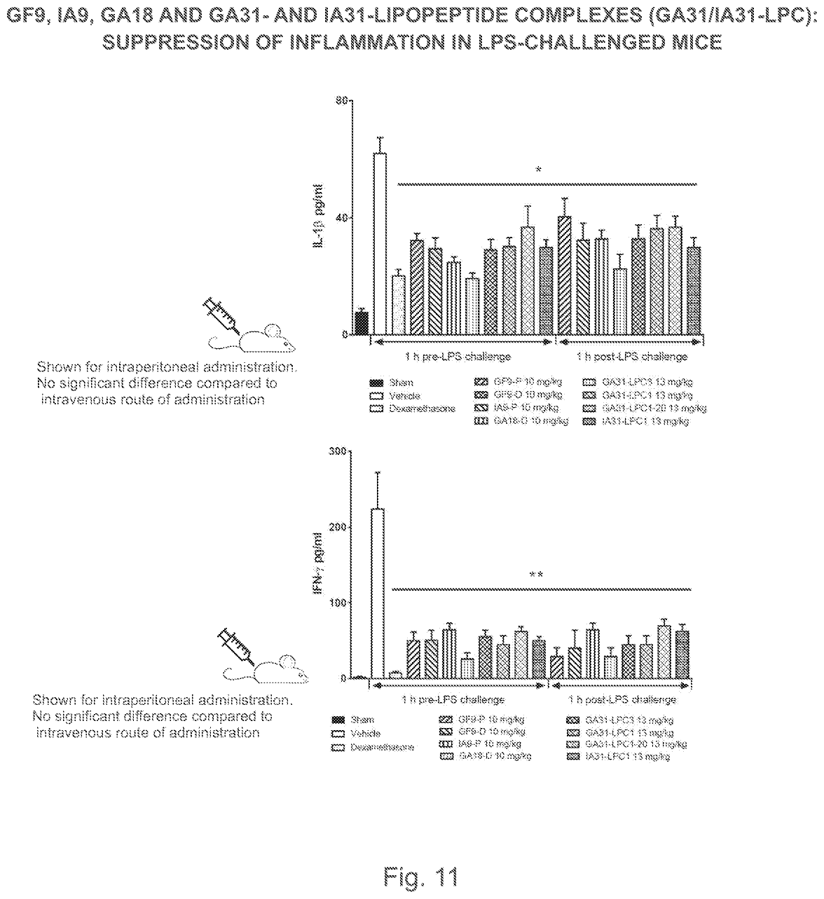

In certain embodiments, free peptides such as GF9, IA9 (SEQ. ID NO: 2) and/or GA18 (SEQ. ID. NO: 49) at 50 ng/mL significantly reduce release of proinflammatory cytokines by human peripheral blood mononuclear cell (PBMC) stimulated with lipopolysaccharide (LPS). See FIG. 10.

In some embodiments, water-insoluble peptides GF9 and IA9 are solubilized in a mixture of propylene glycol, ethanol and Tween-80 (GF9-P and IA9-P). In some embodiments, water-insoluble peptides GF9 and GA18 are formulated in a pharmacologically acceptable excipient, sulfobutylether-beta-cyclodextrin (SBECD). In one embodiment, SBECD represents Dexolve, a generic form of Captisol (GF9-D and GA18-D). In one embodiment, in contrast to GF9 and GA-18, IA9 is not soluble in SBECD-based formulations. In certain embodiments, combinatorial TREM-1 and TREM-2 concurrent inhibitor peptide GA18 is more effective in suppressing cytokine release compared to that of either TREM-1 inhibitor peptide GF9 or TREM-2 inhibitor peptide IA9. See FIG. 10.

In some embodiments, a TREM-1 inhibitor peptide sequence (e.g., GF9) is used as a part of trifunctional peptide GA31. In some embodiments, a TREM-2 inhibitor peptide sequence (e.g., IA9) is used as a part of a trifunctional peptide IA31 (SEQ. ID NO: 92). In certain embodiments, GA31 and IA31 are formulated in targeted LPCs (GA31-LPC and IA31-LPC, respectively). In one embodiment, GA31-LPC comprises a complex of GA31 with three lipid components (GA31-LPC3): for example, 1-palmitoyl-2-oleoyl-sn-glycero-3-phosphocholine (POPC), cholesterol and cholesteryl oleate. In one embodiment. GA31-LPC comprises a complex of GA31 with one lipid component. In one embodiment, this lipid component comprises POPC (GA31-LPC1). In one embodiment, GA31-LPC1 comprises GA31-LPC1 with decreased POPC-GA31 molar ratio (GA31-LPC1-20). In one embodiment, IA31-LPC comprises a complex of IA31 with one lipid component. In one embodiment, this lipid component comprises POPC (IA31-LPC1). In certain embodiments, a GA31-LPC3 complex, a GA31-LPC1 complex, a GA31-LPC1-20 complex and/or a IA31-LPC complex significantly reduce release of proinflammatory cytokines by LPS-challenged human PBMCs. See FIG. 10.

In certain embodiments, a free peptide formulation of GF9-P, GF9-D, IA9-P and/or GA18-D as described herein significantly suppress release of plasma proinflammatory cytokines in mice challenged by LPS, when preventatively (1 hour before LPS challenge), or therapeutically (1 hour post-LPS challenge), administered intraperitoneally (i.p.) or intravenously (i.v.) at 10 mg/kg dose. In certain embodiments, targeted LPC-based formulations including, but not limited to, GA31-LPC3, GA31-LPC1, GA31-LPC1-20 or IA31-LPC as described herein significantly suppress release of plasma proinflammatory cytokines in mice challenged by LPS, when preventatively (1 hour before LPS challenge), or therapeutically (1 hour post-LPS challenge), administered i.p. or i.v. at 13 mg of the corresponding peptide/kg dose. In one embodiment, GA18 is more effective compared to GF9 or IA9. In one embodiment, no significant differences in suppressing cytokine release are observed for the formulations described herein administered either i.p. or i.v. See FIG. 11.

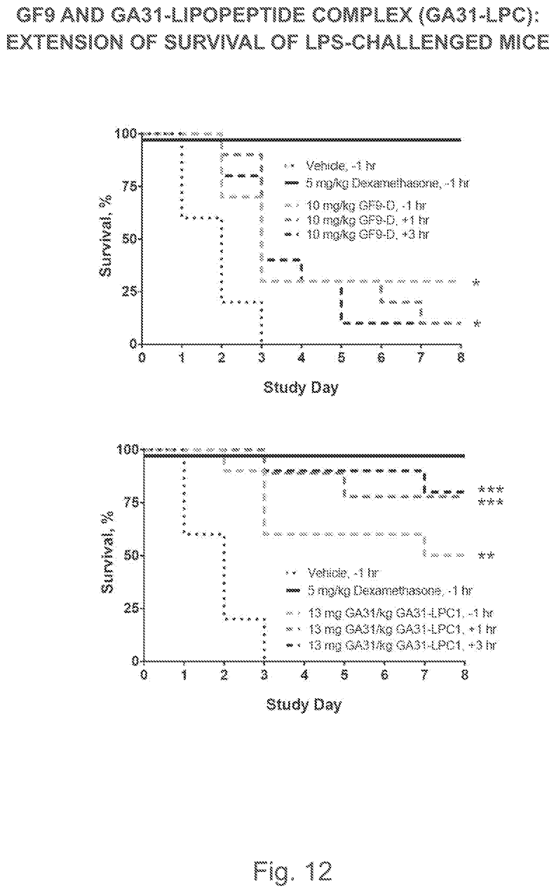

In certain embodiments, free peptide- and LPC-based TREM-1 inhibitory formulations GF9-D and GA31-LPC1 as described herein significantly extend survival of mice challenged by LPS, when preventatively (1 hour before LPS challenge), or therapeutically (1 or 3 hour post-LPS challenge), administered i.p. at 10 mg/kg (GF9-D) and 13 mg/kg (GA31-LPC1) doses. In one embodiment, GA31-LPC1 provides more effective and longer-lasting protection compared with that of GF9. These data support the hypothesized longer half-life of the peptide when formulated into an HDL-mimicking LPC. In one embodiment, survival extension of mice treated with GF9-D correlates with time of administration. In contrast, in one embodiment, survival extension of mice treated with GA31-LPC1 inversely correlates with time of administration. While not being bound to any particular theory, it is believed that this phenomenon can be explained by different effects of pan-TREM-1 inhibitor GF9-D and targeted TREM-1 inhibitor GA31-LPC1 on systemic and local inflammation in the pathogenesis of sepsis. See FIG. 12.

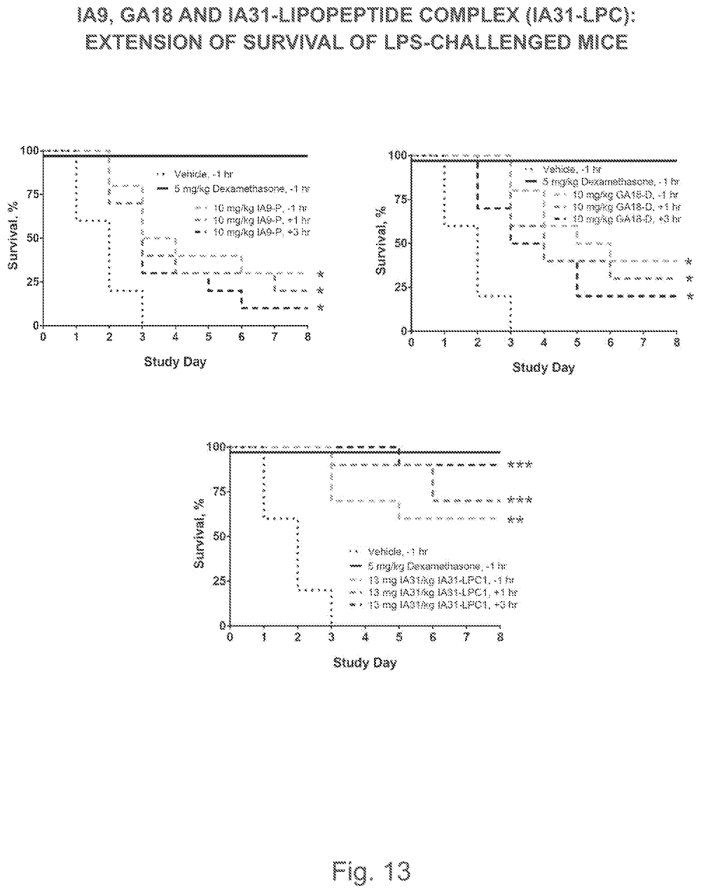

In certain embodiments, free peptide- and LPC-based TREM-2 inhibitory formulations IA9-P, GA18-D and IA31-LPC1 as described herein significantly extend survival of mice challenged by LPS, when preventatively (1 hour before LPS challenge), or therapeutically (1 or 3 hour post-LPS challenge), administered i.p. at 10 mg/kg (IA9-P and GA18-D) and 13 mg/kg (IA31-LPC1) doses. In one embodiment, IA31-LPC1 provides more effective and longer-lasting protection compared with that of IA9 and GA18. These data support the hypothesized longer half-life of the peptide formulated into HDL-mimicking LPC. In one embodiment, survival extension of mice treated with IA9-P and GA18-D correlates with time of administration. In contrast, in one embodiment, survival extension of mice treated with IA31-LPC1 inversely correlates with time of administration. While not being bound to any particular theory, it is believed that this phenomenon can be explained by different effects of pan-TREM-2 inhibitors IA9-P and GA18-D and targeted TREM-2 inhibitor IA31-LPC1 on systemic and local inflammation in the pathogenesis of sepsis. See FIG. 13.

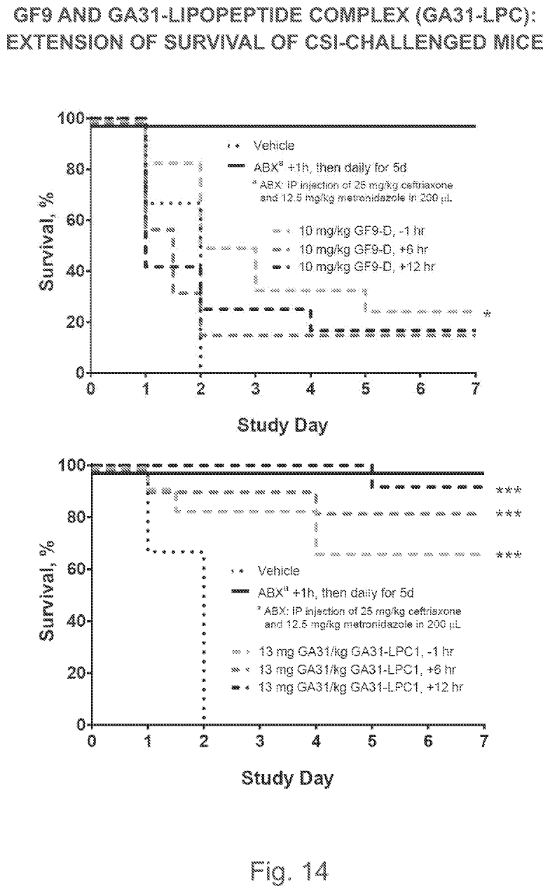

A reported cecal slurry (CS) injection sepsis model has been reported, in which the cecal contents of a laboratory animal are injected intraperitoneally to other animals, and is an art accepted model for induced polymicrobial sepsis (Starr et al. 2014). In certain embodiments, free peptide- and LPC-based TREM-1 inhibitory formulations GF9-D and GA31-LPC1 as described herein significantly extend survival of mice challenged by CS, when preventatively (1 hour before CS challenge), or therapeutically (6 or 12 hours post-CS challenge), administered i.p. at 10 mg/kg (GF9-D) and 13 mg/kg (GA31-LPC1) doses. In one embodiment, GA31-LPC1 provides more effective and longer-lasting protection compared with that of GF9. These data support the hypothesized longer half-life of the peptide formulated into HDL-mimicking LPC. In one embodiment, survival extension of mice treated with GF9-D correlates with time of administration. In contrast, in one embodiment, survival extension of mice treated with GA31-LPC1 inversely correlates with time of administration. While not being bound to any particular theory, it is believed that this phenomenon can be explained by different effects of pan-TREM-1 inhibitor GF9-D and targeted TREM-1 inhibitor GA31-LPC1 on systemic and local inflammation in the pathogenesis of sepsis. See FIG. 14.

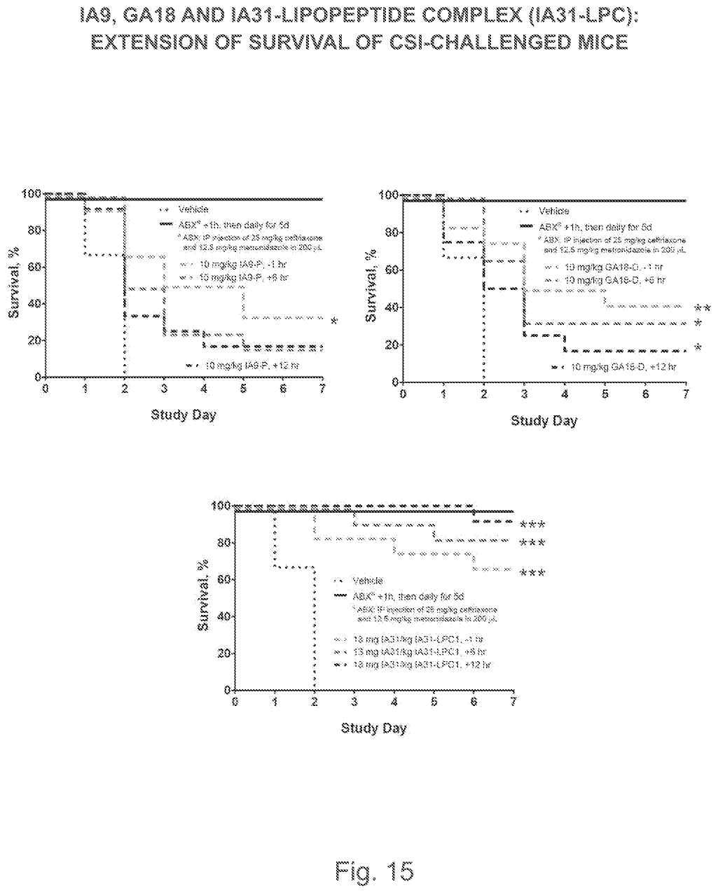

In certain embodiments, free peptide- and LPC-based TREM-2 inhibitory formulations IA9-P, GA18-D and/or IA31-LPC1 as described herein significantly extend survival of mice challenged by CS, when preventatively (1 hour before CS challenge), or therapeutically (6 or 12 hours post-CS challenge), administered i.p. at 10 mg/kg (IA9-P and GA18-D) and 13 mg/kg (IA31-LPC1) doses. In one embodiment, IA31-LPC1 provides more effective and longer-lasting protection as compared with that of IA9 and GA18. These data support the hypothesized longer half-life of the peptide formulated into HDL-mimicking LPC. In one embodiment, survival extension of mice treated with IA9-P and GA18-D correlates with time of administration. In contrast, in one embodiment, survival extension of mice treated with IA31-LPC1 inversely correlates with time of administration. While not being bound to any particular theory, it is believed that this phenomenon can be explained by different effects of pan-TREM-2 inhibitors IA9-P and GA18-D and targeted TREM-2 inhibitor IA31-LPC1 on systemic and local inflammation in the pathogenesis of sepsis. See FIG. 15.

### b) ARDS and Other Lung Injuries, Diseases and Conditions

ARDS is a common cause of respiratory failure in ˜10% of all critically ill patients in intensive care units (ICU) worldwide with mortality remaining high at 30-40% (Matthay et al. 2019). See, U.S. Pat. Nos. 9,205,100; 10,143,709; US 2018/0185372; US 2016/0215284; and US 2020/0023002 (all of which are herein incorporated by reference). Along with sepsis, ARDS is a common cause of death related to COVID-19 (Matthay et al. 2020). Current treatment of ARDS focuses on lung-protective ventilation because no specific therapeutic drugs are available (Standiford et al. 2016).

Pathogenesis of ARDS results in exhibiting symptoms that include, but are not limited to, inflammation of the lung parenchyma, infiltration of neutrophils into the airspaces, oxidative stress, disruption of the endothelial and epithelial barriers, damage to the epithelial lining and subsequent lung fibrosis. Despite the fact that the mechanisms contributing to the pulmonary failure are well delineated, more than 20 years of clinical trials show that approaches aiming at the separate components of pathogenesis fail to improve mortality. As of now, ARDS treatment remains primarily supportive and consists of the patient oxygenation with the lung protective ventilation strategy, and the tight control over the patient's fluid balance.

The known failures to alleviate ARDS with numerous pharmacological and non-pharmacological strategies has generated a long felt need for an alternative therapy which would not only reduce the pathogenic mechanisms of ARDS but also facilitate lung repair.

Due to the significant morbidity and mortality associated with ARDS and the lack of effective treatment options, new therapeutic agents for the treatment of ARDS and new treatment methods for ARDS are needed.

The present disclosure addresses the unmet need in the art by providing novel targeted therapeutic agents useful in the treatment of ARDS and methods of treatment for ARDS and conditions related thereto through the administration of such novel therapeutic agents.

In one embodiment, the present invention contemplates cell surface receptor inhibitor peptide variants, compositions and methods to provide an effective and well-tolerated COVID-19 infection therapy aimed to reduce the mortality rate and improve outcomes. COVID-19 infections are believed to induce an amplified, rapid acute inflammatory response in the lungs associated with a cytokine storm. In certain embodiments, said cell surface receptor inhibitor peptide variants are TREM-1 and/or TREM-2 inhibitor peptide variants and combinatorial TREM-1 and TREM-2 concurrent inhibitor peptide variants. As described herein, and as previously reported TREM-1 and/or TREM-2 inhibitor peptide variants and compositions exert both anti-inflammatory and pro-angiogenic activity. (Sigalov 2014b, Shen and Sigalov 2017b, Shen and Sigalov 2017a, Rojas et al. 2018, Tornai et al. 2019, Sigalov 2020, Gallop et al. 2021),

In certain embodiments, the present invention contemplates compositions and methods for a combination therapy comprising cell surface receptor inhibitor peptide variants and a therapeutic drug for treating and/or preventing inflammation and/or graft rejection associated with organ transplantation, in particular lung transplantation, including treatment, prevention or attenuation of the progression of conditions including, but not limited to, primary graft failure, ischemia-reperfusion injury, reperfusion injury, reperfusion edema, allograft dysfunction, pulmonary reimplantation response, bronchiolitis obliterans after lung transplantation and/or PGD after organ transplantation, in particular in lung transplantation.

ARDS develops most commonly in the setting of pneumonia (bacterial and viral; fungal is less common), nonpulmonary sepsis (with sources that include the peritoneum, urinary tract, soft tissue and skin), aspiration of gastric and/or oral and oesophageal contents (which may be complicated by subsequent infection) and major trauma (such as blunt or penetrating injuries or burns). In certain embodiments, said cell surface receptor inhibitor peptide variants are TREM-1 and/or TREM-2 inhibitor peptide variants and combinatorial TREM-1 and TREM-2 concurrent inhibitor peptide variants.

Other less common etiologies are also associated with the development of ARDS, including acute pancreatitis; transfusion of fresh frozen plasma, red blood cells and/or platelets (that is, transfusion-associated acute lung injury (TRALI)); drug overdose with various agents; near drowning (inhalation of fresh or salt water); haemorrhagic shock or reperfusion injury (including after cardiopulmonary bypass and lung resection); and smoke inhalation (often associated with cutaneous burn injuries).

Non-cardiogenic pulmonary oedema is also a cause of ARDS and includes, but is not limited to, primary graft dysfunction following lung transplantation, high-altitude pulmonary oedema, neurogenic oedema (following a central nervous system insult or injury) and drug-induced lung injury.

In some aspects and embodiments, provided are compositions and methods for mono- and/or combination therapy for treating lung disorders or injury in a mammal.

In some embodiments, the compounds or compositions to treat ARDS are selected peptide variants and compositions that modulate the TREM-1/DAP-12 signaling pathway including but not limited to those described herein and disclosed in U.S. Pat. Nos. 9,981,004; 8,513,185; 9,815,883; 9,273,111; and 8,013,116 (all of which are herein incorporated by reference); and PCT/US2010/052117; PCT/US2019/046392; and WO 2020/0369987.

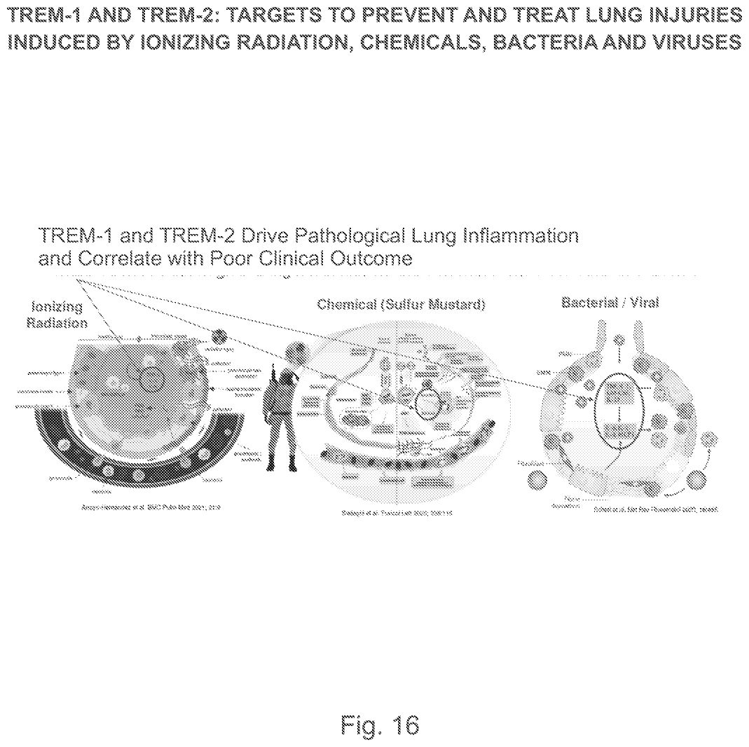

While not being bound to any particular theory, it is believed that TREM-1 and TREM-2 are involved in the pathogenesis of pathological inflammation in ARDS and other lung injuries and diseases caused by seemingly unrelated agents and conditions such as radiation (including but not limited to ionizing radiation), chemicals (including but not limited to phosgene, chlorine, sulfur mustard, etc.), bacteria (e.g., Bordetella pertussis, Pseudomonas aeruginosa, Streptococcus pneumoniae, Staphylococcus aureus, Legionella pneumophila, Pneumocystis jirovecii, etc.) and various respiratory viruses (SARS-CoV-1; SARS-CoV-2; avian influenza virus H5N1; adenovirus, ADV; human bocavirus, HBoV; human coronavirus, HCoV; human metapneumovirus, HMPV; human parainfluenza virus, HPIV; human rhinovirus HRV; human respiratory syncytial virus, HRSV, Herpesviridae, etc.) and enteric gram-negative organisms. Recently, similarity in inflammatory response (ARDS) of lung to radiation- and COVID-19-induced injury has been reported (Lazzari et al. 2020). This implies TREM-1 and TREM-2 as promising targets for these and other lung injuries and diseases where lung inflammation is involved. See FIG. 16.

### i) Ionizing Radiation-Induced Lung Injuries

Ionizing Radiation (IR) induces an immune response and inflammation not only in irradiated but also in non-irradiated sites in vivo. IR-induced lung injury encompasses two phases: an early phase known as radiation pneumonitis, characterized by acute lung tissue inflammation including ARDS as a result of exposure to radiation; and a late phase called radiation fibrosis, a clinical syndrome that results from chronic pulmonary tissue damage (Arroyo-Hernandez et al. 2021). Macrophages are recruited at these sites and upon activation, produce bystander signals and play an important role in the development of radiation injury. One example is a mobilization of inflammatory cells into irradiated sites in the lung affected by radiation during the acute phase. Activated macrophages release pro-inflammatory cytokines and CSF-1, the factor that regulates macrophage recruitment, activation, growth and differentiation (Elgert et al. 1998, Varney et al. 2005). Macrophages are a new therapeutic target in radiation injury (Meziani et al. 2018a). Blockade of CSF-1 receptor prevents radiation pulmonary fibrosis by depletion of interstitial macrophages (Meziani et al. 2018b).

Survivability of radiation casualties is determined by a progression of systemic inflammatory response syndrome (SIRS) to multiple organ dysfunction syndrome (MODS) thought to be mediated by the release of pro-inflammatory cytokines (Macia et al. 2011). This progression to MODS usually represents the final stages in a continuum of events associated with uncontrolled inflammatory responses and loss of vascular homeostasis, resulting in multiple organ failure (MOF), which is strongly linked to systemic inflammation (Jackson et al. 2005, Schmid-Schonbein 2006, Williams et al. 2011).

In certain embodiments, the present invention contemplates peptide variants, compositions and a method comprising protecting and treating an individual from acute radiation sickness (ARS) by a pre- or post-exposure administration of the peptide variants and compositions of the invention as disclosed herein. In one embodiment, the peptide variants and compositions of the invention are the TREM-1 and/or TREM-2 inhibitor peptides including combinatorial TREM-1 and TREM-2 concurrent inhibitor peptides and compositions of the invention. In one embodiment, the TREM-1 and or TREM-2 inhibitor radioprotective agents of the invention prevent and treat acute (including but not limited to ARDS) and/or late (including but not limited to lung fibrosis) radiation effects. In one embodiment, the TREM-1 and/or TREM-2 inhibitor radioprotective agents of the invention significantly extend an individual's survival as compared to conventional radiation exposure treatment therapies.

### ii) Bacteria-Induced Lung Injuries

Affectations of the lung (pneumonia, aspiration, and pulmonary contusion) cause direct ARDS, extrapulmonary (systemic) diseases (non-pulmonary sepsis, non-thoracic trauma, and transfusion) indirect ARDS. The majority of ARDS cases are caused by severe pneumonia (30-50%), sepsis (25-30%), and severe trauma 10-25% (Frohlich 2021). Bacteria-induced ARDS (Streptococcus pneumonia, Staphylococcus aureus, etc.) is more frequent than viral-induced ARDS (influenza A) or fungal ARDS (Pneumocystis jirovecii).

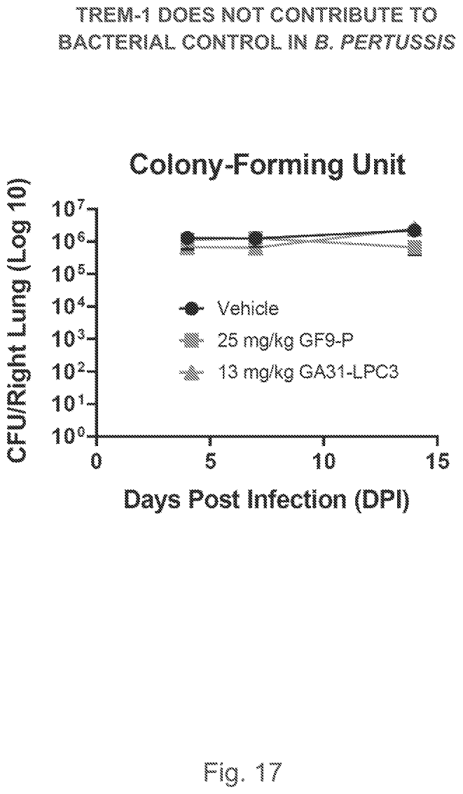

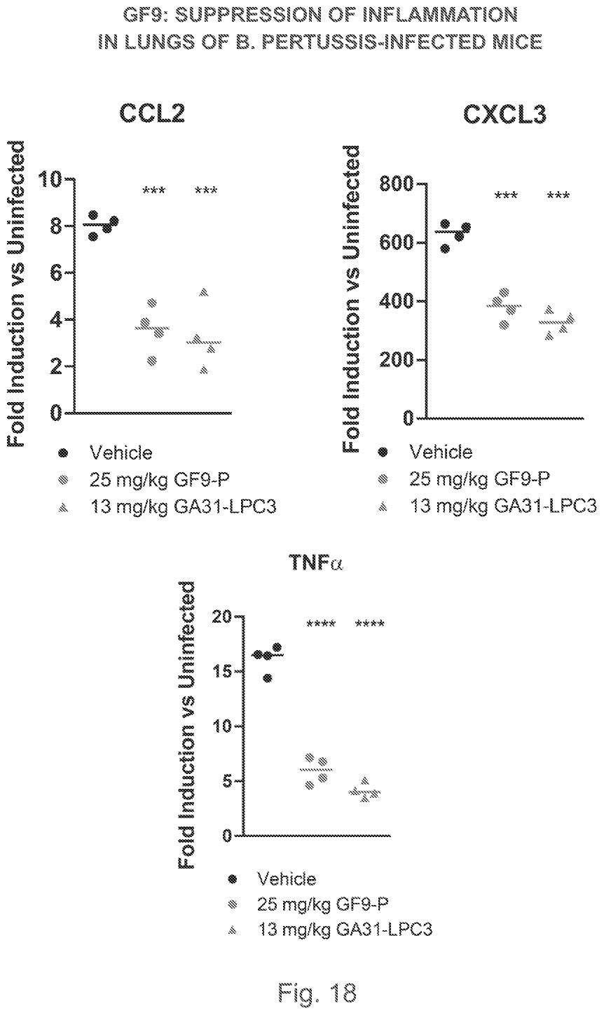

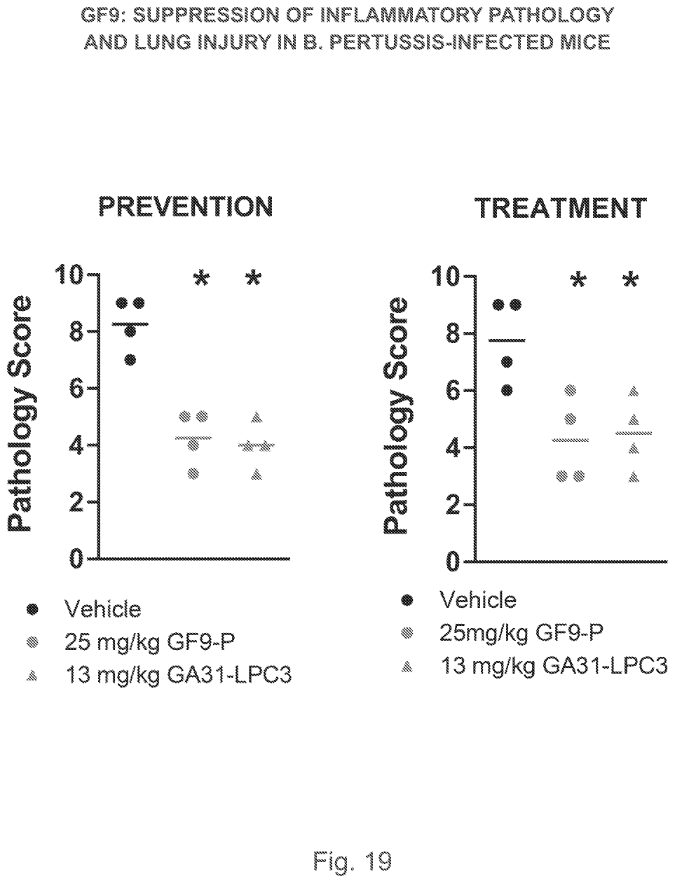

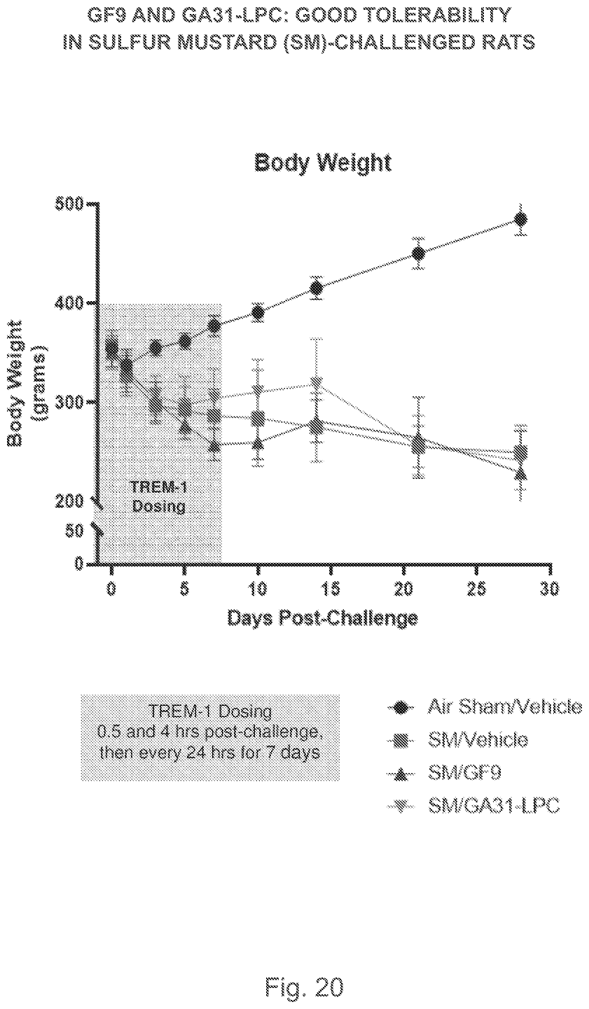

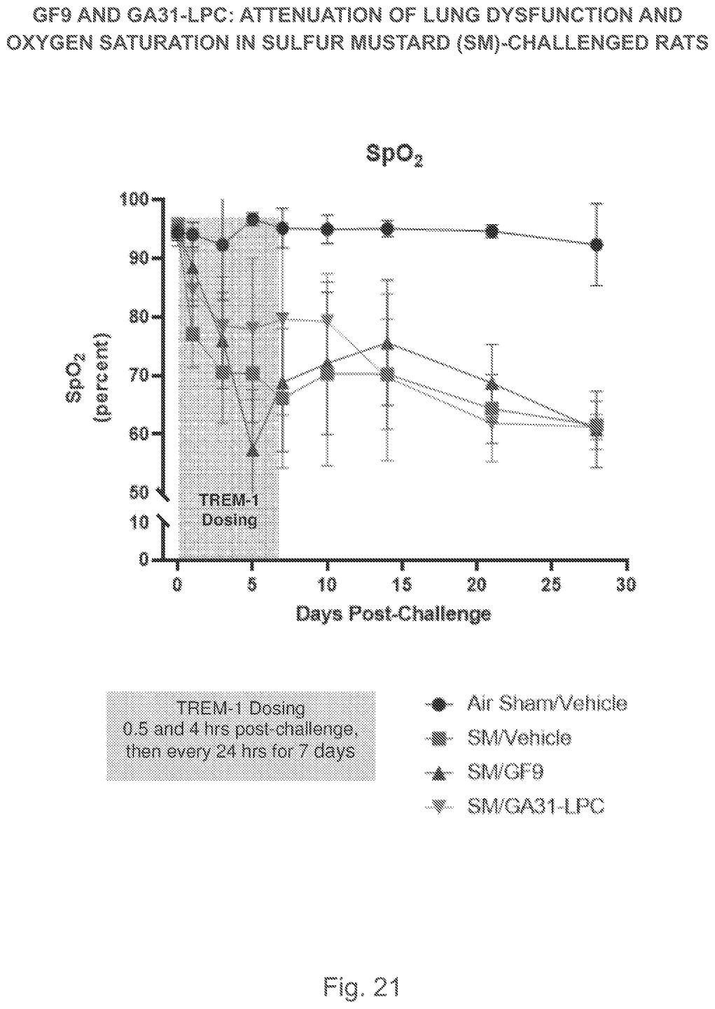

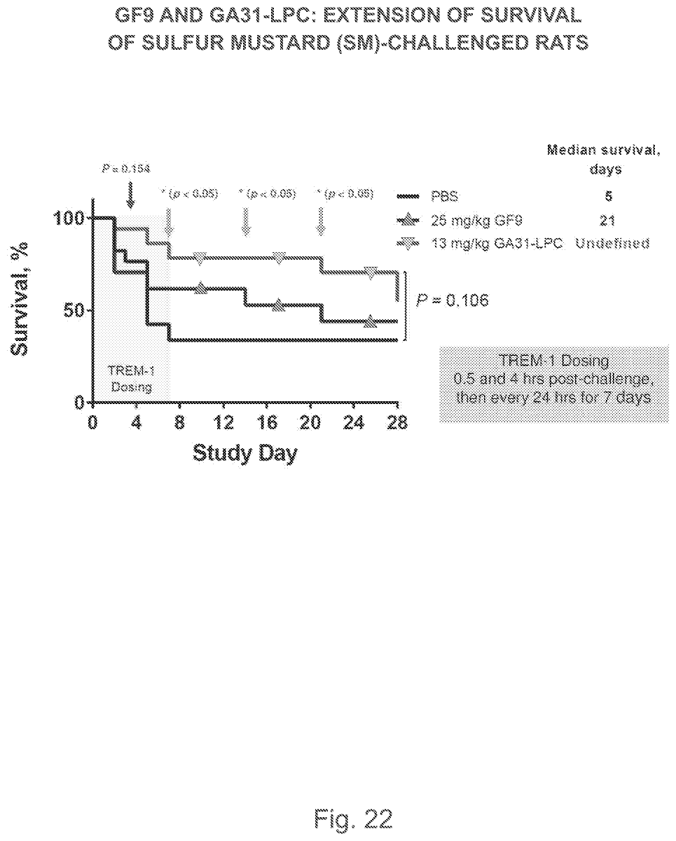

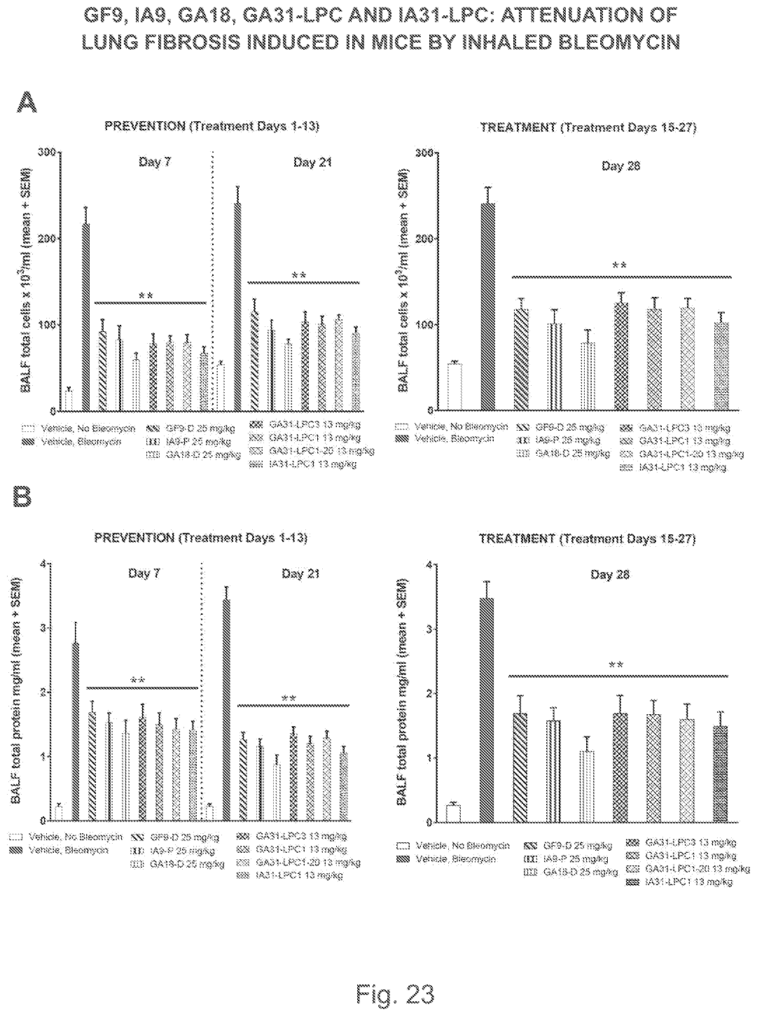

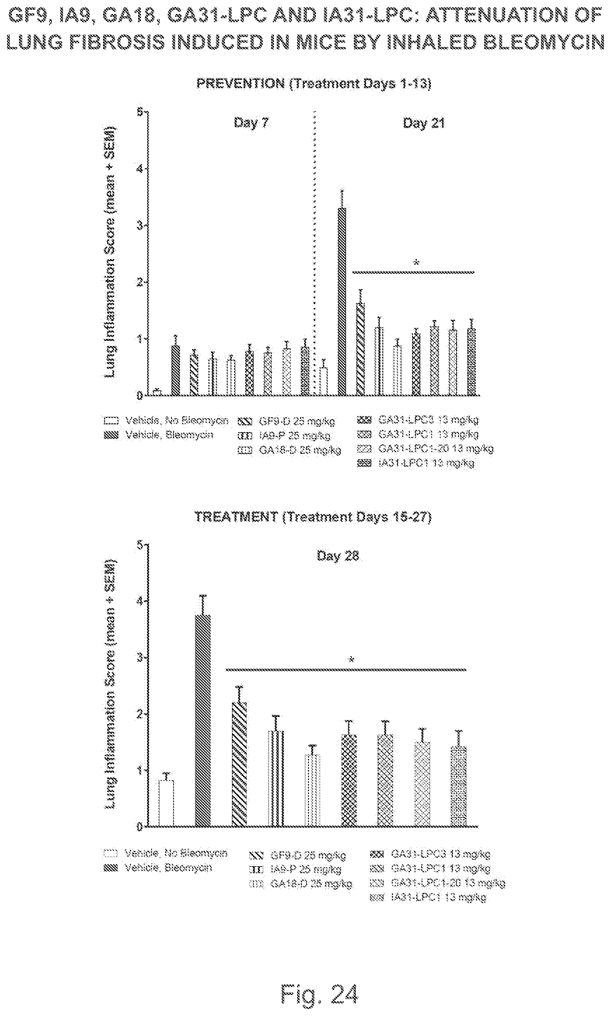

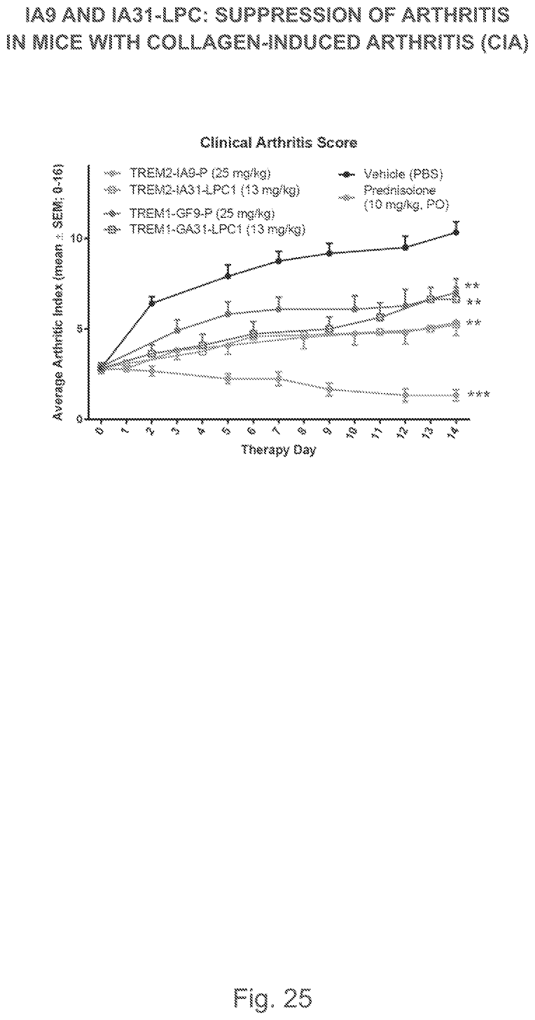

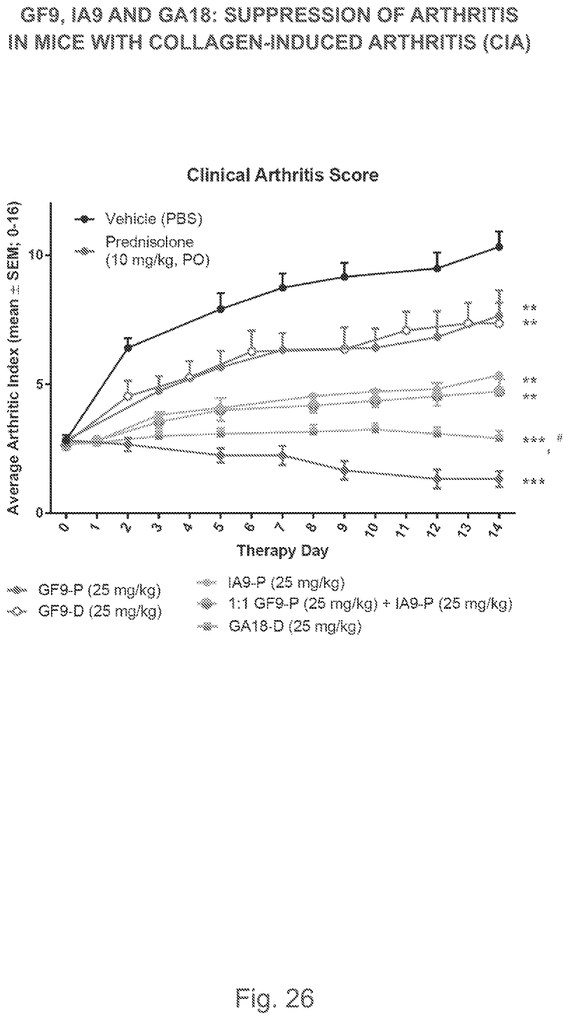

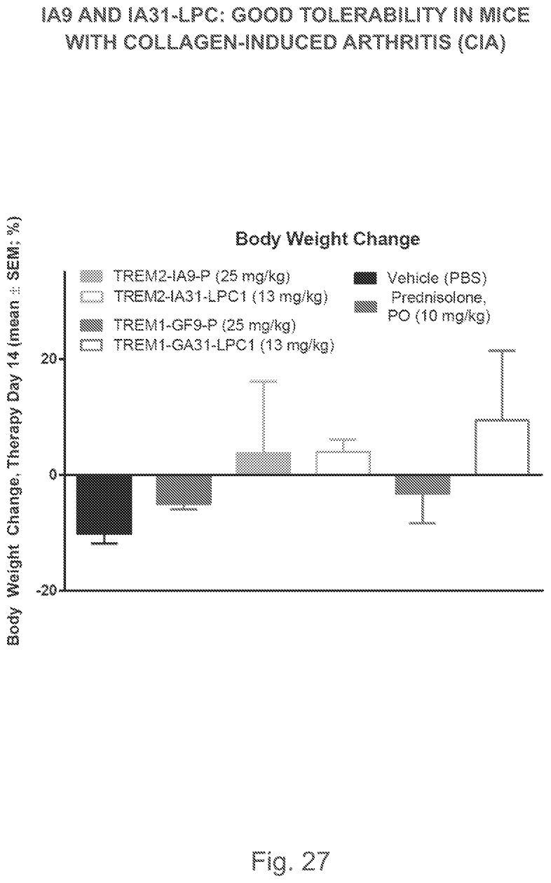

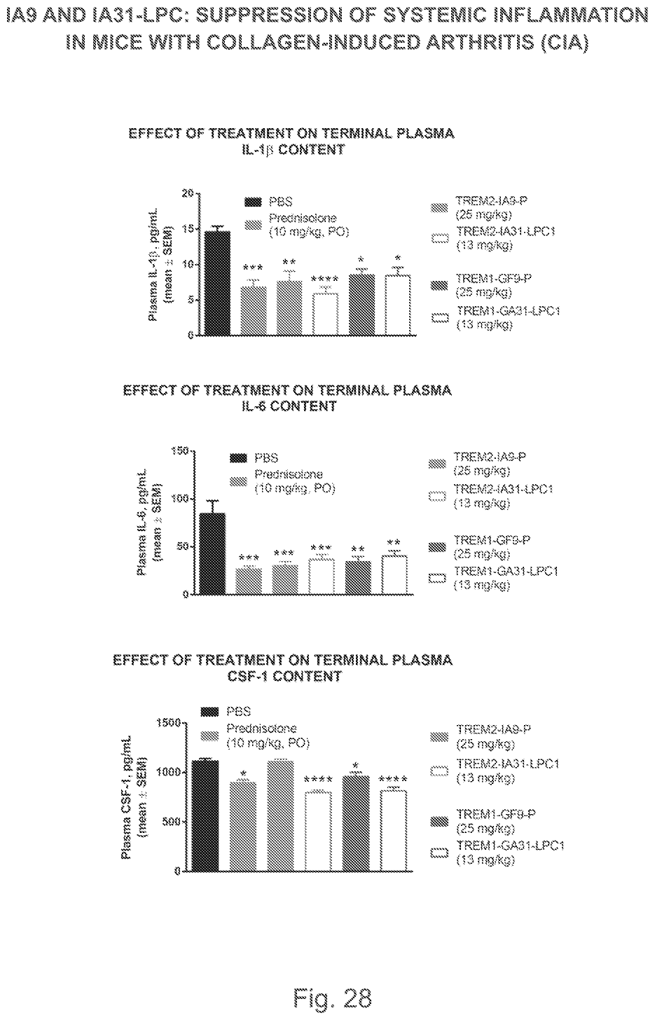

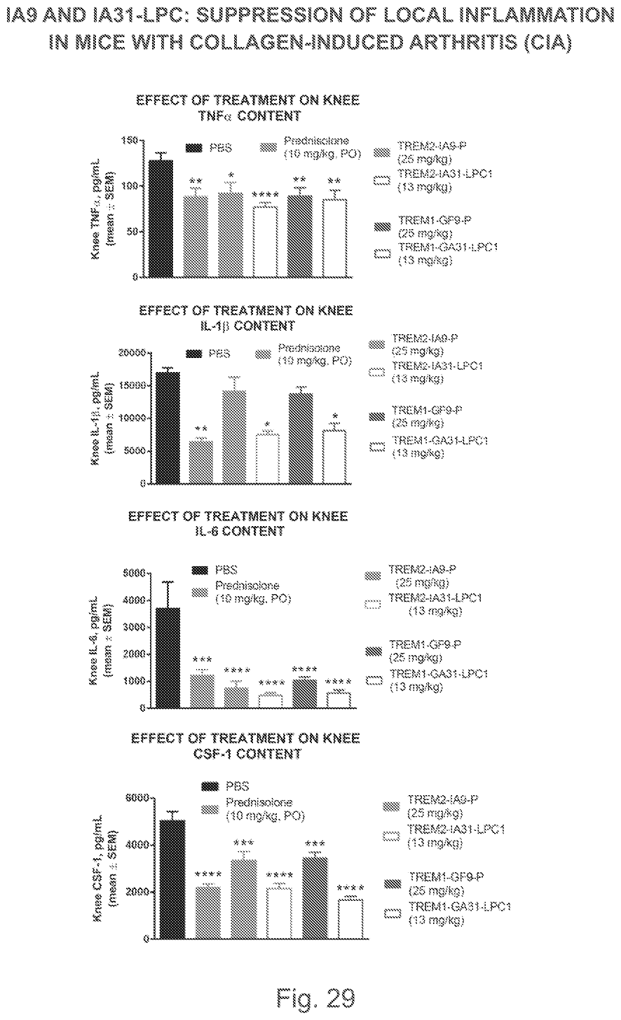

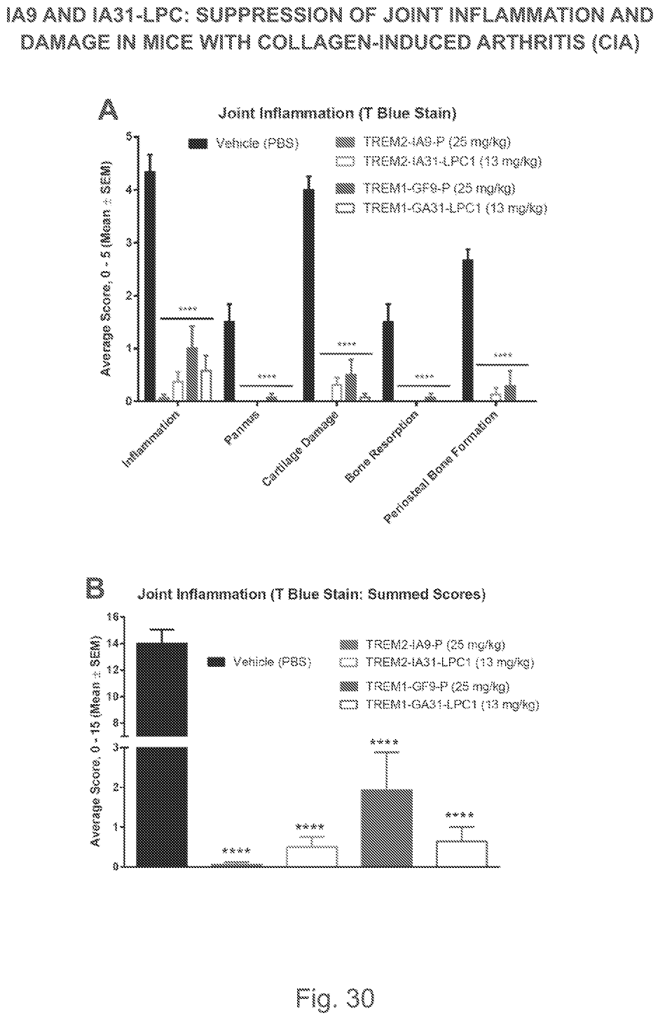

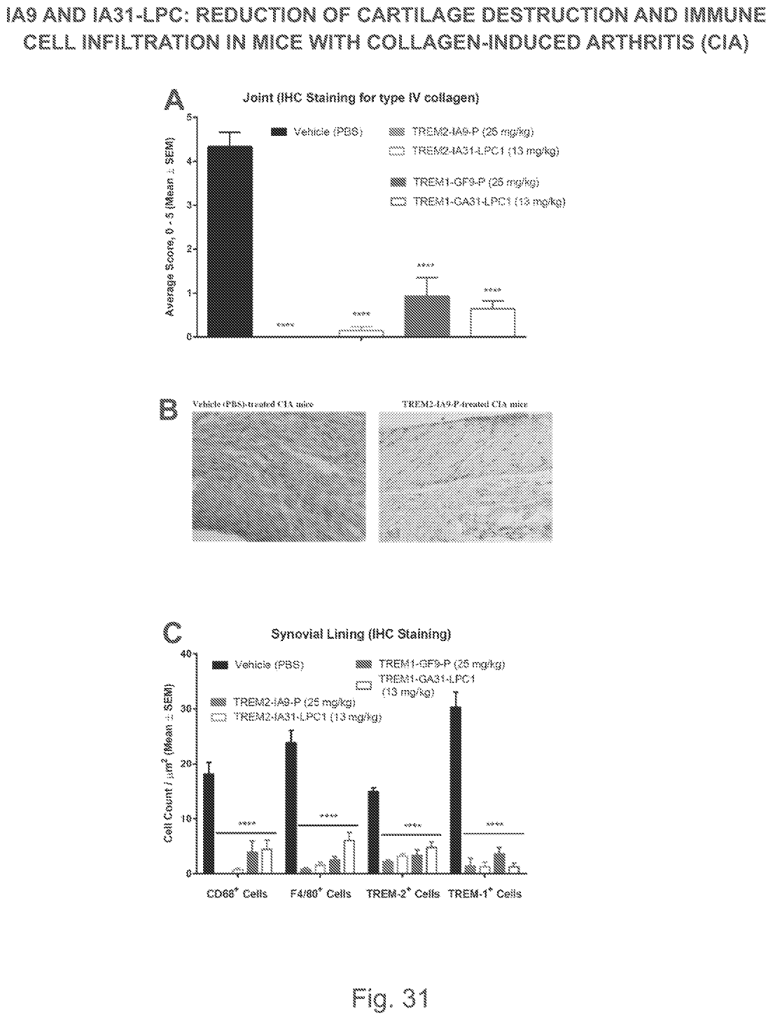

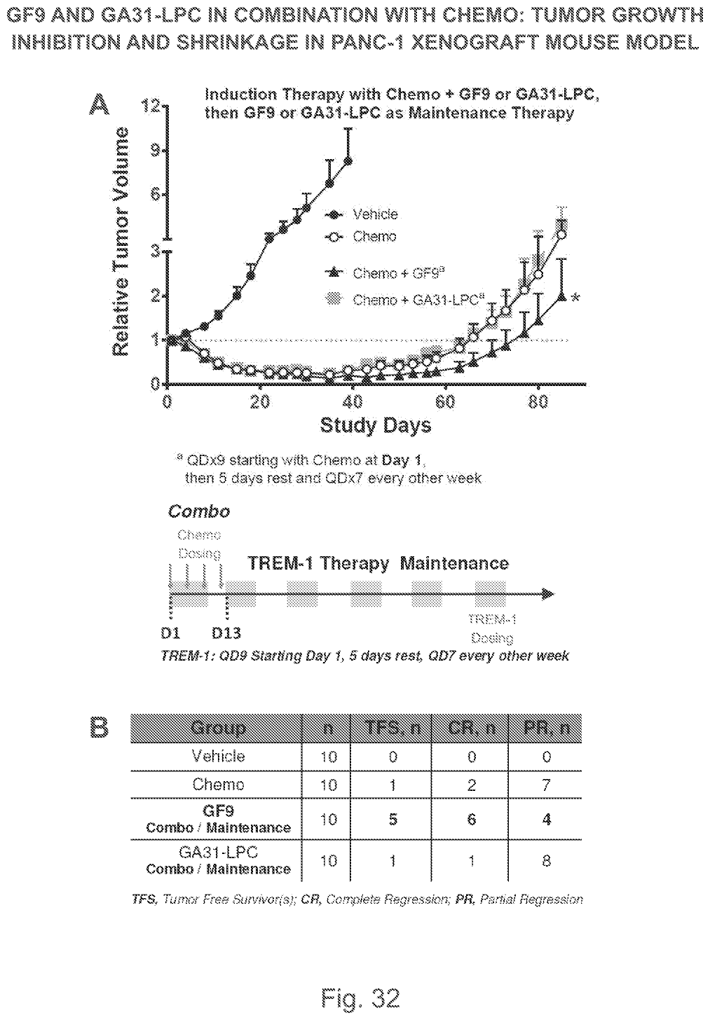

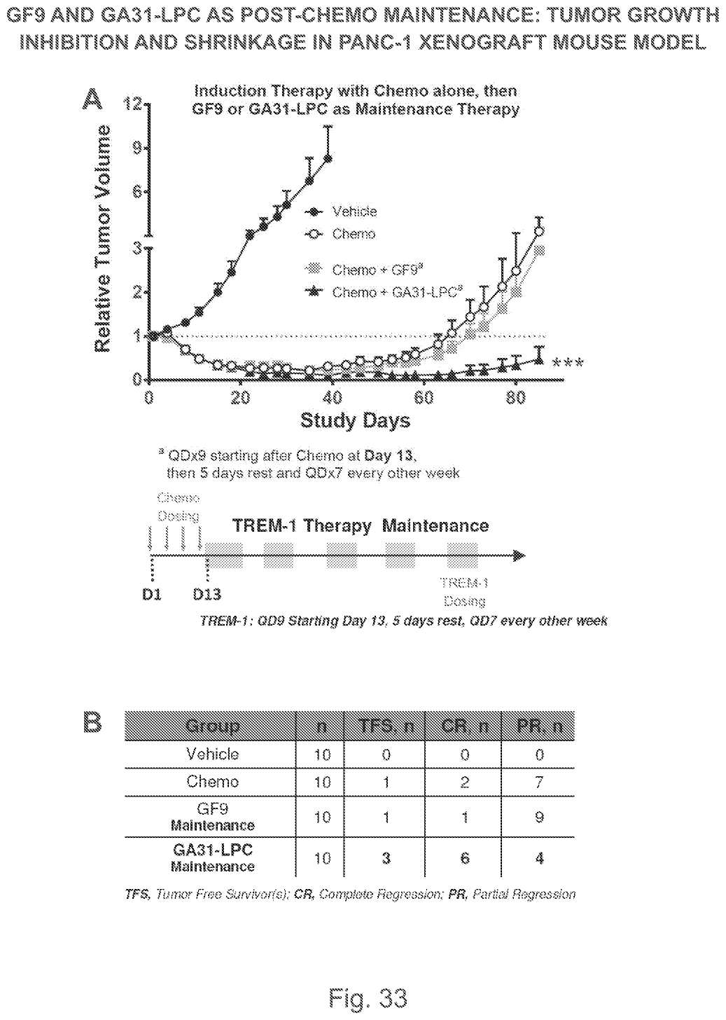

In certain embodiments, the present invention contemplates peptide variants, compositions and a method to prevent and/or treat bacteria-induced lung injuries including ARDS by suppressing lung inflammation by reducing the expression of at least one pulmonary cytokine by the peptide variants and compositions disclosed herein. In one embodiment, the peptide variants and compositions as described herein are the TREM-1 and/or TREM-2 inhibitor peptides including combinatorial TREM-1 and TREM-2 concurrent inhibitor peptides and compositions. In one embodiment, TREM-1 inhibitors comprise a free peptide GF9-P and a targeted LPC-based formulation of a trifunctional peptide GA31 (GA31-LPC3) as described herein. In one embodiment, a bacterial infection is a B. pertussis bacterial infection. In one embodiment, a TREM-1 receptor does not contribute to bacterial control in B. pertussis infection. See FIG. 17. In one embodiment, the at least one pulmonary cytokine reduced by using the peptide variants and compositions of the invention is CCL2 (or MCP-1). See FIG. 18. In one embodiment, the at least one pulmonary cytokine is CXCL3. See FIG. 18. In one embodiment, the at least one pulmonary cytokine is TNFa. See FIG. 18. In one embodiment, the treating comprises peptide variants and compositions as described herein that further reduces pulmonary inflammatory morphology symptoms and lung injury from the B. pertussis bacterial infection. In one embodiment, the treatment started Day 0 (prevention). In one embodiment, the treatment started Day 3 (treatment). See FIG. 19.

iii) Chemical-Induced Lung Injuries

Pulmonary toxicity is a cause of death in the individuals exposed to sulfur mustard (SM), and other chemicals affecting respiratory tract including, but not limited to, chlorine and/or phosgene. Treatment options for these chemicals are currently largely limited to supportive care.

As discussed herein, macrophages are key inflammatory cells that play role in the pathogenesis of many pulmonary ailments like asthma, pneumonia, chronic obstructive pulmonary disease, acute lung injury, and ARDS, which manifested as a result of long-term SM poisoning. Amplified, rapid acute inflammatory response in SM-affected lungs is also associated with cytokine storm (Malaviya et al. 2016, Weinberger et al. 2016, Sadeghi et al. 2020).

In SM toxicity, activated alveolar macrophages release pro-inflammatory mediators and chemokines (e.g. MCP, IL-8) that promote the accumulation of neutrophils. In an alveolar space, cytokines produced by alveolar macrophage (e.g. IL-1, IL-6, IL-8 and TNFa), stimulate chemotaxis and activate neutrophils. Neutrophils marginate through the interstitium into the alveolar space. Activated neutrophils can release pro-inflammatory molecules such as oxidants species, proteases, leukotrienes, and platelet-activating factor (PAF). TNFa production by activating neutrophils, further contributes to lung injury by releasing toxic mediators (Sadeghi et al. 2020).

In certain embodiments, the present invention contemplates peptide variants, compositions and a method to prevent or treat chemical-induced acute and chronic pulmonary injury and/or prevent mortality. In one embodiment, the chemical is SM. In one embodiment, the peptide variants and compositions as described herein are TREM-1 and/or TREM-2 inhibitor peptides including combinatorial TREM-1 and TREM-2 concurrent inhibitor peptides and compositions. In one embodiment, TREM-1 inhibitors comprise a free peptide GF9 (more specifically, GF9-P) and a targeted LPC-based formulation of a trifunctional peptide GA31 (GA31-LPC, more specifically, GA31-LPC3) as described herein. In certain embodiments, inhibitor peptide variants and compositions as described herein are tested in rats challenged with inhaled SM. In one embodiment, treatment of SM-challenged rats with GF9 and GA31-LPC is well-tolerable. See FIG. 20. In one embodiment, treatment with GF9 and GA31-LPC attenuates lung dysfunction and increases peripheral oxygen saturation. See FIG. 21. In one embodiment, treatment with GF9 and GA31-LPC significantly extends survival of SM-challenged rats. See FIG. 22. In some embodiments, this effect is more pronounced for GA31-LPC as compared with GF9. In certain embodiments, the median survival times are 5 days, 21 days and “undefined” (the latter means that more than 50% of the subjects are alive at the end of the study) for vehicle (more specifically, PBS, pH 7.4), GF9 and GA31-LPC, respectively. See FIG. 22. Although it is not necessary to understand the mechanism of an invention, it is believed that this difference can be explained by different effects of pan-TREM-1 inhibitors GF9 and a targeted TREM-2 inhibitor GA31-LPC on systemic and local inflammation in the pathogenesis of SM-induced lung injury.

### iv) Virus Infection-Induced Lung Injuries

Pandemic influenza A viruses and coronaviruses are relevant viruses that may cause lung injuries including ARDS (Frohlich 2021). Seven pandemic coronaviruses have been identified, four causing mild seasonal infections and three (MERS-CoV, SARS-CoV-1 and SARS-CoV-2) severe illness (Frohlich 2021).

Highly pathogenic SARS-CoV-1 and COVID-19-associated SARS-Cov-2 predominantly infect lower airways and causes fatal pneumonia (Channappanavar et al. 2017, Matthay et al. 2020). Severe pneumonia caused by pathogenic hCoVs is often associated with massive inflammatory cell infiltration (predominantly, macrophages) and elevated proinflammatory cytokines (cytokine storm) resulting in ARDS (Tisoncik et al. 2012, Channappanavar and Perlman 2017). IL-1b, TNFa, IL-6, MCP-1 and CSF-1 are elevated both in bronchoalveolar lavage (BAL) fluid and circulating plasma in COVID-19 patients (Guo et al. 2020).

Currently, most experimental COVID-19 therapeutics and vaccines are aimed at blocking the spread of the viral infection and treat early but not later (e.g., sepsis and ARDS) stages of the disease. No effective treatments of sepsis and ARDS are available, highlighting an urgent need for new therapies. Around 5% of COVID-19 patients develop severe ARDS that is a common cause of death related to COVID-19 in the ICU (Matthay et al. 2020, Poston et al. 2020, Thomas-Ruddel et al. 2020, Wujtewicz et al. 2020). Matthay et al. (2020); Poston et al. (2020); Thomas-Ruddel et al. (2020); and Wujtewicz et al. (2020).

In one embodiment, the present invention contemplates TREM-1 and/or TREM-2 peptide variants and related compositions and methods to provide an effective and well-tolerated virus infection therapy aimed to reduce the mortality rate and improve outcomes. In one embodiment, this therapy prevents and/or treats virus infection-induced lung injuries. In one embodiment, a virus infection is a COVID-19 infection. COVID-19 infections are believed to induce an amplified, rapid acute inflammatory response in the lungs associated with a cytokine storm. The peptide variants and compositions as described herein: i) reduce cytokine storms in vitro (FIG. 10); ii) reduce cytokine storms in vivo (FIG. 11); and iii) extend animal survival in two models of sepsis: mice with endotoxic shock induced by LPS (FIGS. 12, 13) and mice with polymicrobial sepsis induced with CS injection (FIGS. 12, 13). In certain embodiments, in combination with other data as described herein, these data suggest that the presently disclosed peptide variants, compositions and methods of treating can be used to suppress sepsis and septic shock and attenuate COVID-19-associated ARDS to reduce the mortality rate in COVID-19 patients and improve outcomes in infected risk groups.

### v) Pulmonary Fibrosis

Pulmonary fibrosis is the end stage of a broad range of heterogeneous interstitial lung diseases that occurs as a consequence of many types of severe lung injuries and is largely associated with inflammatory responses (Huang et al. 2021). Alveolar inflammation is important for amplifying host defenses in the lung, and alveolar macrophages contribute to this response (Reynolds 2005). Macrophages, neutrophils, eosinophils, and Th2 cells aggregate at the site of injury and release a large number of pro-inflammatory and pro-fibrotic cytokines/factors such as transforming growth factor-b (TGF-b), TNFa, matrix metalloproteinases (MMPs), tissue inhibitor of metalloproteinases (TIMPs), IL-1, IL-4, IL-5, IL-6, IL-13 and IL-17 (Reynolds 2005).

In certain embodiments, the present invention contemplates TREM-1 and/or TREM-2 peptide variants and related compositions and methods to prevent or treat pulmonary (lung) fibrosis as a consequence of various types of severe lung injuries including, but not limited to, those induced by radiation, chemicals, bacteria and/or viruses. In one embodiment, the method prevents mortality. In one embodiment, a common experimental model evaluates potential therapies to prevent and/or treat lung fibrosis and/or to prevent mortality. An exemplary common model is an inhaled bleomycin-induced lung fibrosis model. In one embodiment, the peptide variants and compositions of the invention were tested in a mouse model of inhaled bleomycin-induced lung fibrosis. In certain embodiments, peptide variants and compositions tested in the lung fibrosis model include TREM-1 and/or TREM-2 inhibitor peptides and compositions including combinatorial TREM-1 and TREM-2 concurrent inhibitor peptides and compositions. In one embodiment, the inhibitors comprise free peptides GF9, IA9 and GA18 (more specifically, GF9-D, IA9-P and GA18-D) and targeted LPC-based formulations of trifunctional peptides GA31 (GA31-LPC, more specifically, GA31-LPC3, GA31-LPC1 and GA31-LPC1-20) and IA31 (IA31-LPC, more specifically, IA31-LPC1) as described herein.

In one embodiment, preventative (starting Day 1), or therapeutic (starting Day 15), treatment with GF9-D, IA9-P, GA18-D, GA31-LPC3, GA31-LPC1, GA31-LPC1-20 or IA31-LPC1 significantly attenuates lung fibrosis induced in mice by inhaled bleomycin as evaluated by BALF total cell count and BALF total protein content. In one embodiment, BALF total cell count and BALF total protein content were analyzed at Day 7. In one embodiment, BALF total cell count and BALF total protein content were analyzed at Day 21. In one embodiment, BALF total cell count and BALF total protein content were analyzed at Day 28. See FIG. 23. In some embodiments, lung damage and fibrosis were evaluated by lung inflammation score. See FIG. 24. Two slides were made from each left lung tissue where one slide was stained using hematoxylin and eosin (H&E), while the other was stained with Masson' Trichrome. The severity of lung damage and fibrosis was evaluated and scored by a certified pathologist. In one embodiment, the severity of lung damage and fibrosis was analyzed at Day 7. In one embodiment, the severity of lung damage and fibrosis was analyzed at Day 21. In one embodiment, the severity of lung damage and fibrosis was analyzed at Day 28. See FIGS. 23, 24.

Fibrosis is a pathological scarring process that leads to destruction of organ architecture and impairment of organ function (Zeisberg et al. 2013). Chronic loss of organ function in most organs, including bone marrow, heart, intestine, kidney, liver, lung, and skin, is associated with fibrosis, contributing to an estimated one third of natural deaths worldwide. Effective therapies to prevent or to even reverse existing fibrotic lesions are not yet available in any organ. Although it is not necessary to understand the mechanism of an invention, it is believed that the peptide variants and compositions of this disclosure can be used to prevent or reverse tissue fibrosis in multiple organs. Example is the use of TREM-1 inhibitors to attenuate liver fibrosis caused by alcohol-induced liver injury as described in (Tornai et al. 2019, Sigalov 2020) and disclosed in WO/2020/036987.

### 2. Other Inflammation-Associated Diseases and Conditions

### a) Cancer

In certain embodiments, the present invention contemplates compositions and methods for treating cancer including but not limited to solid tumors and/or for post-treatment maintaining cancer patients to prevent and/or slow down cancer recurrence as disclosed in WO 2020/036987. In one embodiment, cancer is pancreatic cancer. In one embodiment, said compositions comprise cell surface receptor inhibitor peptide variants. In one embodiment, said cell surface receptor inhibitor peptide variants include, but are not limited to, TREM-1 and/or TREM-2 inhibitor peptide variants and combinatorial TREM-1 and TREM-2 concurrent inhibitor peptide variants.

In certain embodiments, the present invention contemplates compositions and methods for a combination therapy comprising cell surface receptor inhibitor peptide variants and a therapeutic drug for treating a cancer. In one embodiment, the cancer includes, but is not limited to, solid tumors and/or post-treatment residual cancer cells to prevent and/or slow down cancer recurrence as disclosed in WO 2020/036987. In one embodiment, said cell surface receptor inhibitor peptide variants are TREM-1 and/or TREM-2 inhibitor peptide variants and combinatorial TREM-1 and TREM-2 concurrent inhibitor peptide variants. Exemplary use of TREM-1 inhibitors, GF9 and GA31-LPC, in combination with standard chemotreatment is described in detail below, in the “Drug Combination Therapy” section. See FIGS. 32, 33.

### b) Arthritis and Other Inflammatory Diseases and Conditions

In certain embodiments, the present invention contemplates compositions and methods for treating autoimmune inflammatory diseases including, but not limited to, those disclosed in WO 2020/036987. In one embodiment, an inflammatory disease is rheumatoid arthritis (RA). In one embodiment, said compositions are cell surface receptor inhibitor peptide variants and compositions. In one embodiment, said cell surface receptor inhibitor peptide variants are TREM-1 and/or TREM-2 inhibitor peptide variants and combinatorial TREM-1 and TREM-2 concurrent inhibitor peptide variants. In one embodiment, said cell surface receptor inhibitor peptide variants are TCR, TREM-1 and/or TREM-2 inhibitor peptide variants and combinatorial TCR, TREM-1 and/or TREM-2 concurrent inhibitor peptide variants.

In certain embodiments, the present invention contemplates compositions and methods for a combination therapy comprising cell surface receptor inhibitor peptide variants and a therapeutic drug for treating an autoimmune inflammatory disease including, but not limited to, those disclosed in WO 2020/036987. In one embodiment, inflammatory disease is RA. In one embodiment, said compositions are cell surface receptor inhibitor peptide variants and compositions. In one embodiment, said cell surface receptor inhibitor peptide variants are TREM-1 and/or TREM-2 inhibitor peptide variants and combinatorial TREM-1 and TREM-2 concurrent inhibitor peptide variants. In one embodiment, said cell surface receptor inhibitor peptide variants are TCR, TREM-1 and/or TREM-2 inhibitor peptide variants and combinatorial TCR, TREM-1 and/or TREM-2 concurrent inhibitor peptide variants.

RA is a chronic inflammatory disorder characterized by chronic inflammation and T cell hyperactivation. In some people, the condition can damage a wide variety of body systems, including the joints, skin, eyes, lungs, heart and blood vessels. Current treatments include non-steroidal anti-inflammatory drugs (NSAIDs), corticosteroids, and disease modifying anti-rheumatic drugs (DMARDs) (Gaffo et al. 2006, Smolen et al. 2010). NSAIDs are toxic (Gaffo et al. 2006) and do not slow the clinical RA progression (O'Dell 2004). Corticosteroids are potent but toxic in high doses (O'Dell 2004). DMARDs include synthetic (e.g., methotrexate, MTX) and biologic (eg, TNFa and IL-1R blockers) agents (O'Dell 2004, Gaffo et al. 2006, Smolen et al. 2010). MTX is the most widely used non-biologic DMARD worldwide (Braun et al. 2009). Toxicities but not lack of efficacy are the most common cause of discontinuing MTX therapy (Suarez-Almazor et al. 2000, Wluka et al. 2000). Recently, biologics that block specific cytokines were approved to treat RA including TNF (e.g., Humira, Remicade) and IL-1 receptor (Kineret) blockers. Due to excessive immunosuppression, their use can cause fatal infections, malignancies and septic arthritis (Galloway et al. 2011, Atzeni et al. 2012, Choy et al. 2013). In RA patients, a higher dose of Remicade is necessary in patients with a high baseline TNF, whereas lower doses are sufficient for those with a low baseline TNF (Edrees et al. 2005, Takeuchi et al. 2011). This leads to a need for personalized treatment paradigms. Thus, there is an unmet need for efficient, safe and well tolerable RA therapy.

In patients with RA, monocytes immigrated into the RA synovial membrane differentiate to mature macrophages and the abundance and activation of these macrophages in the inflamed membrane correlates with the severity of RA (Tak et al. 1997, Kinne et al. 2000, Kinne et al. 2007). RA treatments predominantly target the sublining macrophages (Franz et al. 2005) while therapies that fail to reduce the number of synovial sublining macrophages are unlikely to be clinically effective (Franz and Burmester 2005, Bresnihan et al. 2007).

This makes it advantageous to deliver RA drugs directly to macrophages (Garrood et al. 2006, Kinne et al. 2007). Along with macrophages, T cells is another type of immune cells that play a role in RA (Cope et al. 2007). This makes it advantageous to use cell surface receptor inhibitor peptide variants and compositions of this disclosure that target either macrophages or T cells or both for treating RA. In one embodiment, said cell surface receptor inhibitor peptide variants and compositions are TREM-2 inhibitor peptide variants and compositions. In one embodiment, said cell surface receptor inhibitor peptide variants and compositions are combinatorial TREM-1 and TREM-2 inhibitor peptide variants and compositions. In one embodiment, said cell surface receptor inhibitor peptide variants and compositions are combinatorial TCR and TREM-1 inhibitor peptide variants. In one embodiment, said cell surface receptor inhibitor peptide variants and compositions are combinatorial TCR and TREM-2 inhibitor peptide variants. In one embodiment, said cell surface receptor inhibitor peptide variants and compositions are combinatorial TCR, TREM-1 and TREM-2 inhibitor peptide variants. See TABLES 2, 3 and 5.

The collagen-induced arthritis (CIA) mouse model is a commonly studied autoimmune model of rheumatoid arthritis (Brand et al. 2007). In one embodiment, the peptide variants and compositions of the present invention were tested in a CIA model. In one embodiment, peptide variants and compositions tested in the CIA model are the TREM-1 and/or TREM-2 inhibitor peptides and compositions including combinatorial TREM-1 and TREM-2 concurrent inhibitor peptides and compositions. In one embodiment, the inhibitors comprise free peptides GF9, IA9 and/or GA18 (more specifically, GF9-P, GF9-D, IA9-P and GA18-D) and targeted LPC-based formulations of trifunctional peptides GA31 (GA31-LPC, more specifically, GA31-LPC1) and IA31 (IA31-LPC, more specifically, IA31-LPC1) as described herein.

In one embodiment, therapeutic treatment starting on Day 28 post-CIA induction with GF9 (more specifically, GF9-P), IA9 (more specifically, IA9-P), GA31-LPC (more specifically, GA31-LPC1) and IA31-LPC (more specifically, IA31-LPC1) significantly suppresses arthritis in mice with CIA as evaluated by average clinical arthritic score (index). See FIG. 25. In some embodiments, the inhibitor peptides and compositions tested were free TREM-1 inhibitor peptide GF9 (more specifically, GF9-P and GF9-D), free TREM-2 inhibitor peptide IA9 (more specifically, IA9-P), combinatorial TREM-1 and TREM-2 inhibitor peptide GA18 (more specifically, GA18-D) and a 1:1 (by dose) mixture of GF9 and IA9 (more specifically, GF9-P and IA9-P). In one embodiment, these inhibitors and compositions significantly suppressed arthritis in mice with CIA as evaluated by average arthritic index (score). See FIG. 26. In one embodiment, GF9-P and GF9-D were equally effective in suppressing arthritis. In one embodiment, IA9-P and a 1:1 (by dose) mixture of GF9-P and IA9-P were more effective in reducing arthritis compared with GF9-P alone or GF9-D. In one embodiment, GA18-D was significantly more effective in reducing arthritis compared with 1:1 (by dose) mixture of GF9-P and IA9-P. Although it is not necessary to understand the mechanism of an invention, it is believed that targeting two TREM receptors, TREM-1 and TREM-2, may have a synergistic therapeutic effect as compared with targeting TREM-1 or TREM-2 alone. See FIG. 26.

In one embodiment, free peptides GF9 and IA9 (more specifically, GF9-P and IA9-P) and targeted LPC-based formulations of trifunctional peptides GA31 (GA31-LPC, more specifically, GA31-LPC1) and IA31 (IA31-LPC, more specifically, IA31-LPC1) described herein are all well-tolerated when administered for 14 days daily. See FIG. 27.

In one embodiment, daily treatment with free peptides GF9 and IA9 (more specifically, GF9-P and IA9-P) and targeted LPC-based formulations of trifunctional peptides GA31 (GA31-LPC, more specifically, GA31-LPC1) and IA31 (IA31-LPC, more specifically, IA31-LPC1) as described herein for 14 days starting at Study Day 28 significantly reduces systemic inflammation in mice with CIA as evaluated by analysis of proinflammatory cytokines IL-1b, IL-6 and CSF-1 in terminal plasma. See FIG. 28.

In one embodiment, daily treatment with free peptides GF9 and IA9 (more specifically, GF9-P and IA9-P) and targeted LPC-based formulations of trifunctional peptides GA31 (GA31-LPC, more specifically, GA31-LPC1) and IA31 (IA31-LPC, more specifically, IA31-LPC1) as described herein for 14 days starting at Study Day 28 significantly reduces local inflammation in mice with CIA as evaluated by analysis of proinflammatory cytokines TNFa, IL-1b, IL-6 and CSF-1 in knee joints. See FIG. 29.

In one embodiment, daily treatment with free peptides GF9 and IA9 (more specifically, GF9-P and IA9-P) and targeted LPC-based formulations of trifunctional peptides GA31 (GA31-LPC, more specifically, GA31-LPC1) and IA31 (IA31-LPC, more specifically, IA31-LPC1) as described herein for 14 days starting at Study Day 28 significantly suppresses joint inflammation and damage in mice with CIA as evaluated by scoring of inflammation, pannus, cartilage damage, bone resorption and periosteal bone formation in joints. See FIG. 30, Panel A. In one embodiment, daily treatment with free peptides GF9 and IA9 (more specifically, GF9-P and IA9-P) and targeted LPC-based formulations of trifunctional peptides GA31 (GA31-LPC, more specifically, GA31-LPC1) and IA31 (IA31-LPC, more specifically, IA31-LPC1) as described herein for 14 days starting at Study Day 28 significantly suppresses joint inflammation in mice with CIA as evaluated by scoring of T Blue-stained joint sections. See FIG. 30, Panel B.

In one embodiment, daily treatment with free peptides GF9 and IA9 (more specifically, GF9-P and IA9-P) and targeted LPC-based formulations of trifunctional peptides GA31 (GA31-LPC, more specifically, GA31-LPC1) and IA31 (IA31-LPC, more specifically, IA31-LPC1) as described herein for 14 days starting at Study Day 28 significantly reduces cartilage damage and immune cell infiltration. See FIG. 31. In one embodiment, cartilage destruction is analyzed and scored at Therapy Day 14 by using joint sections stained for type IV collagen (FIG. 31, Panel A). In one embodiment, exemplary images of the joint sections stained for type IV collagen are presented in FIG. 31, Panel B. In one embodiment, immune cell infiltration in synovial lining of joints is analyzed at Therapy Day 14 by immunohistochemical staining for immune cells (FIG. 31, Panel C). In one embodiment, immune cells are CD68-, F4/80-, TREM-2- and TREM-1-positive cells (FIG. 31, Panel C).

### 3. Diagnostic Imaging

In some embodiments, the cell surface inhibitor peptide variants and compositions disclosed herein including, but not limited to, those listed in TABLES 2, 3 and 5, are conjugated with an imaging probe to visualize the corresponding cell surface receptor(s) to evaluate its(their) expression in areas of interest. In one embodiment, the imaging probe is ⁶⁴Cu.

In one embodiment, imaging (visualization) of expressed TREM-1 and/or TREM-2 receptor levels using a positron emission tomography (PET) and/or other imaging techniques can be used to diagnose glioblastoma multiforme (GBM) and/or to select and monitor novel GBM therapies as described in (Johnson et al. 2017, Liu et al. 2019) and disclosed in WO 2017083682A1. In one embodiment, imaging (visualization) of expressed TREM-1 and/or TREM-2 receptor levels can be used to diagnose other TREM-1- and/or TREM-2 related types of cancer as well as to monitor therapies for these cancers.

Although it is not necessary to understand the mechanism of an invention, it is believed that targeted LPC-bound cell surface receptor inhibitor peptide variants and compositions of the present invention may cross BBB, BRB and BTB, thus delivering the inhibitors including, but not limited to those listed in TABLES 2, 3 and 5 to areas of interest in the brain, retina and tumor. While not being bound to any particular theory, it is believed that the brain-, retina-, and tumor-penetrating capabilities of these LPCs can be mediated by interaction of Scavenger receptor class B type I (SRBI) with the amino acid sequences that correspond to the sequences of human apo A-I helices 4 and/or 6 (Liu et al. 2002). See also, FIG. 8. As such, the cell surface receptor inhibitor peptide variants and compositions the present invention can be used to diagnose, treat and/or prevent those diseases and conditions, where delivery of these inhibitors to the brain, retina and/or tumor is needed (e.g., different types of cancer, Alzheimer's disease, stroke, neuroinflammation, retinopathy, etc.).

### 4. Drug Combination Therapy

In certain embodiments, the cell surface receptor inhibitor peptide variants and compositions of this disclosure may be administered in a combination therapy with other suitable treatment modalities. In certain embodiments, these modalities comprise those disclosed in U.S. Pat. Nos. 4,427,660; 9,161,988; 8,921,314; 9,173,891; 8,013,116; 9,273,111; 9,657,081; 9,815,883; 9,255,136; 11,186,636; 11,066,456: U.S. Pat. No. 11,124,567 (all of which are herein incorporated by reference) and WO 2020/036987.

Suitable treatment modalities for a cancer disease may include, without limitation, administration of radiation therapy, e.g., gamma radiation therapy. Other suitable treatment modalities for a cancer disease may include, for example, administering to a patient in combination with a vaccine, a chemotherapy agent, an immunotherapy agent, immunomodulatory agent, an additional therapeutic, or a combination thereof as disclosed in WO 2020/036987. In some embodiments, said cancer is pancreatic cancer. In one embodiment, a human pancreatic cancer PANC-1 xenograft mouse model is used to test anti-tumor activity of the inhibitor peptides and compositions of the invention. In some embodiments, said inhibitor peptides and compositions are TREM-1 inhibitor peptides and compositions. In one embodiment, said TREM-1 inhibitor peptides and compositions are free peptide GF9 and targeted LPC-bound trifunctional peptide GA31. In certain embodiments, water-insoluble peptides GF9 is solubilized in a mixture of propylene glycol, ethanol and Tween-80 (GF9-P). In one embodiment, GA31-LPC comprises a complex of GA31 with three lipid components (GA31-LPC3): 1-palmitoyl-2-oleoyl-sn-glycero-3-phosphocholine (POPC), cholesterol and cholesteryl oleate. See FIGS. 32-33.

In one embodiment, significant tumor growth inhibition and shrinkage and significantly increased number of tumor-free survivors (TFS) and complete regression (CR) were observed in human pancreatic tumor PANC-1 xenograft-carrying nude mice treated i.p. with 25 mg/kg free peptide GF9 (but not 13 mg/kg GA31-LPC) starting Day 1 in combination with chemo (80 mg/kg Gemcitabine i.p. Q3Dx4 and 30 mg/kg Abraxane intravenously, i.v., Q3Dx4) and then continuing as post-chemo maintenance therapy. In one embodiment, significant tumor growth inhibition and shrinkage and significantly increased number of tumor-free survivors (TFS) and complete regression (CR) were observed in human pancreatic tumor PANC-1 xenograft-carrying nude mice treated i.p. with 13 mg/kg GA31-LPC 25 mg/kg (but not free peptide GF9) starting Day 13 as post-chemo maintenance therapy (chemo: 80 mg/kg Gemcitabine i.p. Q3Dx4 and 30 mg/kg Abraxane intravenously, i.v., Q3Dx4). See FIGS. 32, 33.

While not being bound to any particular theory, it is believed that the observed dependence of anti-tumor activity on time of administration different for GF9 and GA31-LPC can be explained by different effects of pan-TREM-1 inhibitor GF9 and targeted TREM-1 inhibitor GA31-LPC on systemic and local inflammation in the pathogenesis of pancreatic cancer. See FIGS. 32, 33.

Through combination therapy, reduction of adverse drug reaction and potentiation of therapeutic activity are achievable by the combined effects of therapeutic agents having different mechanisms of action, including reduction of the non-sensitive cell population; prevention or delaying of occurrence of drug resistance; and dispersing of toxicity by means of a combination of drugs having different toxicities.

When a therapeutic agent used in combination with inhibitor peptide variants and compositions of the invention, it has a particular medication cycle. Specifically, the frequency of administration, dosage, time of infusion, medication cycle, and the like, may be determined properly according to individual cases, considering the kind of therapeutic agent, state of the patients, age, gender, etc.

In using the combination therapy of the present invention, the same dose as that usually given as a monotherapy or a slightly reduced dose (for example, 0.10-0.99 times the highest dose as a single agent) may be given through a normal administration route. The methods of the present invention will normally include medical follow-up to determine the therapeutic or prophylactic effect brought about in the patient undergoing treatment with the compound(s) and/or composition(s) described herein.

### EXPERIMENTAL

The following non-limiting examples are put forth so as to provide those of ordinary skill in the art with illustrative embodiments as to how the compositions, compositions, articles, devices, and/or methods claimed herein are made and evaluated. The examples are intended to be purely exemplary of the invention and are not intended to limit the scope of what the inventor regard as his invention. Efforts have been made to ensure accuracy with respect to numbers (e.g., amounts, temperature, etc.) but some errors and deviations should be accounted for. Standard methods of isolation, synthesis, modification, purification, and characterization of synthetic peptides and compositions are well-known in the art (see e.g., (Sigalov 2020); U.S. Pat. Nos. 4,749,742; 8,513,185; 9,981,004; 11,097,020; US 20210322508; US 20220047512; and US 2011/0256224 (all of which are herein incorporated by reference; and PCT/US2019/046392; and WO 2020/036987).

### Example 1

### Cells, Cell Lines and Reagents

Mouse macrophage cell line J774 and human pancreatic cancer cell line PANC-1 were purchased from the ATCC. Human PBMCs were purchased from Lonza. Sodium cholate, cholesteryl oleate and other chemicals were purchased from Sigma Aldrich Company. 1,2-dimyristoyl-sn-glycero-3-phosphocholine (DMPC), 1,2-dimyristoylsn-glycero-3-phospho-(1_-rac-glycerol) (DMPG), 1-palmitoyl-2-oleoyl-sn-glycero-3-phosphocholine (POPC), 1-palmitoyl-2-oleoyl-sn-glycero-3-phospho-(1′-rac-glycerol) (POPG), 1,2-dimyristoyl-snglycero-3-phosphoethanolamine-N-(lissamine rhodamine B sulfonyl) (Rho B-PE) and cholesterol were purchased from Avanti Polar Lipids.

### Example 2

### Peptide Synthesis and Formulations

The following synthetic peptides were made by AmbioPharm, Inc.:
a) 9-mer peptide
   GFLSKSLVF (human TREM-1 213-221, GF9)
b) 9-mer peptide
   IFLIKILAA (human TREM-2 182-190, IA9)
c) 18-mer peptide
   GFLSKSLVFIFLIKILAA (GA18)
d) 31-mer unmodified peptides
   GFLSKSLVFPLGEEMRDRARAHVDALRTHLA (GA3lun), and
   IFLIKILAAPLGEEMRDRARAHVDALRTHLA (IA31un)
e) 31-mer methionine sulfoxidized peptides
   GFLSKSLVFPLGEEM(O)RDRARAHVDALRTHLA (GA31), and
   IFLIKILAAPLGEEM(O)RDRARAHVDALRTHLA (IA31)

In one embodiment, water-insoluble GF9 and IA9 were dissolved in an aqueous solution of propylene glycol, ethanol and Tween-80 (GF9-P and IA9-P, respectively). In one embodiment, water-insoluble IA9 is not soluble an aqueous solution of Dexolve. In one embodiment, water-insoluble GF9 and GA18 were dissolved in an aqueous solution of Dexolve (GF9-D and GA18-D, respectively). In one embodiment, sterile filtered GF9 (more specifically, GF9-P and GF9-D), IA9 (more specifically, IA9-P) and GA18 (more specifically, GA18-D-D) solutions were stable at 4° C. for at least, up to 6 months, as analyzed by reversed-phase high-performance liquid chromatography (RP-HPLC).

### Example 3

Lipopeptide Complexes HDL-mimicking lipopeptide complexes (LPC) of spherical morphology loaded with GA31 or IA31 (GA31-LPC and IA31-LPC, respectively) with unmodified or sulfoxidized methionine residues were synthesized using the sodium cholate dialysis procedure, purified and characterized essentially as previously described in (Sigalov 2014b, Shen and Sigalov 2016, Shen and Sigalov 2017b, Shen and Sigalov 2017a, Rojas et al. 2018, Tornai et al. 2019, Gallop et al. 2021) Sigalov (2014); Shen et al. (2016); and Shen and Sigalov (2017); and disclosed in U.S. Pat. No. 11,097,020; US 2021/0322508; US 2022/0047512; and US 2011/0256224 (all of which are herein incorporated by reference); and WO 2020/036987.

In one embodiment, the molar ratio was 56:2.5:0.6:1:96 for POPC:cholesterol:cholesteryl oleate:GA31(or IA31):sodium cholate (GA31-LPC3 or IA31-LPC3, respectively). In one embodiment, the molar ratio was 56:1:96 for POPC:GA31(or IA31):sodium cholate (GA31-LPC1 or IA31-LPC1, respectively). In one embodiment, the molar ratio was 20:1:96 for POPC:GA31:sodium cholate (GA31-LPC1-20).

In one embodiment, to synthesize GA31-LPC3, POPC, cholesterol, and cholesteryl oleate in organic solvents were mixed, dried in a stream of argon, and placed under vacuum for 8 h. In one embodiment, to synthesize GA31-LPC1, POPC in chloroform was dried in a stream of argon, and placed under vacuum for 8 h. To synthesize fluorescently labeled LPC, rhodamine B-PE in chloroform was also added to a lipid mixture. To synthesize Gd-labeled LPC, 14:0 PE-DTPA (Gd) in chloroform was also added to a lipid mixture. Then, lipid films were dispersed in Tris-buffered saline-EDTA (TBS-EDTA, pH 7.4), sonicated for 5 min and incubated for 30 min at 30° C. To the dispersed lipids, aqueous solution of either methionine sulfoxidized or unmodified GA31 was added. Amount of GA31 was controllably varied in different preparations. Then, sodium cholate solution was added and the mixture was incubated at 30° C. for 3 h, followed by extensive dialysis against PBS to remove sodium cholate. The same procedures was used to prepare LPC with either methionine sulfoxidized or unmodified IA31 (IA31-LPC3 and IA31-LPC1). In one embodiment, hydrodynamic radius of GA31-LPC3 was 70 nm as measured by dynamic light scattering (DLS). In one embodiment, hydrodynamic radius of GA31-LPC1-20 was 50 nm as measured by DLS. In one embodiment, hydrodynamic radius of IA31-LPC3 was 65 nm as measured by DLS. In one embodiment, hydrodynamic radius of IA31-LPC1 was 45 nm as measured by DLS. In one embodiment, as analyzed by DLS, size exclusion chromatography (SEC), electron microscopy and RP-HPLC, sterile filtered GA31-LPC and IA31-LPC formulations were stable at 4° C. for at least, up to 6 months.

### Example 4

### In Vitro Macrophage Endocytosis

In vitro studies of macrophage endocytosis of fluorescently labeled GA31-LPC (more specifically, GA31-LPC3, GA31-LPC1, and GA31-LPC1-20) and IA31-LPC (more specifically, IA31-LPC1) were performed using the standard methods well known in the art. See, e.g. (Sigalov 2014b, Shen and Sigalov 2016, Shen and Sigalov 2017b, Shen and Sigalov 2017a, Rojas et al. 2018, Tornai et al. 2019, Gallop et al. 2021). In one embodiment, GA31 and IA31 in GA31-LPC and IA31-LPC were labeled with DyLight 405. More experimental details are disclosed in the FIG. 9 legend.

This example demonstrates that sulfoxidation of methionine residues in GA31 and IA31 targets the corresponding GA31-LPC and IA31-LPC to macrophages and enhances their macrophage uptake (endocytosis). See FIG. 9.

### Example 5

### In Vitro Cytokine Release

In vitro studies of cytokine release by LPS-stimulated human PBMCs in the presence of GF9, IA9, GA18, GA31-LPC and IA31-LPC were performed using the standard methods well known in the art. See, e.g. (Ngkelo et al. 2012). More experimental details are disclosed in the FIG. 10 legend.

This example demonstrates that GF9, IA9, GA18, GA31-LPC and IA31-LPC all significantly suppress inflammatory response by LPS-stimulated human PBMC. In one embodiment, it further demonstrates that GF9-D and GA18-D are at least as effective as GF9-P and IA9-P suggesting that therapeutically effective clinically relevant formulations of these peptides can be prepared. In one embodiment, it further demonstrates that GA31-LPC1 and GA31-LPC1-20 are at least as effective as GA31-LPC3 suggesting that the number of lipid components in GA31-LPC can be reduced without losing therapeutic efficacy of this complex. In one embodiment, it further demonstrates that TREM-2 inhibitor peptide IA9, LPC-bound trifunctional peptide IA31 (more specifically, IA31-LPC1) and combinatorial TREM-1 and TREM-2 concurrent inhibitor peptide GA18 of the invention are all effective in suppression of LPS-induced cytokine release in vitro. See FIG. 10.

In one embodiment, other cell surface receptor inhibitor peptide variants and compositions of this disclosure can be tested in this cell model for their ability to suppress inflammatory response.

### Example 6

### Efficacy Testing in LPS-Challenged Mice

Studies of GF9, IA9, GA18, GA31-LPC and IA31-LPC in mice with LPS-induced endotoxic shock were performed using the standard methods well known in the art. See, e.g. (Sigalov 2014b). More experimental details are disclosed in the FIGS. 11-13 legends.

This example demonstrates that GF9, IA9, GA18, GA31-LPC and IA31-LPC all significantly suppress systemic inflammation and extend animal survival when administered pre- or post-LPS challenge. In one embodiment, it further demonstrates that LPC-bound GA31 and IA31 are more effective than free peptides GF9, IA9 and GA18. In one embodiment, it further demonstrates that GA31-LPC and IA31-LPC significantly protect mice against LPS-induced death even when administered 3 hrs after LPS challenge. In one embodiment, it further demonstrates that GF9-D and GA18-D are at least as effective as GF9-P and IA9-P suggesting that therapeutically effective clinically relevant formulations of these peptides can be prepared. In one embodiment, it further demonstrates that GA31-LPC1 and GA31-LPC1-20 are at least as effective as GA31-LPC3 suggesting that the number of lipid components in GA31-LPC can be reduced without losing therapeutic efficacy of this complex. In one embodiment, it further demonstrates that TREM-2 inhibitor peptide IA9, LPC-bound trifunctional peptide IA31 (more specifically, IA31-LPC1) and combinatorial TREM-1 and TREM-2 concurrent inhibitor peptide GA18 of the invention are all effective in suppression of inflammation and survival extension in mice with LPS-induced endotoxic shock. See FIGS. 11-13.

In one embodiment, other cell surface receptor inhibitor peptide variants and compositions of this disclosure can be tested in this animal model for their ability to suppress inflammatory response and extend animal survival.

### Example 7

### Efficacy Testing in Cecal Slurry (CS)-Challenged Mice

Studies of GF9, IA9, GA18, GA31-LPC and IA31-LPC in mice with CS-induced polymicrobial sepsis were performed using the standard methods well known in the art. See, e.g. (Starr et al. 2014). More experimental details are disclosed in the FIGS. 14-15 legends.

This example demonstrates that GF9, IA9, GA18, GA31-LPC and IA31-LPC all significantly extend animal survival when administered pre- or post-CS challenge. In one embodiment, it further demonstrates that LPC-bound GA31 and IA31 are more effective than free peptides GF9, IA9 and GA18. In one embodiment, it further demonstrates that GA31-LPC and IA31-LPC significantly protect mice against CS-induced death even when administered 12 hrs post-CS challenge. In one embodiment, it further demonstrates that GF9-D and GA18-D are at least as effective as GF9-P and IA9-P suggesting that therapeutically effective clinically relevant formulations of these peptides can be prepared. In one embodiment, it further demonstrates that GA31-LPC1 and GA31-LPC1-20 are at least as effective as GA31-LPC3 suggesting that the number of lipid components in GA31-LPC can be reduced without losing therapeutic efficacy of this complex. In one embodiment, it further demonstrates that TREM-2 inhibitor peptide IA9, LPC-bound trifunctional peptide IA31 (more specifically, IA31-LPC1) and combinatorial TREM-1 and TREM-2 concurrent inhibitor peptide GA18 of the invention are all effective in survival extension in mice with CS-induced polymicrobial sepsis. See FIGS. 14-15.

In one embodiment, other cell surface receptor inhibitor peptide variants and compositions of this disclosure can be tested in this animal model for their ability to suppress inflammatory response and extend animal survival.

### Example 8

### Efficacy Testing in B. Pertussis-Infected Mice

Studies of GF9 and GA31-LPC in mice challenged intranasally with B. pertussis were performed using the standard methods well known in the art. See, e.g. (Gallop et al. 2021). More experimental details are disclosed in the FIGS. 17-19 legends.

This example demonstrates that GF9 and GA31-LPC (more specifically, GF9-P and GA31-LPC3) do not affect bacterial control in B. pertussis virus infection. In one embodiment, it further demonstrates that GF9 and GA31-LPC significantly suppress lung inflammation in B. pertussis-infected mice as evaluated by tissue proinflammatory cytokine levels and lung pathology score. In one embodiment, it further demonstrates that GA31-LPC at 13 mg/kg dose is at least as effective as GF9 at 25 mg/kg dose. See FIGS. 17-19.

In one embodiment, other cell surface receptor inhibitor peptide variants and compositions of this disclosure can be tested in this animal model for their effect on bacterial control. In one embodiment, said cell surface receptor inhibitor peptide variants are TREM-2 inhibitor peptide variants and combinatorial TREM-1 and TREM-2 concurrent inhibitor peptide variants. In one embodiment, said TREM-2 inhibitor peptide variants and combinatorial TREM-1 and TREM-2 concurrent inhibitor peptide variants are anticipated do not affect bacterial control.

### Example 9

### Tolerability and Efficacy Testing in Sulfur Mustard (SM)-Challenged Mice

Studies of GF9 and GA31-LPC in rats challenged with inhaled SM were performed using the standard methods well known in the art. See, e.g. (Perry et al. 2021). More experimental details are disclosed in the FIGS. 20-22 legends.

This example demonstrates that GF9 and GA31-LPC (more specifically, GF9-P and GA31-LPC3) both are well-tolerated in rats challenged with inhaled SM. In one embodiment, it further demonstrates that GA31-LPC and GF9 attenuate SM challenge-induced lung dysfunction and oxygen saturation. In one embodiment, it further demonstrates while GF9 and GA31-LPC both extend survival of SM-challenged rats, GA31-LPC is more effective and exhibits longer and more effective protection compared with that of GF9. In one embodiment, it further demonstrates that GA31-LPC significantly extends survival for at least two weeks after the dosing is completed. See FIGS. 20-22.

In one embodiment, other cell surface receptor inhibitor peptide variants and compositions of this disclosure can be tested in this animal model for their tolerability and efficacy in prevention and treatment of SM-induced lung injury. In one embodiment, said cell surface receptor inhibitor peptide variants are TREM-2 inhibitor peptide variants and combinatorial TREM-1 and TREM-2 concurrent inhibitor peptide variants. In one embodiment, said TREM-2 inhibitor peptide variants and combinatorial TREM-1 and TREM-2 concurrent inhibitor peptide variants are anticipated be well tolerable. In one embodiment, said TREM-2 inhibitor peptide variants and combinatorial TREM-1 and TREM-2 concurrent inhibitor peptide variants are anticipated to attenuate SM challenge-induced lung dysfunction and oxygen saturation and extend survival of SM-challenged animals.

### Example 10

### Efficacy Testing in Intratracheal Bleomycin-Challenged Mice

Studies of GF9 (more specifically, GF9-D), IA9 (more specifically, IA9-P), GA18 (more specifically, GA18-D), GA31-LPC (more specifically, GA31-LPC3, GA31-LPC1 and GA31-LPC1-20) and IA31-LPC (more specifically, IA31-LPC1) in mice with pulmonary fibrosis induced by intratracheal bleomycin were performed using the standard methods well known in the art. See, e.g. (Lawson et al. 2005, Liu et al. 2017). More experimental details are disclosed in the FIGS. 23-24 legends.

This example demonstrates that GF9, IA9, GA18, GA31-LPC and IA31-LPC are all effective in prevention and treatment of intratracheal bleomycin-induced pulmonary fibrosis as evaluated by BALF total cell count, BALF total protein content and lung pathology. In one embodiment, it further demonstrates that GF9-D and GA18-D are at least as effective as GF9-P and IA9-P suggesting that therapeutically effective clinically relevant formulations of these peptides can be prepared. In one embodiment, it further demonstrates that GA31-LPC1 and GA31-LPC1-20 are at least as effective as GA31-LPC3 suggesting that the number of lipid components in GA31-LPC can be reduced without losing therapeutic efficacy of this complex. In one embodiment, it further demonstrates that TREM-2 inhibitor peptide IA9, LPC-bound trifunctional peptide IA31 (more specifically, IA31-LPC1) and combinatorial TREM-1 and TREM-2 concurrent inhibitor peptide GA18 of the invention are all effective in prevention and treatment of intratracheal bleomycin-induced pulmonary fibrosis. See FIGS. 23-24.

In one embodiment, other cell surface receptor inhibitor peptide variants and compositions of this disclosure can be tested in this animal model for their ability to prevent and treat intratracheal bleomycin-induced pulmonary fibrosis.

### Example 11

Efficacy Testing in Mice with Collagen-Induced Arthritis (CIA)

Studies of GF9 (more specifically, GF9-P and GF9-D), IA9 (more specifically, IA9-P), GA18 (more specifically, GA18-D), GA31-LPC (more specifically, GA31-LPC1) and IA31-LPC (more specifically, IA31-LPC1) in mice with CIA were performed using the standard methods well known in the art. See, e.g. (Shen and Sigalov 2017b). More experimental details are disclosed in the FIGS. 25-31 legends.

This example demonstrates that GF9, IA9, GA31-LPC and IA31-LPC are all well-tolerable and effective in suppression of systemic and local inflammation and in protection against joint damage induced by collagen as evaluated by scoring macroscopic signs of arthritis, analysis of systemic and local proinflammatory cytokine release and histopathologic and immunohistochemical evaluation of joints. In one embodiment, it further demonstrates that GF9-D is at least as effective as GF9-P suggesting that therapeutically effective clinically relevant formulations of this peptide can be prepared. In one embodiment, it further demonstrates that GA18-D is significantly more effective compared with concurrent treatment with GF9-P and IA9-P (1:1 by dose). In one embodiment, it further demonstrates that TREM-2 inhibitor peptide IA9 (more specifically, IA9-P), LPC-bound trifunctional peptide IA31 (more specifically, a clinically relevant formulations IA31-LPC1) and combinatorial TREM-1 and TREM-2 concurrent inhibitor peptide GA18 (more specifically, a clinically relevant formulation GA18-D) of the invention are all effective in prevention and treatment of collagen-induced inflammatory response and joint damage. See FIGS. 25-31.

In certain embodiments, other cell surface receptor inhibitor peptide variants and compositions of this disclosure can be tested in this animal model for their ability to prevent and treat arthritis. In one embodiment, said cell surface receptor inhibitor peptide variants and compositions are combinatorial TCR and TREM-1 concurrent inhibitor peptide variants and compositions. In one embodiment, said cell surface receptor inhibitor peptide variants and compositions are combinatorial TCR and TREM-2 concurrent inhibitor peptide variants and compositions. In one embodiment, said cell surface receptor inhibitor peptide variants and compositions are combinatorial TCR, TREM-1 and TREM-2 concurrent inhibitor peptide variants and compositions. In one embodiment, these inhibitor peptide variants and compositions are all anticipated to prevent and treat arthritis and protect joints from damage in animals with CIA.

### Example 12

Efficacy Testing in Mice with PANC-1 Xenografts

Studies of GF9 (more specifically, GF9-P) and GA31-LPC (more specifically, GA31-LPC3) in nude mice with subcutaneously inoculated human pancreatic cancer PANC-1 cells were performed using the standard methods well known in the art. See, e.g. (Shen and Sigalov 2017a). More experimental details are disclosed in the FIGS. 32-33 legends.

This example demonstrates significant antitumor activity of GF9 (but not GA31-LPC) when administered starting Day 1 in combination with a standard chemotreatment and then continuing as maintenance therapy. It further demonstrates significant antitumor activity of GA31-LPC (but not GF9) when administered starting Day 13 as post-chemo maintenance therapy. See FIGS. 32-33.

In certain embodiments, other formulations of GF9 (more specifically, GF9-D) and GA31-LPC (more specifically, GA31-LPC1) can be tested in this model for their anti-tumor efficacy. In one embodiment, it is anticipated that GF9-D and GA31-LPC1 are as effective as GF9-P and GA31-LPC3 in tumor growth inhibition and tumor shrinkage and in increasing number of TFS and CR.

In certain embodiments, other cell surface receptor inhibitor peptide variants and compositions of this disclosure can be tested in this animal model for their ability to inhibit tumor growth, promote tumor shrinkage and increase number of TFS and CR. In one embodiment, said cell surface receptor inhibitor peptide variants and compositions are TREM-2 inhibitor peptide variants and compositions. In one embodiment, said cell surface receptor inhibitor peptide variants and compositions are combinatorial TREM-1 and TREM-2 concurrent inhibitor peptide variants and compositions. In one embodiment, said TREM-2 inhibitor peptide variants and compositions and combinatorial TREM-1 and TREM-2 concurrent inhibitor peptide variants and compositions are all anticipated to inhibit tumor growth, promote tumor shrinkage and increase number of TFS and CR in cancer animals.

### Example 13

### Acute Lung Injury Efficacy Testing

Studies of the peptide variants and compositions of this disclosure in mice with LPS-induced lung injury and neutrophilic inflammation are performed using the standard methods well known in the art. See, e.g. (Yuan et al. 2016, Sadikot et al. 2017).

Briefly, male wild-type C57BL/6J mice (6-8 wk, weighing 20-30 g) are randomized into groups. In all “LPS” groups, 10 mg/kg LPS (E. coli 026:B6; Sigma) is administered at T=0 by oropharyngeal aspiration. Administered via a DeVilbiss disposable nebulizer (at the continuous air flow rate of 10 ft3/h) over 1 h to induce ALI. Control animals receive vehicle (PBS), respectively.

Mice are i.p. or subcutaneously treated with the peptide variants and compositions of the invention at various doses and timepoints before (preventative model) or after LPS challenge (therapeutic model). In certain embodiments, said peptide variants and compositions of the invention are GF9, IA9, GA18, GA31-LPC or IA31-LPC.

Terminal procedures are performed at 6 or 12 h post-LPS. Pre-terminal plasma is collected for cytokine analysis. Lung lavage fluid is centrifuged at 400 g for 10 min. The supernatant is kept at 70° C., the cell pellet is suspended in serum-free RPMI 1640, and total cell counts are determined on a grid hemocytometer. BAL procedures (4 mL×3 occasions) are performed. BALF total and differential cell counts are analyzed. Myeloperoxidase (MPO) activity is analyzed in lung samples. TREM-1, IL-1b, TNFa, IL-6, CSF-1, IL-10 and keratinocytes-derived chemokine (KC) involved in ARDS are analyzed in BAL supernatant samples and plasma as described (Yuan et al. 2016, Gong et al. 2020). Lung wet-to-dry ratios are determined. Lavaged lungs are inflation fixed in 10% 5 neutral buffered formalin. Sections (3/left lung/animal; 6/right lung/animal) are taken from each lung lobe and stained by Hematoxylin and Eosin (H&E) for assessment of inflammation.

In certain embodiments, cell surface receptor inhibitor peptide variants and compositions of this disclosure tested in this animal model are anticipated to ablate neutrophilic inflammation and attenuate lung injury. In one embodiment, said cell surface receptor inhibitor peptide variants and compositions are TREM-2 inhibitor peptide variants and compositions. In one embodiment, said cell surface receptor inhibitor peptide variants and compositions are combinatorial TREM-1 and TREM-2 concurrent inhibitor peptide variants and compositions.

### Example 14

Synthesis of Imaging probe ([64Cu])-Conjugated Peptides in Free and LPC-Bound Form

This example demonstrates synthesis of a TREM-2-related trifunctional peptide IA31 compound comprising an imaging probe [⁶⁴Cu] ([64Cu]IA31).

The first step is to synthesize the trifunctional compound comprising two domains where one domain is a TREM-2 inhibitory peptide sequence IA9, whereas another domain is either a [64Cu]-labeled 22 amino acids-long apolipoprotein A-I helix 6 peptide sequence PA22 with sulfoxidized methionine residue ([⁶⁴Cu]IA31) or a [⁶⁴Cu]-labeled 22 amino acids-long apolipoprotein A-I helix 4 peptide sequence PE22 with sulfoxidized methionine residue ([64Cu]IE31).

Although it is not necessary to understand the mechanism of an invention, it is believed that a 22 amino acids-long apolipoprotein A-I helix 6 and 4 peptide sequence domains with sulfoxidized methionine residues will assist in the self-assembly of LPC upon binding to lipid or lipid mixtures and target the [64Cu]IA31-LPC (or [64Cu]IE31-LPC) particles to macrophages, whereas peptide sequence domain IA9 will assist in the self-insertion of [64Cu]IA31 (or [64Cu]IE31) released from the corresponding the released from [64Cu]IA31-LPC (or [64Cu]IE31-LPC) particles upon endocytosis by cells of interest (e.g., tissue macrophages: tumor-associated macrophages—TAMs; Kupffer cells, etc.) into the cell membrane and subsequent colocalization of [64Cu]IA31 (or [64Cu]IE31) with TREM-2 expressed on these cells. This is believed to result in TREM-2 inhibition along with [[64Cu]IA31- or [64Cu]IE31-PET signal in the macrophage-rich areas of interest allowing for visualization of macrophage-mediated inflammation (e.g., neuroinflammation, inflamed atherosclerotic plaques, intratumoral inflammation, etc.).

The trifunctional peptide compositions of this disclosure containing conjugated [64Cu]can be synthesized using an alternative method disclosed in WO 2017083682A1. DOTA (1,4,7, 10-tetraazacyclododecane-1,4,7,10-tetraacetic acid) conjugation is performed according to established protocols, using metal-free buffers. After conjugation, matrix-assisted laser desorption/ionization (MALDI) mass spectrometry is conducted to determine the average number of DOTA molecules conjugated per the trifunctional peptide variant and composition (e.g., IA31). Subsequently, the DOTA-conjugated IA31 (or IE31) is radiolabeled with [64Cu] by incubating it in a [64Cu]CuCI2 solution (pH 5.5) at 37° C. for one hour with continual shaking. The reaction is purified via a NAP5 column and specific activity of the final labeled IA31 (or IE31) is determined via size exclusion HPLC. [64Cu]IA31 (or [64Cu]IE31) can be synthesized with high specific radioactivity (>75 GBq/μ), radiochemical purity (>99%), and labeling efficiency (50-75%), which is sufficient for in vitro and in vivo use.

LPC-bound [64Cu]-labeled trifunctional peptide variant and composition are prepared, purified and characterized using the methods and procedures described herein and disclosed in U.S. Pat. No. 11,097,020; US 2021/0322508; US 2022/0047512; and US 2011/0256224 (all of which are herein incorporated by reference); and WO 2020/036987.

### Example 15

### Use of [64Cu]1A31 in Imaging of Neuroinflammation

This example demonstrates the feasibility of using [64Cu]IA31 to visualize neuroinflammation in vivo, PET/CT imaging of middle cerebral artery occlusion (MCAo) mice as disclosed in WO 2017083682A1.

The MCAo model of cerebral ischemia is selected since the time-course of macrophage infiltration and microglial activation in the brain infarct is well documented, and because this model is commonly used to evaluate candidate microglial/macrophage-PET tracers. B6 mice (n=3), MCAo (n=9), and sham (n=9) mice are injected via tail vein with 80-85 1 of [64Cu]IA31-LPC in a saline solution (0.9% sodium chloride) and imaged using PET/CT at 3h post-injection. They are imaged again at 19h post-injection, which is 1.5-2 days after surgery/stroke.

In one embodiment, [64Cu]IE31 can be tested in this model for imaging of inflammation.

### Example 16

### Pneumonia Efficacy Testing

Studies of the peptide variants and compositions of this disclosure to demonstrate that said peptide variants and compositions (therapeutic agents, TAs) are effective in attenuating lung and systemic inflammatory responses of the pneumonia are performed using the standard methods. See, e.g. (Gibot et al. 2006a, Hommes et al. 2014).

Briefly, a nonmucoid Pseudomonas aeruginosa strain (serotype PAO1, ATCC BAA-47) is used for all studies. On the day of the experiment, and a 0.5 mL/kg of the P aeruginosa solution in saline at a concentration of 7×10⁸-8×10⁸ cfu/mL is used for intratracheal inoculation in adult male Wistar rats (350-380 g). Samples of bronchoalveolar lavage (BAL) fluid and lung homogenate are obtained aseptically for culture in rats with pneumonia 24 h after the bacterial instillation. Rats are anesthetized for a brief period. A median incision is made in the anterior neck to expose the trachea, and 2 successive intratracheal instillations, using 25-gauge needles, are performed.

Rats are allocated randomly to receive 0.1 mL of either saline or solution of the inhibitor peptide variant or composition of this disclosure at various doses and, 5 min later, a 0.5 mL/kg concentration of either saline or bacterial solution (P. aeruginosa). Thus, rats with pneumonia (the control group), rats with pneumonia treated with the of the inhibitor peptide variant or composition of the invention (the Test groups), and normal rats (the sham group) are studied.

The mechanical ventilation is started 18 h after the intratracheal instillations. Gibot et al. (2006). The rats are anesthetized and in addition to the biological and histological analyses, a different set of rats is used to assess survival. BAL, differential cell count, and histological examination. BAL is performed and the total number of lung cells is counted using a standard hemocytometer, and cytospin preparations are made. The cells are air-dried and stained with May-Grunwald Giemsa. Differential cell counts on 200 cells are made using standard morphological criteria. Histopathological examinations are performed. Arterial blood gases and lactate concentrations are determined hourly 30 min after the initiation of mechanical ventilation. Concentrations of TNF-a, IL-1b, and IL-6 in the plasma and the BAL fluid are analyzed as are plasma and BAL fluid D-dimer and thrombin-antithrombin complex (TATc) concentrations. All rats are administered a volume of saline corresponding to the volume of blood drawn after sampling.

Comparisons between 2 groups are made using Student's t test. Comparisons between all groups are made using a 1-way analysis of variance. Survival curves are compared using the log-rank test. A 2-tailed value of P<0.05 is considered significant The data are expressed as mean+/−SD. All analyses are performed with GraphPad Prism software.

In certain embodiments, cell surface receptor inhibitor peptide variants and compositions of this disclosure tested in this animal model are anticipated to be beneficial during P. aeruginosa pneumonia in rats in attenuating lung and systemic inflammatory responses. In one embodiment, said cell surface receptor inhibitor peptide variants and compositions are TREM-2 inhibitor peptide variants and compositions. In one embodiment, said cell surface receptor inhibitor peptide variants and compositions are combinatorial TREM-1 and TREM-2 concurrent inhibitor peptide variants and compositions. In one embodiment, better effect is anticipated for targeted LPC-bound trifunctional peptide variants and compositions of the invention compared with free peptide variants. In one embodiment, said TREM-2 inhibitor peptide is free peptide IA9. In one embodiment, said targeted LPC-bound trifunctional TREM-2 inhibitor peptide variant is IA31-LPC. In one embodiment, said combinatorial TREM-1 and TREM-2 concurrent inhibitor peptide is GA18.

### Example 17

### Efficacy in Protection Against Radiation-Induced Mortality

Studies of the peptide variants and compositions of this disclosure to demonstrate that said peptide variants and compositions can prevent and treat acute or late radiation effects including acute and chronic lung injuries including but not limited to ARDS and significantly extend survival of irradiated animals are performed using the standard methods. See, e.g. (Jackson et al. 2012, Kumar et al. 2018, Jin et al. 2020).

To test the peptide variants and compositions of this disclosure as a prophylactic and/or therapeutic ARS medical countermeasure (MCM), the studies are performed to determine the recommended therapeutic dose and schedule for the test article to improve 30-day survival in total body irradiated (TBI) mice (e.g., C57BL/6J mice) exposed to LD70/30 TBI dose when administered prior or after exposure.

Briefly, a single, randomized, blinded, vehicle-controlled study is conducted to evaluate the efficacy of each TA for the treatment of hematopoietic acute radiation syndrome (H-ARS) following TBI using a C57BL/6J mouse model. C57BL/6J at 10-12 weeks of age at the time of irradiation are randomized and be exposed to a single TBI dose of 8.0 Gy of 320 kV x-rays. The dose is sufficient to induce an estimated 70% mortality by day 30, relative to the day of irradiation (day 0) in the vehicle-treated arm. In the TA groups, mice are i.p. or subcutaneously treated with the peptide variants and compositions of the invention at various doses and timepoints before (preventative model) or after TBI (therapeutic model). In certain embodiments, said peptide variants and compositions of the invention are GF9, IA9, GA18, GA31-LPC or IA31-LPC.

The duration of the in-life phase is 45 days to allow progression of ARS towards full hematopoietic recovery. The primary endpoint is 30 day survival. Secondary endpoints include body weight loss and serum cytokine (TNFa, IL-6, IL-1b, and CSF-1) values in TA-treated and vehicle-treated mice. Ten animals per group will have serum samples collected on Day 1 before dosing, and on Day 2, 24 hr after dosing. All animals will have serum samples collected at sacrifice. Samples will be stored at −80° C. prior cytokine analysis. Formalin-fixed, paraffin-embedded (FFPE) tissue specimens (lung, liver, colon, small intestine, kidney, heart) from 45-day survivors are prepared, sectioned, stained for H&E, F4/80 and TREM-1 and/or TREM-2 and analyzed.

In certain embodiments, cell surface receptor inhibitor peptide variants and compositions of this disclosure tested in this animal model are anticipated to significantly extend survival in TA-treated mice exposed to LD70/30 TBI dose as compared to those treated with vehicle (PBS, pH 7.4). In one embodiment, better effect is anticipated for targeted LPC-bound trifunctional peptide variants and compositions of the invention compared with free peptide variants. In one embodiment, said cell surface receptor inhibitor peptide variants and compositions are TREM-2 inhibitor peptide variants and compositions. In one embodiment, said TREM-2 inhibitor peptide is free peptide IA9. In one embodiment, said targeted LPC-bound trifunctional TREM-2 inhibitor peptide variant is IA31-LPC. In one embodiment, said cell surface receptor inhibitor peptide variants and compositions are combinatorial TREM-1 and TREM-2 concurrent inhibitor peptide variants and compositions. In one embodiment, said combinatorial TREM-1 and TREM-2 concurrent inhibitor peptide is GA18.

Radiation pneumonitis is expected to be the predominant and lethal response between 3 and 6 months after whole thorax irradiation (Jackson et al. 2012). The clinically relevant measurable parameter for radiation pneumonitis in a mouse model is mortality.

Briefly, female C57L/J, CBA/J, and C57BL/6J mice at 8-10 wk of age (about 20 g) are randomized, anesthetized for irradiation and irradiated. Radiation dose and uniformity of distribution are determined prior to initiation of the study. Radiation is delivered to the thorax through adjustable apertures with 8-mm lead shielding of the head, abdomen, and forelimbs. Sham-irradiated animals are treated in the same way, except that the radiation source is not turned on.

In the TA groups, mice are i.p. or subcutaneously treated with the peptide variants and compositions of the invention at various doses and timepoints before (preventative model) or after TBI (therapeutic model). In certain embodiments, said peptide variants and compositions of the invention are GF9, IA9, GA18, GA31-LPC or IA31-LPC. Pulmonary function was assessed prior to radiation exposure and every two weeks thereafter during the in vivo portion of the study. Subsets of mice in each group are euthanized at a predetermined time point of 10 or 14 wk (n=5/group). The primary endpoint is day survival. Secondary endpoints include body weight loss and serum cytokine (TNFa, IL-6, IL-1b, and CSF-1) values in TA-treated and vehicle-treated mice. Ten animals per group will have serum samples collected before and after dosing. All animals will have serum samples collected at sacrifice. At the time of euthanasia, a bilateral thoracotomy is performed.

The left lobe is inflated through the bronchus with 10% neutral buffered formalin, paraffin embedded, sectioned in 5-um sections. The right lobe and heart are snap-frozen in liquid nitrogen. H&E-stained sections are evaluated for inflammatory cells, alveolar capillary distension or congestion, the presence of hyaline membranes, and alveolar wall thickness. Scoring of perivascular and alveolar inflammation is performed. Perivascular inflammation is scored on a scale of 0-4, where a score of 0 indicates no cell layers, while a score of 4 denotes four or more layers of inflammatory cells around the vessel. Alveolar inflammation is scored on a scale of 0-5 with 0 being no alveolar inflammation and 5 being complete consolidation of the tissue. Semi-quantitative assessment of the degree of interstitial fibrosis is determined using a predetermined numerical scale of 0-8 based on the Ashcroft scoring method.

In certain embodiments, cell surface receptor inhibitor peptide variants and compositions of this disclosure tested in this animal model are anticipated to significantly extend survival in TA-treated irradiated mice as compared to those treated with vehicle (PBS, pH 7.4). In one embodiment, better effect is anticipated for targeted LPC-bound trifunctional peptide variants and compositions of the invention compared with free peptide variants. In one embodiment, said cell surface receptor inhibitor peptide variants and compositions are TREM-2 inhibitor peptide variants and compositions. In one embodiment, said TREM-2 inhibitor peptide is free peptide IA9. In one embodiment, said targeted LPC-bound trifunctional TREM-2 inhibitor peptide variant is IA31-LPC. In one embodiment, said cell surface receptor inhibitor peptide variants and compositions are combinatorial TREM-1 and TREM-2 concurrent inhibitor peptide variants and compositions. In one embodiment, said combinatorial TREM-1 and TREM-2 concurrent inhibitor peptide is GA18.

### Example 18

### Efficacy Testing in Mouse Models of Sarcoma and Colorectal Cancer

TREM-2 is expressed in tumor macrophages in over 200 human cancer cases and inversely correlates with prolonged survival for sarcoma and colorectal cancer (Molgora et al. 2020). Studies of the peptide variants and compositions of this disclosure in mouse models of sarcoma and colorectal cancer are performed using the standard methods well known in the art (Molgora et al. 2020) to demonstrate that said peptide variants and compositions disclosed herein are effective in inhibiting TREM-2-mediated inflammatory response by ablation of macrophage inflammation and/or attenuation of cancer using established methods.

Briefly, male wild-type C57BL/6J mice are injected at 8 weeks of age with MCA/1956 (3-methylcholanthrene-induced sarcoma) and MC38 (colorectal cancer) cell lines. MCA/1956 or MC38 cells in PBS are injected subcutaneously (10⁶ cells/mouse in 100 ul PBS) in the flank. Mice are monitored every day and tumors are measured by caliper every other day. Mice are sacrificed at day 10, at day 24 or when tumors reached 1.5 cm of diameter.

Mice are i.p. treated with anti-PD1 antibody (200 ug/mouse) every 3 days, starting at day 3 or day 8 after tumor injection. Mice are treated i.p. with peptide variants and compositions disclosed herein every 2 days, starting at day 2 after tumor injection.

In certain embodiments, cell surface receptor inhibitor peptide variants and compositions of this disclosure tested in this animal model are anticipated to curb tumor growth and foster regression when combined with anti-PD-1. In one embodiment, said cell surface receptor inhibitor peptide variants and compositions are TREM-2 inhibitor peptide variants and compositions. In one embodiment, said cell surface receptor inhibitor peptide variants and compositions are combinatorial TREM-1 and TREM-2 concurrent inhibitor peptide variants and compositions.

In one embodiment, better effect is anticipated for targeted LPC-bound trifunctional peptide variants and compositions of the invention compared with free peptide variants. In one embodiment, said TREM-2 inhibitor peptide is free peptide IA9. In one embodiment, said targeted LPC-bound trifunctional TREM-2 inhibitor peptide variant is IA31-LPC. In one embodiment, said combinatorial TREM-1 and TREM-2 concurrent inhibitor peptide is GA18.

### EQUIVALENTS

Those skilled in the art will recognize, or be able to assume using no more than routine experimentation, many equivalents to the specific embodiments of the invention described herein. Such equivalents are intended to be encompassed by the following claims.

### INCORPORATION BY REFERENCE

All of the patents and publications cited herein are hereby incorporated by reference. Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself, and, each of these documents or references (“herein-cited references”), as well as each document or reference cited in each of the herein-cited references (including any manufacturer's specifications, instructions, etc.), is hereby expressly incorporated herein by reference.

### U.S. PATENT DOCUMENTS

Broersma, et al., U.S. Pat. No. 5,681,925, Oct. 28, 1997
Castaigne, et al., U.S. Pat. No. 9,161,988, Oct. 20, 2015
Castaigne, et al., U.S. Pat. No. 8,921,314, Dec. 30, 2014
Castaigne, et al., U.S. Pat. No. 9,173,891, Nov. 3, 2015
Caulfield, U.S. Pat. No. 8,114,613, Feb. 14, 2012
Eggink and Hoober, U.S. Pat. No. 7,811,995, Oct. 12, 2010 40
Eggink and Hoober, U.S. Pat. No. 8,496,942, Jul. 30, 2013
Elmore, U.S. Pat. No. 4,749,742, Jun. 7, 1988
Faure, et al., U.S. Pat. No. 8,013,116, Sep. 6, 2011
Faure, et al., U.S. Pat. No. 9,273,111, Mar. 1, 2016
Faure, et al., U.S. Pat. No. 10,603,357, Mar. 31, 2020 45
Gibot, et al., U.S. Pat. No. 9,657,081, May 23, 2017
Gibot and Derive, U.S. Pat. No. 9,815,883, Nov. 14, 2017
Gibot and Derive, U.S. Pat. No. 9,255,136, Feb. 9, 2016
Hazen, et al., U.S. Pat. No. 8,338,110, Dec. 25, 2012
Kas, et al. U.S. Pat. No. 8,697,370, April 15,2014 50
Kuai, et al. US2008/0247955. 2008.
Low and Turk, U.S. Pat. No. 8,916,167, Dec. 23, 2014
Manolios, U.S. Pat. No. 6,057,294, May 2, 2000
Manolios, U.S. Pat. No. 7,192,928, Mar. 20, 2007
Manolios, et al. US 20120077732. 2012 Manolios. US 20100267651. 2010
Morgan and Pandha. US 2012/5 0177672 A1. 2012
Schiffman and Altman, U.S. Pat. No. 4,427,660, Jan. 24, 1984
Sigalov, U.S. Pat. No. 8,278,271, Oct. 2, 2012
Sigalov, U.S. Pat. No. 8,614,188, Dec. 24, 2013
Sigalov. US 20130039948. 2013.
Sigalov, U.S. Pat. No. 9,981,004, May 29, 2018
Sigalov, U.S. Pat. No. 8,513,185, Aug. 20, 2013
Sigalov. US 20110256224. 2011.
Sigalov. US 20130045161. 2013.
Stennicke, et al. U.S. Pat. No. 10,189,904, January 29,2019
Urso, et al., U.S. Pat. No. 9,127,064, Sep. 8, 2015
Wu and Chi, U.S. Pat. No. 9,387,257, Jul. 12, 2016
Wu, et al., U.S. Pat. No. 8,415,453, Apr. 9, 2013 20

### FOREIGN PATENT DOCUMENTS

James and Andreasson, WO 2017083682A1, Publication Date: May 18, 2017,

### PUBLICATIONS

Ali et al. Bioconjug Chem 2005, 16:1556-63.
Amon et al. Biochim Biophys Acta 2006, 1763:879-88.
Annane et al. Am J Respir Crit Care Med 2013, 187:1091-7.
Arroyo-Hernandez et al. BMC Pulm Med 2021, 21:9.
Atzeni et al. Autoimmun Rev 2012, 12:225-9.
Binnewies et al. Cell Rep 2021, 37:109844.
Bouchon et al. Nature 2001, 410:1103-7.
Brand et al. Nat Protoc 2007, 2:1269-75.
Braun et al. Curr Opin Rheumatol 2009, 21:216-23.
Bresnihan et al. J Rheumatol 2007, 34:620-2.
Brynjolfsson et al. Inflamm Bowel Dis 2016, 22:1803-11.
Cavaillon et al. Scand J Infect Dis 2003, 35:535-44.
Channappanavar et al. Semin Immunopathol 2017, 39:529-39.
Chen et al. Am J Respir Crit Care Med 2013, 188:201-12.
Cheng et al. Front Immunol 2021, 12:646523.
Choy et al. Nat Rev Rheumatol 2013, 9:154-63.
Collier et al. Scand J Immunol 2006, 64:388-91.
Colonna Nat Rev Immunol 2003, 3:445-53.
Cope et al. Clin Exp Rheumatol 2007, 25:S4-11.
Cuvier et al. Br J Clin Pharmacol 2018, 84:2270-79.
de Oliveira et al. Inflammopharmacology 2022.
Deczkowska et al. Cell 2020, 181:1207-17.
Denning et al. Mol Med 2020a, 26:121.
Denning et al. JCI Insight 2020b, 5.
Denning et al. Surgery 2020c, 168:478-85.
Derive et al. Shock 2013, 39:176-82.
Derive et al. Anesthesiology 2014, 120:935-42.
Dower et al. J Immunol 2008, 180:3520-34.
Edrees et al. Clin Exp Rheumatol 2005, 23:469-74.
Elgert et al. J Leukoc Biol 1998,64:275-90.
Ferat-Osorio et al. J Surg Res 2008, 150:110-7.
Ford et al. Front Oncol 2021, 11:662723.
Ford et al. Curr Opin Immunol 2009, 21:38-46.
Francois et al. Am J Med 1997, 103:114-20.
Francois et al. Intensive Care Med 2020, 46:1425-37.
Franz et al. Ann Rheum Dis 2005, 64:798-800.
Frohlich Biomedicines 2021, 9.
Gaffo et al. Am J Health Syst Pharm 2006, 63:2451-65.
Gallop et al. Infect Immun 2021, 89:e0012621.
Galloway et al. Ann Rheum Dis 2011, 70:1810-4.
Garrood et al. Arthritis Rheum 2006, 54:1055-60.
Gawish et al. Faseb J 2015, 29:1247-57.
Gibot Crit Care 2005, 9:485-9.
Gibot Semin Respir Crit Care Med 2006a, 27:29-33.
Gibot Curr Opin Investig Drugs 2006b, 7:438-42.
Gibot et al. J Infect Dis 2006a, 194:975-83.
Gibot et al. Infect Immun 2006b, 74:2823-30.
Gibot et al. Shock 2009, 32:633-7.
Gibot et al. Eur J Immunol 2007, 37:456-66.
Gogos et al. J Infect Dis 2000, 181:176-80.
Gong et al. medRxiv 2020:2020.02.25.20025643.
Gratuze et al. Mol Neurodegener 2018, 13:66.
Guo et al. Mil Med Res 2020, 7:11.
Hommes et al. J Pathol 2014, 233:357-67.
Huang et al. J Transl Med 2021, 19:496.
Jackson et al. BJR Suppl 2005, 27:161-8.
Jackson et al. Health Phys 2012, 103:463-73.
Jin et al. Cancers (Basel) 2020, 12.
Joffre et al. J Am Coll Cardiol 2016, 68:2776-93.
Johnson et al. Neuro Oncol 2017, 19:vi249.
Kinne et al. Arthritis Res 2000, 2:189-202.
Kinne et al. Arthritis Res Ther 2007, 9:224.
Klesney-Tait et al. Nat Immunol 2006, 7:1266-73.
Kumar et al. Health Phys 2018, 115:65-76.
Lagler et al. J Immunol 2009, 183:2027-36.
Lawson et al. Am J Pathol 2005, 167:1267-77.
Lazzari et al. J Cancer Prev Curr Res 2020, 11:121-4.
Liu et al. Nat Immunol 2019, 20:1023-34.
Liu et al. J Biol Chem 2002, 277:21576-84.
Liu et al. Front Physiol 2017, 8:193.
Macia et al. Rep Pract Oncol Radiother 2011, 16:123-30.
Malaviya et al. Ann N Y Acad Sci 2016, 1374:168-75.
Mark et al. International Journal of Radiation Oncology⋅Biology⋅Physics 2019, 105:E646-E47.
Marshall Trends Mol Med 2014, 20:195-203.
Matthay et al. Lancet Respir Med 2020.
Matthay et al. Nat Rev Dis Primers 2019, 5:18.
Mehta et al. Chest 2013, 144:643A.
Meziani et al. Oncoimmunology 2018a, 7:e1494488.
Meziani et al. Eur Respir J 2018b, 51.
Molgora et al. Cell 2020, 182:886-900 e17.
Ngkelo et al. J Inflamm (Lond) 2012, 9:1.
O'Dell N Engl J Med 2004, 350:2591-602.
Oberholzer et al. Shock 2005, 23:488-93.
Opal et al. Am J Respir Crit Care Med 2013, 187:1041-3.
Palazzo et al. Dimens Crit Care Nurs 2012, 31:1-6.
Peck et al. Inflamm Res 2009, 58:89-93.
Pelham et al. Expert Opin Ther Pat 2014, 24:1383-95.
Perry et al. Inhal Toxicol 2021, 33:221-33.
Poston et al. Jama 2020.
Qian et al. Int J Clin Exp Med 2014, 7:1650-8.
Qiu et al. Front Immunol 2021, 12:716710.
Remick Curr Pharm Des 2003, 9:75-82.
Reynolds Am J Respir Crit Care Med 2005, 171:98-102.
Riedemann et al. J Clin Invest 2003, 112:460-7.
Rojas et al. Biochim Biophys Acta 2018, 1864:2761-68.
Sadeghi et al. Toxicol Lett 2020, 333:115-29.
Sadikot et al. Biomed Hub 2017, 2:1-12.
Schenk et al. J Clin Invest 2007, 117:3097-106.
Schmid-Schonbein Annu Rev Biomed Eng 2006, 8:93-131.
Shen et al. Sci Rep 2016, 6:28672.
Shen et al. Mol Pharm 2017a, 14:4572-82.
Shen et al. J Cell Mol Med 2017b, 21:2524-34.
Shen et al. Biomed Res Int 2021, 2021:6635452.
Sigalov Trends Immunol 2004, 25:583-9.
Sigalov Trends Pharmacol Sci 2006, 27:518-24.
Sigalov Self Nonself 2010a, 1:4-39.
Sigalov Self Nonself 2010b, 1:192-224.
Sigalov Contrast Media Mol Imaging 2014a, 9:372-82.
Sigalov Int Immunopharmacol 2014b, 21:208-19.
Sigalov Drug Discovery Today 2020, 25:1298-306.
Siskind et al. Surgery 2022.
Smolen et al. Ann Rheum Dis 2010, 69:964-75.
Standiford et al. Transl Res 2016, 167:183-91.
Starr et al. PLoS One 2014, 9:e115705.
Suarez-Almazor et al. Cochrane Database Syst Rev 2000:CD000957.
Sun et al. Neuroscience 2019, 406:369-75.
Tak et al. Arthritis Rheum 1997, 40:217-25.
Takeuchi et al. Ann Rheum Dis 2011, 70:1208-15.
Tammaro et al. Pharmacol Ther 2017, 177:81-95.
Thomas-Ruddel et al. Anaesthesist 2020.
Tisoncik et al. Microbiol Mol Biol Rev 2012, 76:16-32.
Tornai et al. Hepatol Commun 2019, 3:99-115.
van Bremen et al. Diagn Pathol 2013, 8:24.
Varney et al. Melanoma Res 2005, 15:417-25.
Vlieghe et al. Drug Discov Today 2010, 15:40-56.
Wang et al. Inflamm Res 2021a, 70:971-80.
Wang et al. Ann Clin Lab Sci 2019, 49:249-56.
Wang et al. Cell Immunol 2012, 272:251-8.
Wang et al. World J Gastroenterol 2004, 10:2744-6.
Wang et al. Mediators Inflamm 2021b, 2021:2327721.
Weehuizen et al. PLoS Negl Trop Dis 2016, 10:e0004747.
Weinberger et al. Toxicol Appl Pharmacol 2016, 305:1-11.
Williams et al. Int J Radiat Biol 2011, 87:851-68.
Wluka et al. J Rheumatol 2000, 27:1864-71.
Wu et al. J Exp Med 2015, 212:681-97.
Wujtewicz et al. Anaesthesiol Intensive Ther 2020, 52:34-41.
Yuan et al. Am J Physiol Lung Cell Mol Physiol 2016, 310:L426-38.
Zeisberg et al. American journal of physiology. Cell physiology 2013, 304:C216-C25.
Zhou et al. Int Immunopharmacol 2013, 17:155-61.
Zhu et al. Microb Biotechnol 2019, 12:1337-45.

1. A peptide comprising an amino acid sequence having the general formula of R1-AA1-AA2-A1-A2-B-C-D1-D2-E-EE1-EE2-R2, wherein:
R1 is absent or is selected from the group consisting of N-terminal sugar conjugate and N-terminal lipid conjugate;
AA1 is absent or is selected from the group consisting of Arg, Arg-Arg, Arg-Arg-Arg and Arg-Arg-Arg-Arg;
AA2 is absent or is selected from the group consisting of Lys, Lys-Lys, Lys-Lys-Lys and Lys-Lys-Lys-Lys;
A1 is an amino acid selected from the group consisting of Pro, Cys, Leu, Ala, Val, Ile, Met, Trp, Gly and Phe, a two amino acid peptide, a three amino acid peptide, a four amino acid peptide, a five amino acid peptide, a six amino acid peptide and a seven amino acid peptide, said peptide consisting of Pro, Cys, Leu, Ala, Val, Ile, Met, Trp, Gly and Phe in any combination;
A2 is absent or is a positively charged amino acid selected from the group comprising Arg, Lys and His;
B is selected from the group consisting of Pro, Cys, Leu, Ala, Val, Ile, Met, Trp, Gly and Phe, a two amino acid peptide and a three amino acid peptide, said peptide consisting of Pro, Cys, Leu, Ala, Val, Ile, Met, Trp, Gly and Phe in any combination;
C is a positively charged amino acid selected from the group comprising Arg, Lys and His;
D1 is selected from the group consisting of Pro, Cys, Leu, Ala, Val, Ile, Met, Trp, Gly and Phe, a two amino acid peptide and a three amino acid peptide, said peptide consisting of Pro, Cys, Leu, Ala, Val, Ile, Met, Trp, Gly and Phe in any combination;
D2 is absent or is a positively charged amino acid selected from the group comprising Arg, Lys and His;
E is an amino acid selected from the group consisting of Pro, Cys, Leu, Ala, Val, Ile, Met, Trp, Gly and Phe, a two amino acid peptide, a three amino acid peptide, a four amino acid peptide, a five amino acid peptide, a six amino acid peptide and a seven amino acid peptide, said peptide consisting of Pro, Cys, Leu, Ala, Val, Ile, Met, Trp, Gly and Phe in any combination;
EE1 is absent or is selected from the group consisting of Arg, Arg-Arg, Arg-Arg-Arg and Arg-Arg-Arg-Arg;
EE2 is absent or is selected from the group consisting of Lys, Lys-Lys, Lys-Lys-Lys and Lys-Lys-Lys-Lys; and
R2 is absent or is C-terminal lipid conjugate.

2. The peptide of claim 1, wherein the distance between A2 and C1 is one to three amino acid residues.

3. The peptide of claim 1, wherein the distance between C1 and D2 is one to three amino acid residues.

4. (canceled)

5. The peptide of claim 1, wherein said N-terminal lipid conjugate is selected from the group consisting of 1-amino-glucose succinate, 2-aminododecanoate and myristoylate conjugates.

6. The peptide of claim 1, wherein said C-terminal lipid conjugate is selected from the group consisting of Gly-Tris-monopalmitate, Gly-Tris-dipalmitate and Gly-Tris-tripalmitate conjugates.

7. The peptide of claim 1, wherein said peptide is attached to a carrier molecule.

8. The peptide of claim 1, wherein said peptide is conjugated at a free amine group with a polyalkylene glycol.

9. The peptide of claim 8, wherein said polyalkylene glycol is polyethylene glycol.

10. The peptide of claim 1, wherein one or more amino acids is a D-amino acid.

11. The peptide of claim 1, wherein said peptide is a cyclic peptide or a dimer peptide.

12-13. (canceled)

14. A method, comprising:
a) providing;
i) a patient exhibiting at least one symptom of a lung disease; and
ii) a pharmaceutically acceptable composition comprising a TREM-2 inhibitor; and
b) administering said composition to said patient such that said at least one symptom is reduced.

15. The method of claim 14, wherein said TREM-2 inhibitor comprises a peptide with an amino acid sequence having the general formula of R1-AA1-AA2-A1-A2-B-C-D1-D2-E-EE1-EE2-R2.

16. The method of claim 14, wherein said pharmaceutically acceptable composition further comprises a lipopeptide/lipoprotein complex.

17. The method of claim 16, wherein said lipopeptide/lipoprotein complex further comprises a complex of a peptide selected from the group consisting of IFLIKILAAPLGEEMRDRARAHVDALRTHLA and IFLIKILAAPYLDDFQKKWQEEMELYRQKVE.

18. The method of claim 17, wherein the methionine residues of said IFLIKILAAPLGEEMRDRARAHVDALRTHLA and said IFLIKILAAPYLDDFQKKWQEEMELYRQKVE are sulfoxidized.

19. (canceled)

20. The method of claim 17 where said lipopeptide/lipoprotein complex comprises a lipid selected from the group consisting of phospholipid, cholesterol, cholesteryl oleate, 1-palmitoyl-2-oleoyl-sn-glycero-3-phosphocholine, 1,2-dipalmitoyl-sn-glycero-3-phosphocholine, egg yolk L-a-phosphatidylcholine, soy L-α-phosphatidylcholine and any combination thereof.

21. The method of claim 14, wherein said at least one symptom is selected from the group consisting of shortness of breath, inflammation of lung parenchyma, infiltration of neutrophils into pulmonary airspaces, oxidative stress, disruption of the endothelial barriers, disruption of the epithelial barriers, pulmonary epithelial lining damage, lung fibrosis, progressive hypoxemia and dyspnea.

22. The method of claim 14, where said lung disease is an acute respiratory distress syndrome.

23. The method of claim 22, wherein said acute respiratory distress syndrome is induced by a medical condition selected from the group consisting of sepsis, bacterial pneumonia, viral pneumonia, inhalation of harmful substances, major injury, burns, blood transfusions, near drowning, aspiration of gastric contents, pancreatitis, intravenous drug use, abdominal trauma and chronic alcoholism.

24. The method of claim 14, where said lung disease is a chemical injury.

25. The method of claim 24, wherein said chemical injury is selected from the group comprising a mustard gas injury, a phosgene injury and a chlorine injury.

26. The method of claim 14, wherein said lung disease is a radiation lung injury or an ionizing radiation injury.

27-36. (canceled)
